SEQUENCE LISTING (463 sequences)

SEQ ID NO: 1 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence [sequence omitted]

SEQ ID NO: 3 - protein, 30 aa, Artificial Sequence
  source: /note=“Description of Artificial Sequence Synthetic polypeptide”
QVQLVQSGAEVKKPGASVKVSCKASGYIFS

SEQ ID NO: 4 - protein, 30 aa, Artificial Sequence
  source: /note=“Description of Artificial Sequence Synthetic polypeptide”
QVQLVQSGAEVKKPGASVKVSCKASGHIFS

SEQ ID NO: 5 - protein, 30 aa, Artificial Sequence
  source: /note=“Description of Artificial Sequence Synthetic polypeptide”
QVQLVQSGAEVKKPGASVKVSCKASGYTFS

SEQ ID NO: 6 - protein, 30 aa, Artificial Sequence
  source: /note=“Description of Artificial Sequence Synthetic polypeptide”
QVQLVQSGAEVKKPGASVKVSCKASGHTFS

SEQ ID NO: 7 - protein, 5 aa, Artificial Sequence
  source: /note=“Description of Artificial Sequence Synthetic peptide”
NYWIQ

SEQ ID NO: 8 - protein, 5 aa, Artificial Sequence
  source: /note=“Description of Artificial Sequence Synthetic peptide”
HYWIQ

SEQ ID NO: 9 - protein, 5 aa, Artificial Sequence
  source: /note=“Description of Artificial Sequence Synthetic peptide”
NYWHQ

SEQ ID NO: 10 - protein, 5 aa, Artificial Sequence
  source: /note=“Description of Artificial Sequence Synthetic peptide”
HYWHQ

SEQ ID NO: 11 - protein, 14 aa, Artificial Sequence
  source: /note=“Description of Artificial Sequence Synthetic peptide”
WVRQAPGQGLEWMG

SEQ ID NO: 12 - protein, 17 aa, Artificial Sequence
  source: /note=“Description of Artificial Sequence Synthetic peptide”
EILPGSGSTEYTENFKD

SEQ ID NO: 13 - protein, 17 aa, Artificial Sequence
  source: /note=“Description of Artificial Sequence Synthetic peptide”
EIHPGSGSTEYTENFKD

SEQ ID NO: 14 - protein, 17 aa, Artificial Sequence
  source: /note=“Description of Artificial Sequence Synthetic peptide”
EILPGSGHTEYTENFKD

SEQ ID NO: 15 - protein, 17 aa, Artificial Sequence
  source: /note=“Description of Artificial Sequence Synthetic peptide”
EIHPGSGHTEYTENFKD

SEQ ID NO: 16 - protein, 32 aa, Artificial Sequence
  source: /note=“Description of Artificial Sequence Synthetic polypeptide”
RVTMTRDTSTSTVYMELSSLRSEDTAVYYCAR

SEQ ID NO: 17 - protein, 32 aa, Artificial Sequence
  source: /note=“Description of Artificial Sequence Synthetic polypeptide”
RVTMTRDTSISTAYMELSSLRSEDTAVYYCAR

SEQ ID NO: 18 - protein, 13 aa, Artificial Sequence
  source: /note=“Description of Artificial Sequence Synthetic peptide”
YFFGSSPNWYFDV

SEQ ID NO: 19 - protein, 13 aa, Artificial Sequence
  source: /note=“Description of Artificial Sequence Synthetic peptide”
YHFGSSPNWYFDV

SEQ ID NO: 20 - protein, 11 aa, Artificial Sequence
  source: /note=“Description of Artificial Sequence Synthetic peptide”
WGQGTLVTVSS

SEQ ID NO: 21 - protein, 122 aa, Artificial Sequence [sequence omitted]

SEQ ID NO: 22 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 23 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 24 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 25 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 26 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 27 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 28 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 29 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 30 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 31 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 32 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 33 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 34 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 35 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 36 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 37 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 38 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 39 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 40 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 41 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 42 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 43 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 44 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 45 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 46 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 47 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 48 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 49 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 50 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 51 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 52 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGSTEYTENFKDRVTMTRDTSTSTVYMELSSLRSEDTAVYYCARYFFGSSPNWYFDVWGQGTLVTVSS

SEQ ID NO: 53 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 54 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 55 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 56 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 57 - protein, 122 aa, Artificial Sequence [sequence omitted]

SEQ ID NO: 58 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 59 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 60 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 61 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 62 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 63 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 64 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 65 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 66 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 67 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 68 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 69 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 70 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 71 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 72 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 73 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 74 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 75 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 76 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 77 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 78 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 79 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 80 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 81 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 82 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 83 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 84 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGHTEYTENFKDRVTMTRDTSTSTVYMELSSLRSEDTAVYYCARYFFGSSPNWYFDVWGQGTLVTVSS

SEQ ID NO: 85 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 86 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 87 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 88 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 89 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 90 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 91 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 92 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 93 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 94 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 95 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 96 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 97 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 98 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 99 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 100 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGSTEYTENFKDRVTMTRDTSISTAYMELSSLRSEDTAVYYCARYFFGSSPNWYFDVWGQGTLVTVSS

SEQ ID NO: 101 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 102 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 103 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 104 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 105 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 106 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 107 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 108 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 109 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 110 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 111 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 112 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 113 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 114 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 115 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 116 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGSTEYTENFKDRVTMTRDTSISTAYMELSSLRSEDTAVYYCARYFFGSSPNWYFDVWGQGTLVTVSS

SEQ ID NO: 117 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 118 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 119 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 120 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 121 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 122 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 123 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 124 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 125 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 126 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 127 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 128 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 129 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 130 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 131 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 132 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGHTEYTENFKDRVTMTRDTSISTAYMELSSLRSEDTAVYYCARYFFGSSPNWYFDVWGQGTLVTVSS

SEQ ID NO: 133 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 134 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 135 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 136 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 137 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 138 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 139 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 140 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 141 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 142 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 143 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 144 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 145 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 146 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 147 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 148 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGHTEYTENFKDRVTMTRDTSISTAYMELSSLRSEDTAVYYCARYFFGSSPNWYFDVWGQGTLVTVSS

SEQ ID NO: 149 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 150 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 151 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 152 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 153 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 154 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 155 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 156 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 157 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 158 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 159 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 160 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 161 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 162 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 163 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 164 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGSTEYTENFKDRVTMTRDTSTSTVYMELSSLRSEDTAVYYCARYHFGSSPNWYFDVWGQGTLVTVSS

SEQ ID NO: 165 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 166 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 167 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 168 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 169 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 170 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 171 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 172 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 173 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 174 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 175 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 176 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 177 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 178 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 179 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 180 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGSTEYTENFKDRVTMTRDTSTSTVYMELSSLRSEDTAVYYCARYHFGSSPNWYFDVWGQGTLVTVSS

SEQ ID NO: 181 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 182 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 183 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 184 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 185 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 186 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 187 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 188 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 189 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 190 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 191 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 192 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 193 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 194 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 195 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 196 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGHTEYTENFKDRVTMTRDTSTSTVYMELSSLRSEDTAVYYCARYHFGSSPNWYFDVWGQGTLVTVSS

SEQ ID NO: 197 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 198 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 199 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 200 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 201 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 202 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 203 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 204 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 205 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 206 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 207 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 208 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 209 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 210 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 211 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 212 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGHTEYTENFKDRVTMTRDTSTSTVYMELSSLRSEDTAVYYCARYHFGSSPNWYFDVWGQGTLVTVSS

SEQ ID NO: 213 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 214 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 215 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 216 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 217 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 218 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 219 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 220 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 221 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 222 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 223 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 224 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 225 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 226 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 227 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 228 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGSTEYTENFKDRVTMTRDTSISTAYMELSSLRSEDTAVYYCARYHFGSSPNWYFDVWGQGTLVTVSS

SEQ ID NO: 229 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 230 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 231 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 232 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 233 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 234 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 235 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 236 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 237 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 238 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 239 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 240 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 241 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 242 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 243 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 244 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGSTEYTENFKDRVTMTRDTSISTAYMELSSLRSEDTAVYYCARYHFGSSPNWYFDVWGQGTLVTVSS

SEQ ID NO: 245 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 246 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 247 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 248 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 249 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 250 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 251 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 252 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 253 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 254 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 255 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 256 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 257 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 258 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 259 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 260 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEILPGSGHTEYTENFKDRVTMTRDTSISTAYMELSSLRSEDTAVYYCARYHFGSSPNWYFDVWGQGTLVTVSS

SEQ ID NO: 261 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 262 - protein, 122 aa, Artificial Sequence
  source: /note=“Description of Artificial Sequence Synthetic polypeptide”
QVQLVQSGAEVKKPGASVKVSCKASGYIFSHYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 263 - protein, 122 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 264 - protein, 122 aa, Artificial Sequence
  source: /note=“Description of Artificial Sequence Synthetic polypeptide”
QVQLVQSGAEVKKPGASVKVSCKASGYIFSHYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 265 - protein, 122 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 266 - protein, 122 aa, Artificial Sequence
  source: /note=“Description of Artificial Sequence Synthetic polypeptide”
QVQLVQSGAEVKKPGASVKVSCKASGHIFSHYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 267 - protein, 122 aa, Artificial Sequence
  source: /note=“Description of Artificial Sequence Synthetic polypeptide”
QVQLVQSGAEVKKPGASVKVSCKASGHIFSNYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 268 - protein, 122 aa, Artificial Sequence
  source: /note=“Description of Artificial Sequence Synthetic polypeptide”
QVQLVQSGAEVKKPGASVKVSCKASGHIFSHYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 269 - protein, 122 aa, Artificial Sequence
  source: /note=“Description of Artificial Sequence Synthetic polypeptide”
QVQLVQSGAEVKKPGASVKVSCKASGYTFSNYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 270 - protein, 122 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 271 - protein, 122 aa, Artificial Sequence
  source: /note=“Description of Artificial Sequence Synthetic polypeptide”
QVQLVQSGAEVKKPGASVKVSCKASGYTFSNYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 272 - protein, 122 aa, Artificial Sequence
  source: /note=“Description of Artificial Sequence Synthetic polypeptide”
QVQLVQSGAEVKKPGASVKVSCKASGYTFSHYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 273 - protein, 122 aa, Artificial Sequence
  source: /note=“Description of Artificial Sequence Synthetic polypeptide”
QVQLVQSGAEVKKPGASVKVSCKASGHTFSNYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 274 - protein, 122 aa, Artificial Sequence
  source: /note=“Description of Artificial Sequence Synthetic polypeptide”
QVQLVQSGAEVKKPGASVKVSCKASGHTFSHYWIQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 275 - protein, 122 aa, Artificial Sequence
  source: /note=“Description of Artificial Sequence Synthetic polypeptide”
QVQLVQSGAEVKKPGASVKVSCKASGHTFSNYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 276 - protein, 122 aa, Artificial Sequence
  source: /note=“Description of Artificial Sequence Synthetic polypeptide”
QVQLVQSGAEVKKPGASVKVSCKASGHTFSHYWHQWVRQAPGQGLEWMGEIHPGSGHTEYTENFKDRVTMTRDTSISTAYMELSSLRSEDTAVYYCARYHFGSSPNWYFDVWGQGTLVTVSS

SEQ ID NO: 277 - protein, 23 aa, Artificial Sequence
  source: /note=“Description of Artificial Sequence Synthetic peptide”
DIQMTQSPSSLSASVGDRVTITC

SEQ ID NO: 278 - protein, 11 aa, Artificial Sequence
  source: /note=“Description of Artificial Sequence Synthetic peptide”
GASENIYGALN

SEQ ID NO: 279 - protein, 11 aa, Artificial Sequence
  source: /note=“Description of Artificial Sequence Synthetic peptide”
GAHENIYGALN

SEQ ID NO: 280 - protein, 11 aa, Artificial Sequence
  source: /note=“Description of Artificial Sequence Synthetic peptide”
GASENIYHALN

SEQ ID NO: 281 - protein, 11 aa, Artificial Sequence
  source: /note=“Description of Artificial Sequence Synthetic peptide”
GASENIYGAHN

SEQ ID NO: 282 - protein, 11 aa, Artificial Sequence
  source: /note=“Description of Artificial Sequence Synthetic peptide”
GAHENIYHALN

SEQ ID NO: 283 - protein, 11 aa, Artificial Sequence
  source: /note=“Description of Artificial Sequence Synthetic peptide”
GAHENIYGAHN

SEQ ID NO: 284 - protein, 11 aa, Artificial Sequence
  source: /note=“Description of Artificial Sequence Synthetic peptide”
GASENIYHAHN

SEQ ID NO: 285 - protein, 11 aa, Artificial Sequence
  source: /note=“Description of Artificial Sequence Synthetic peptide”
GAHENIYHAHN

SEQ ID NO: 286 - protein, 15 aa, Artificial Sequence
  source: /note=“Description of Artificial Sequence Synthetic peptide”
WYQQKPGKAPKLLIY

SEQ ID NO: 287 - protein, 7 aa, Artificial Sequence
  source: /note=“Description of Artificial Sequence Synthetic peptide”
GATNLAD

SEQ ID NO: 288 - protein, 7 aa, Artificial Sequence
  source: /note=“Description of Artificial Sequence Synthetic peptide”
GATNLAH

SEQ ID NO: 289 - protein, 32 aa, Artificial Sequence
  source: /note=“Description of Artificial Sequence Synthetic polypeptide”
GVPSRFSGSGSGTDFTLTISSLQPEDFATYYC

SEQ ID NO: 290 - protein, 9 aa, Artificial Sequence
  source: /note=“Description of Artificial Sequence Synthetic peptide”
QNVLNTPLT

SEQ ID NO: 291 - protein, 9 aa, Artificial Sequence
  source: /note=“Description of Artificial Sequence Synthetic peptide”
QNHLNTPLT

SEQ ID NO: 292 - protein, 9 aa, Artificial Sequence
  source: /note=“Description of Artificial Sequence Synthetic peptide”
QNVLHTPLT

SEQ ID NO: 293 - protein, 9 aa, Artificial Sequence
  source: /note=“Description of Artificial Sequence Synthetic peptide”
QNVLNHPLT

SEQ ID NO: 294 - protein, 9 aa, Artificial Sequence
  source: /note=“Description of Artificial Sequence Synthetic peptide”
QNHLHTPLT

SEQ ID NO: 295 - protein, 9 aa, Artificial Sequence
  source: /note=“Description of Artificial Sequence Synthetic peptide”
QNHLNHPLT

SEQ ID NO: 296 - protein, 9 aa, Artificial Sequence
  source: /note=“Description of Artificial Sequence Synthetic peptide”
QNVLHHPLT

SEQ ID NO: 297 - protein, 9 aa, Artificial Sequence
  source: /note=“Description of Artificial Sequence Synthetic peptide”
QNHLHHPLT

SEQ ID NO: 298 - protein, 11 aa, Artificial Sequence
  source: /note=“Description of Artificial Sequence Synthetic peptide”
FGQGTKVEIKR

SEQ ID NO: 299 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKR

SEQ ID NO: 300 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVLNTPLTFGQGTKVEIKR

SEQ ID NO: 301 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNVLNTPLTFGQGTKVEIKR

SEQ ID NO: 302 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNVLNTPLTFGQGTKVEIKR

SEQ ID NO: 303 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNVLNTPLTFGQGTKVEIKR

SEQ ID NO: 304 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVLNTPLTFGQGTKVEIKR

SEQ ID NO: 305 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNVLNTPLTFGQGTKVEIKR

SEQ ID NO: 306 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNVLNTPLTFGQGTKVEIKR

SEQ ID NO: 307 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNVLNTPLTFGQGTKVEIKR

SEQ ID NO: 308 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNVLNTPLTFGQGTKVEIKR

SEQ ID NO: 309 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNVLNTPLTFGQGTKVEIKR

SEQ ID NO: 310 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNVLNTPLTFGQGTKVEIKR

SEQ ID NO: 311 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNVLNTPLTFGQGTKVEIKR

SEQ ID NO: 312 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNVLNTPLTFGQGTKVEIKR

SEQ ID NO: 313 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNVLNTPLTFGQGTKVEIKR

SEQ ID NO: 314 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNVLNTPLTFGQGTKVEIKR

SEQ ID NO: 315 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNHLNTPLTFGQGTKVEIKR

SEQ ID NO: 316 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNHLNTPLTFGQGTKVEIKR

SEQ ID NO: 317 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNHLNTPLTFGQGTKVEIKR

SEQ ID NO: 318 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNHLNTPLTFGQGTKVEIKR

SEQ ID NO: 319 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNHLNTPLTFGQGTKVEIKR

SEQ ID NO: 320 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNHLNTPLTFGQGTKVEIKR

SEQ ID NO: 321 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNHLNTPLTFGQGTKVEIKR

SEQ ID NO: 322 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNHLNTPLTFGQGTKVEIKR

SEQ ID NO: 323 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNHLNTPLTFGQGTKVEIKR

SEQ ID NO: 324 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNHLNTPLTFGQGTKVEIKR

SEQ ID NO: 325 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNHLNTPLTFGQGTKVEIKR

SEQ ID NO: 326 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNHLNTPLTFGQGTKVEIKR

SEQ ID NO: 327 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNHLNTPLTFGQGTKVEIKR

SEQ ID NO: 328 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNHLNTPLTFGQGTKVEIKR

SEQ ID NO: 329 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNHLNTPLTFGQGTKVEIKR

SEQ ID NO: 330 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNHLNTPLTFGQGTKVEIKR

SEQ ID NO: 331 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLHTPLTFGQGTKVEIKR

SEQ ID NO: 332 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVLHTPLTFGQGTKVEIKR

SEQ ID NO: 333 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNVLHTPLTFGQGTKVEIKR

SEQ ID NO: 334 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNVLHTPLTFGQGTKVEIKR

SEQ ID NO: 335 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNVLHTPLTFGQGTKVEIKR

SEQ ID NO: 336 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVLHTPLTFGQGTKVEIKR

SEQ ID NO: 337 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNVLHTPLTFGQGTKVEIKR

SEQ ID NO: 338 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNVLHTPLTFGQGTKVEIKR

SEQ ID NO: 339 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNVLHTPLTFGQGTKVEIKR

SEQ ID NO: 340 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNVLHTPLTFGQGTKVEIKR

SEQ ID NO: 341 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNVLHTPLTFGQGTKVEIKR

SEQ ID NO: 342 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNVLHTPLTFGQGTKVEIKR

SEQ ID NO: 343 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNVLHTPLTFGQGTKVEIKR

SEQ ID NO: 344 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNVLHTPLTFGQGTKVEIKR

SEQ ID NO: 345 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNVLHTPLTFGQGTKVEIKR

SEQ ID NO: 346 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNVLHTPLTFGQGTKVEIKR

SEQ ID NO: 347 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLNHPLTFGQGTKVEIKR

SEQ ID NO: 348 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVLNHPLTFGQGTKVEIKR

SEQ ID NO: 349 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNVLNHPLTFGQGTKVEIKR

SEQ ID NO: 350 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNVLNHPLTFGQGTKVEIKR

SEQ ID NO: 351 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNVLNHPLTFGQGTKVEIKR

SEQ ID NO: 352 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVLNHPLTFGQGTKVEIKR

SEQ ID NO: 353 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNVLNHPLTFGQGTKVEIKR

SEQ ID NO: 354 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNVLNHPLTFGQGTKVEIKR

SEQ ID NO: 355 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNVLNHPLTFGQGTKVEIKR

SEQ ID NO: 356 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNVLNHPLTFGQGTKVEIKR

SEQ ID NO: 357 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNVLNHPLTFGQGTKVEIKR

SEQ ID NO: 358 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNVLNHPLTFGQGTKVEIKR

SEQ ID NO: 359 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNVLNHPLTFGQGTKVEIKR

SEQ ID NO: 360 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNVLNHPLTFGQGTKVEIKR

SEQ ID NO: 361 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNVLNHPLTFGQGTKVEIKR

SEQ ID NO: 362 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNVLNHPLTFGQGTKVEIKR

SEQ ID NO: 363 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNHLHTPLTFGQGTKVEIKR

SEQ ID NO: 364 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNHLHTPLTFGQGTKVEIKR

SEQ ID NO: 365 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNHLHTPLTFGQGTKVEIKR

SEQ ID NO: 366 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNHLHTPLTFGQGTKVEIKR

SEQ ID NO: 367 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNHLHTPLTFGQGTKVEIKR

SEQ ID NO: 368 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNHLHTPLTFGQGTKVEIKR

SEQ ID NO: 369 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNHLHTPLTFGQGTKVEIKR

SEQ ID NO: 370 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNHLHTPLTFGQGTKVEIKR

SEQ ID NO: 371 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNHLHTPLTFGQGTKVEIKR

SEQ ID NO: 372 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNHLHTPLTFGQGTKVEIKR

SEQ ID NO: 373 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNHLHTPLTFGQGTKVEIKR

SEQ ID NO: 374 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNHLHTPLTFGQGTKVEIKR

SEQ ID NO: 375 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNHLHTPLTFGQGTKVEIKR

SEQ ID NO: 376 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNHLHTPLTFGQGTKVEIKR

SEQ ID NO: 377 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNHLHTPLTFGQGTKVEIKR

SEQ ID NO: 378 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNHLHTPLTFGQGTKVEIKR

SEQ ID NO: 379 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNHLNHPLTFGQGTKVEIKR

SEQ ID NO: 380 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNHLNHPLTFGQGTKVEIKR

SEQ ID NO: 381 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNHLNHPLTFGQGTKVEIKR

SEQ ID NO: 382 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNHLNHPLTFGQGTKVEIKR

SEQ ID NO: 383 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNHLNHPLTFGQGTKVEIKR

SEQ ID NO: 384 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNHLNHPLTFGQGTKVEIKR

SEQ ID NO: 385 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNHLNHPLTFGQGTKVEIKR

SEQ ID NO: 386 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNHLNHPLTFGQGTKVEIKR

SEQ ID NO: 387 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNHLNHPLTFGQGTKVEIKR

SEQ ID NO: 388 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNHLNHPLTFGQGTKVEIKR

SEQ ID NO: 389 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNHLNHPLTFGQGTKVEIKR

SEQ ID NO: 390 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNHLNHPLTFGQGTKVEIKR

SEQ ID NO: 391 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNHLNHPLTFGQGTKVEIKR

SEQ ID NO: 392 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNHLNHPLTFGQGTKVEIKR

SEQ ID NO: 393 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNHLNHPLTFGQGTKVEIKR

SEQ ID NO: 394 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNHLNHPLTFGQGTKVEIKR

SEQ ID NO: 395 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLHHPLTFGQGTKVEIKR

SEQ ID NO: 396 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVLHHPLTFGQGTKVEIKR

SEQ ID NO: 397 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNVLHHPLTFGQGTKVEIKR

SEQ ID NO: 398 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNVLHHPLTFGQGTKVEIKR

SEQ ID NO: 399 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNVLHHPLTFGQGTKVEIKR

SEQ ID NO: 400 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVLHHPLTFGQGTKVEIKR

SEQ ID NO: 401 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNVLHHPLTFGQGTKVEIKR

SEQ ID NO: 402 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNVLHHPLTFGQGTKVEIKR

SEQ ID NO: 403 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNVLHHPLTFGQGTKVEIKR

SEQ ID NO: 404 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNVLHHPLTFGQGTKVEIKR

SEQ ID NO: 405 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNVLHHPLTFGQGTKVEIKR

SEQ ID NO: 406 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNVLHHPLTFGQGTKVEIKR

SEQ ID NO: 407 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNVLHHPLTFGQGTKVEIKR

SEQ ID NO: 408 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNVLHHPLTFGQGTKVEIKR

SEQ ID NO: 409 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNVLHHPLTFGQGTKVEIKR

SEQ ID NO: 410 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNVLHHPLTFGQGTKVEIKR

SEQ ID NO: 411 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNHLHHPLTFGQGTKVEIKR

SEQ ID NO: 412 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNHLHHPLTFGQGTKVEIKR

SEQ ID NO: 413 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLADGVPSRFSGSGSGTDFTLTISSLQPEDFATYYCQNHLHHPLTFGQGTKVEIKR

SEQ ID NO: 414 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLADGVPSRFSGSGSGTDFTLTISSLQPEDFATYYCQNHLHHPLTFGQGTKVEIKR

SEQ ID NO: 415 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNHLHHPLTFGQGTKVEIKR

SEQ ID NO: 416 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNHLHHPLTFGQGTKVEIKR

SEQ ID NO: 417 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLADGVPSRFSGSGSGTDFTLTISSLQPEDFATYYCQNHLHHPLTFGQGTKVEIKR

SEQ ID NO: 418 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLADGVPSRFSGSGSGTDFTLTISSLQPEDFATYYCQNHLHHPLTFGQGTKVEIKR

SEQ ID NO: 419 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLAHGVPSRFSGSGSGTDFTLTISSLQPEDFATYYCQNHLHHPLTFGQGTKVEIKR

SEQ ID NO: 420 - protein, 108 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNHLHHPLTFGQGTKVEIKR

SEQ ID NO: 421 - protein, 108 aa, Artificial Sequence
  source: /note=“Description of Artificial Sequence Synthetic polypeptide”
DIQMTQSPSSLSASVGDRVTITCGASENIYHALNWYQQKPGKAPKLLIYGATNLAHGVPSRFSGSGSGTDFTLTISSLQPEDFATYYCQNHLHHPLTFGQGTKVEIKR

SEQ ID NO: 422 - protein, 108 aa, Artificial Sequence
  source: /note=“Description of Artificial Sequence Synthetic polypeptide”
DIQMTQSPSSLSASVGDRVTITCGASENIYGAHNWYQQKPGKAPKLLIYGATNLAHGVPSRFSGSGSGTDFTLTISSLQPEDFATYYCQNHLHHPLTFGQGTKVEIKR

SEQ ID NO: 423 - protein, 108 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNHLHHPLTFGQGTKVEIKR

SEQ ID NO: 424 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLAHGVPSRFSGSGSGTDFTLTISSLQPEDFATYYCQNHLHHPLTFGQGTKVEIKR

SEQ ID NO: 425 - protein, 108 aa, Artificial Sequence
  source: /note=“Description of Artificial Sequence Synthetic polypeptide”
DIQMTQSPSSLSASVGDRVTITCGASENIYHAHNWYQQKPGKAPKLLIYGATNLAHGVPSRFSGSGSGTDFTLTISSLQPEDFATYYCQNHLHHPLTFGQGTKVEIKR

SEQ ID NO: 426 - protein, 108 aa, Artificial Sequence
  source: /note=“Description of Artificial Sequence Synthetic polypeptide”
DIQMTQSPSSLSASVGDRVTITCGAHENIYHAHNWYQQKPGKAPKLLIYGATNLAHGVPSRFSGSGSGTDFTLTISSLQPEDFATYYCQNHLHHPLTFGQGTKVEIKR

SEQ ID NO: 427 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 428 - protein, 330 aa, Artificial Sequence [sequence omitted]

SEQ ID NO: 429 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 430 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 431 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 432 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 433 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALKFHYTQKSLSLSPGK

SEQ ID NO: 434 - protein, 106 aa, Artificial Sequence
  source: /note=“Description of Artificial Sequence Synthetic polypeptide”
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 436 - protein, 214 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 437 - protein, 452 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 452 aa, Artificial Sequence
  source: /note=“Description of Artificial Sequence Synthetic polypeptide”
QVQLVQSGAEVKKPGASVKVSCKASGYIFSNYWHQWVRQAPGQGLEWMGEILPGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 439 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEHLPGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 440 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILHGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGHTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYHFHSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 214 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 214 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNVHNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 214 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLNTHLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 452 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEHLPGSGSTEYTENFKDRVTMTRDTSTSTVYMELSSLRSEDTAVYYCARYFFGSSPNWYFDVWGQGTLVTVSS

SEQ ID NO: 448 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILHGSGSTEYTENFKDRVTMTRDTSTSTVYMELSSLRSEDTAVYYCARYHFGSSPNWYFDVWGQGTLVTVSS

SEQ ID NO: 449 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILHGSGSTEYTENFKDRVTMTRDTSTSTVYMELSSLRSEDTAVYYCARYFFGSSPNWYFDVWGQGTLVTVSS

SEQ ID NO: 450 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFHSSPNWYFDVWGQGTLVTVSS

SEQ ID NO: 451 - protein, 122 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYHFHSSPNWYFDVWGQGTLVTVSS

SEQ ID NO: 452 - protein, 452 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSISTAYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 453 - protein, 214 aa, Artificial Sequence
  source: /note=“Description of Artificial Sequence Synthetic polypeptide”
DIQMTQSPSSLSASVGDRVTITCGAHENIYHALNWYQQKPGKAPKLLIYGATNLAHGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 454 - protein, 214 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVHNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 455 - protein, 108 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVHNTPLTFGQGTKVEIKR

SEQ ID NO: 456 - protein, 214 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLADGVPSRFSGSGSGTDFTLTISSLQPEDFATYYCQNHLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 457 - protein, 214 aa, Artificial Sequence
  source: /note=“Description of Artificial Sequence Synthetic polypeptide”
DIQMTQSPSSLSASVGDRVTITCGAHENIYGALNWYQQKPGKAPKLLIYGATNLAHGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 458 - protein, 214 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVHNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 459 - protein, 108 aa, Artificial Sequence
  source: /note=“Description of Artificial Sequence Synthetic polypeptide”
DIQMTQSPSSLSASVGDRVTITCGAHENIYGAHNWYQQKPGKAPKLLIYGATNLADGVPSRFSGSGSGTDFTLTISSLQPEDFATYYCQNVHNTPLTFGQGTKVEIKR

SEQ ID NO: 460 - protein, 452 aa, Artificial Sequence
  source: /note=“Description of Artificial Sequence Synthetic polypeptide”
QVQLVQSGAEVKKPGASVKVSCKASGYTFSHYWIQWVRQAPGQGLEWMGEILPGSGSTEYTENFKDRVTMTRDTSISTAYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 461 - protein, 452 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEIHPGSGSTEYTENFKDRVTMTRDTSTSTVYMELSSLRSEDTAVYYCARYHFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 462 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYHFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 463 - protein, 214 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLHTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0085095 A1
(43) Pub. Date: Mar. 21, 2019

(54) ANTI-C5 ANTIBODIES WITH ENHANCED PH SWITCH

(71) Applicant: Shire Human Genetic Therapies, Inc., Lexington, MA (US)

(72) Inventors: Madhusudan Natarajan, Lexington, MA (US); Bettina Strack-Logue, Lexington, MA (US); Rizwana Islam, Lexington, MA (US)

(21) Appl. No.: 16/072,696

(22) PCT Filed: Jan. 25, 2017
(86) PCT No.: PCT/US2017/014937

§ 371 Date: Jul. 25, 2018

Related U.S. Application Data
(60) Provisional application No. 62/286,933, filed on Jan. 25, 2016.
(60) Provisional application No. 62/385,059, filed on Sep. 8, 2016.

(51) Int. Cl. C07K 16/36; C07K 16/40; A61P 7/04; C12N 15/62

(52) U.S. Cl. CPC C07K 16/36; C07K 16/40; A61P 7/04; C12N 15/62; C07K 2317/21; C07K 2317/33; C07K 2317/94; C07K 2317/565; C07K 2317/71; C07K 2317/72; C07K 2317/76; C07K 2317/92; C07K 2317/524

(57) ABSTRACT

Multi-specific molecules (e.g., engineered antibodies) that specifically bind to one or more targets, e.g., in a pH sensitive manner, are described, as well as methods of making and using such multi-specific molecules (e.g., engineered antibodies).

### CROSS REFERENCE TO RELATED APPLICATIONS

This application claims priority to U.S. Provisional Patent Application Ser. No. 62/286,933 filed on Jan. 25, 2016 and to 62/385,059 filed on Sep. 8, 2016; the entirety of which is hereby incorporated by reference.

### BACKGROUND

The complement system includes a number of proteins able to interact with pathogens and facilitate pathogen clearance. This system plays important roles in various biological processes such as inflammation, and dysfunction of complement can result in or contribute to disease. At least two pathways known as the “classical pathway” and “alternative pathway” converge on a common set of downstream factors (Complement Components 5 to 9) and events: the terminal complement pathway. Complement Component 5 (“C5”) includes a C5 alpha chain and a C5 beta chain, linked by a disulfide bridge. C5 can be cleaved, producing C5a and C5b cleavage products. C5a is a potent anaphylatoxin. C5b combines with C6, C7, and C8 to form the C5b-8 complex at the surface of a target cell; upon binding of several C9 molecules, the membrane attack complex (MAC, C5b-9, terminal complement complex—TCC) is formed. When sufficient numbers of MACs insert into target cell membranes, the openings they create (MAC pores) mediate rapid osmotic lysis of the target cells.

Various medical conditions resulting from aberrant complement activity could be treated by administration of an antibody or other binding molecule capable of binding C5 and/or inhibiting cleavage of C5. One such antibody is SOLIRIS® (eculizumab). Eculizumab is a humanized monoclonal antibody approved for use in the treatment of certain complement-mediated conditions, including atypical haemolytic uremic syndrome (aHUS) and Paroxysmal Nocturnal Hemoglobinuria (PNH). There is a need in the art for improved anti-C5 antibodies or other C5-binding molecules to better treat patients having complement-related conditions.

### SUMMARY OF INVENTION

The present invention provides, among other things, improved antibodies and/or binding molecules that bind or are capable of binding C5. In various instances, such an antibody or binding molecule is improved in one or more properties as compared to a reference binding molecule, e.g., a reference antibody. In certain instances a reference antibody is an antibody comprising a heavy chain having the amino acid sequence of SEQ ID NO: 1 and a light chain having the amino acid sequence of SEQ ID NO: 2. In certain instances, the reference antibody is eculizumab. The invention is, in part, based on the discovery of a variety of novel histidine substitutions that render pH-dependent binding of the engineered anti-C5 antibodies (also referred to as pH-switch). In particular, as illustrated in the present application including the Examples section, the combination of novel histidine substitution of phenylalanine at position 100 of the heavy chain variable region (amino acid residues 1-122 of SEQ ID NO:1) and histidine substitution of serine at position 26 of the light chain variable region (amino acid residues 1-108 of SEQ ID NO:2), results in unexpectedly superior pH-switch. The discovery of the various novel mutations described herein is truly surprising because extensive histidine scanning has been done on eculizumab. Without wishing to be bound by any particular theory, it is believed that the discovery of these novel mutations is partly due to the use of the IgG1 backbone, instead of the IgG2 or IgG4 backbone commonly used in the prior art, in engineering, screening and/or validating improved anti-C5 monoclonal antibodies. As shown below, an IgG1 backbone further enhances pH-switch as compared to the IgG2 backbone used in eculizumab and, therefore, facilitates identification of novel mutations that otherwise would have been missed using other approaches. Thus, in some embodiments, antibodies described herein include novel mutations that result in pH-switch properties and the antibodies include an IgG1 constant region. Novel mutations described herein provide improved anti-C5 monoclonal antibodies with longer half-life and better processivity, resulting in better safety and efficacy. Thus, the present invention represents a significant advancement in the antibody therapy field for treatment of complement-related conditions.

In one aspect, provided are improved anti-C5 antibodies, or antigen-binding fragments thereof. In certain embodiments, provided are antibodies, or antigen-binding fragments thereof, comprising (i) a light chain variable region comprising an amino acid sequence at least 90% identical to amino acid residues 1-108 of SEQ ID NO:2 and comprising a histidine at position 26; and (ii) a heavy chain variable region comprising an amino acid sequence at least 90% identical to amino acid residues 1-122 of SEQ ID NO:1 and comprising a histidine at position 100. In some embodiments, the heavy chain variable region further comprises a histidine at one or more of positions 27, 31, 34, 51, 52, 53, 57 and 102 of SEQ ID NO:1. In some embodiments, the heavy chain variable region comprises a histidine at position 27 of SEQ ID NO:1. In some embodiments, the heavy chain variable region comprises a histidine at position 31 of SEQ ID NO:1. In some embodiments, the heavy chain variable region comprises a histidine at position 34 of SEQ ID NO:1. In some embodiments, the heavy chain variable region comprises a histidine at position 51 of SEQ ID NO:1. In some embodiments, the heavy chain variable region comprises a histidine at position 52 of SEQ ID NO:1. In some embodiments, the heavy chain variable region comprises a histidine at position 53 of SEQ ID NO:1. In some embodiments, the heavy chain variable region comprises a histidine at position 57 of SEQ ID NO:1. In some embodiments, the heavy chain variable region comprises a histidine at position 102 of SEQ ID NO:1.

In some embodiments, the light chain variable region further comprises a histidine at one or more of positions 31, 33, 56, 91, 92, and 95 of SEQ ID NO:2. In some embodiments, the light chain variable region comprises a histidine at positions 33 and 91 of SEQ ID NO:2. In some embodiments, the light chain variable region comprises a histidine at positions 31 and 56 of SEQ ID NO:2. In some embodiments, the light chain variable region comprises a histidine at positions 56 and 92 of SEQ ID NO:2. In some embodiments, the light chain variable region comprises a histidine at positions 33 and 92 of SEQ ID NO:2. In some embodiments, the light chain variable region comprises a histidine at position 33 of SEQ ID NO:2. In some embodiments, the light chain variable region comprises a histidine at position 95 of SEQ ID NO:2. In some embodiments, the light chain variable region comprises a histidine at position 56 of SEQ ID NO:2.

In some embodiments, the heavy chain variable region comprises a threonine at position 28 of SEQ ID NO:1. In some embodiments, the heavy chain variable region comprises an isoleucine at position 76 of SEQ ID NO:1. In some embodiments, the heavy chain variable region comprises an alanine at position 79 of SEQ ID NO:1.

In some embodiments, the heavy chain variable region comprises an amino acid sequence selected from SEQ ID NO:222, SEQ ID NO:223, or SEQ ID NO:224. In particular embodiments, the heavy chain variable region comprises the amino acid sequence of SEQ ID NO:222. In particular embodiments, the heavy chain variable region comprises the amino acid sequence of SEQ ID NO:223. In particular embodiments, the heavy chain variable region comprises the amino acid sequence of SEQ ID NO:224.

In some embodiments, the light chain variable region comprises an amino acid sequence selected from SEQ ID NO:304, SEQ ID NO:311, SEQ ID NO:312, or SEQ ID NO:328. In particular embodiments, the light chain variable region comprises the amino acid sequence of SEQ ID NO:304. In particular embodiments, the light chain variable region comprises the amino acid sequence of SEQ ID NO:311. In particular embodiments, the light chain variable region comprises the amino acid sequence of SEQ ID NO:312. In particular embodiments, the light chain variable region comprises the amino acid sequence of SEQ ID NO:328.

In certain embodiments, provided are antibodies, or antigen-binding fragments thereof, comprising (i) a light chain variable region comprising an amino acid sequence at least 90% identical to amino acid residues 1-108 of SEQ ID NO:2 and comprising a histidine at position 26 and a histidine at position 56; and/or (ii) a heavy chain variable region comprising an amino acid sequence at least 90% identical to amino acid residues 1-122 of SEQ ID NO:1 and comprising a histidine at position 31 and a histidine at position 100.

In certain embodiments, provided antibody, or antigen-binding fragment thereof, comprising (i) a heavy chain variable domain comprising amino acid residues 1-122 of SEQ ID NO:1 with a histidine at position 100 and one or more additional amino acid substitutions, and (ii) a light chain variable domain comprising amino acid residues 1-108 of SEQ ID NO:2 with a histidine at position 26 and one or more additional amino acid substitutions. In some embodiments, the one or more additional amino acid substitutions in either the heavy chain variable domain or light chain variable domain is no more than one substitution per chain. In some embodiments, the one or more amino acid substitutions in either the heavy chain variable domain or light chain variable domain is no more than two substitutions per chain. In some embodiments, the one or more amino acid substitutions in either the heavy chain variable domain or light chain variable domain is no more than three substitutions per chain. In some embodiments, the one or more amino acid substitutions in either the heavy chain variable domain or light chain variable domain is no more than four substitutions per chain.

In certain embodiments, provided antibodies, or antigen-binding fragments thereof, comprise (i) a light chain variable region comprising an amino acid sequence at least 90% (e.g., at least 92%, 94%, 95%, 96%, 97%, 98%, or 99%) identical to SEQ ID NO:304, SEQ ID NO:311, SEQ ID NO:312, or SEQ ID NO:328; and/or (ii) a heavy chain variable region comprising an amino acid sequence at least 90% (e.g., at least 92%, 94%, 95%, 96%, 97%, 98%, or 99%) identical to SEQ ID NO:222, SEQ ID NO:223, or SEQ ID NO:224.

In certain embodiments, provided antibodies, or antigen-binding fragments thereof, comprise (i) a light chain variable region comprising an amino acid sequence identical to SEQ ID NO:304, SEQ ID NO:311, SEQ ID NO:312, or SEQ ID NO:328; and/or (ii) a heavy chain variable region comprising an amino acid sequence identical to SEQ ID NO:222, SEQ ID NO:223, or SEQ ID NO:224.

In some embodiments, provided antibodies, or antigen-binding fragments thereof, comprise (i) a light chain variable region comprising an amino acid sequence identical to SEQ ID NO:304, and (ii) a heavy chain variable region comprising an amino acid sequence identical to SEQ ID NO:224.

In some embodiments, provided antibodies, or antigen-binding fragments thereof, comprise (i) a light chain variable region comprising an amino acid sequence identical to SEQ SEQ ID NO:311; and (ii) a heavy chain variable region comprising an amino acid sequence identical to SEQ ID NO:222.

In some embodiments, provided antibodies, or antigen-binding fragments thereof, comprise (i) a light chain variable region comprising an amino acid sequence identical to SEQ ID NO:328; and/or (ii) a heavy chain variable region comprising an amino acid sequence identical to SEQ ID NO:223.

In certain embodiments, provided are antibodies, or antigen-binding fragments thereof, comprising (i) a light chain CDR1 comprising the amino acid sequence of SEQ ID NO:279 or SEQ ID NO:279 having one or more amino acid substitutions, (ii) a light chain CDR2 comprising the amino acid sequence of SEQ ID NO:287 or SEQ ID NO:287 having one or more amino acid substitutions, (iii) a light chain CDR3 comprising the amino acid sequence of SEQ ID NO:290 or SEQ ID NO:290 having one or more amino acid substitutions, (iv) a heavy chain CDR1 comprising the amino acid sequence of SEQ ID NO:7 or SEQ ID NO:7 having one or more amino acid substitutions, (v) a heavy chain CDR2 comprising the amino acid sequence of SEQ ID NO:12 or SEQ ID NO:12 having one or more amino acid substitutions, and (vi) a heavy chain CDR3 comprising the amino acid sequence of SEQ ID NO:19 or SEQ ID NO:19 having one or more amino acid substitutions. In some embodiments, the one or more amino acid substitutions in each individual CDR is no more than one substitution. In some embodiments, the one or more amino acid substitutions in each individual CDR is no more than two substitutions. In some embodiments, the one or more amino acid substitutions in each individual CDR is no more than three substitutions.

In certain embodiments, provided are antibodies or antigen-binding fragments thereof comprising (i) a light chain CDR1 comprising the amino acid sequence of SEQ ID NO:279 or SEQ ID NO:279 having one or more amino acid substitutions , (ii) a light chain CDR2 comprising the amino acid sequence of SEQ ID NO:288 or SEQ ID NO:288 having one or more amino acid substitutions, (iii) a light chain CDR3 comprising the amino acid sequence of SEQ ID NO:290 or SEQ ID NO:290 comprising one or more amino acid substitutions, (iv) a heavy chain CDR1 comprising the amino acid sequence of SEQ ID NO:8 or SEQ ID NO:8 having one or more amino acid substitutions, (v) a heavy chain CDR2 comprising the amino acid sequence of SEQ ID NO:12 or SEQ ID NO:12 having one or more amino acid substitutions, and (vi) a heavy chain CDR3 comprising the amino acid sequence of SEQ ID NO:19 or SEQ ID NO:19 having one or more amino acid substitutions. In some embodiments, the one or more amino acid substitutions in each individual CDR is no more than one substitution. In some embodiments, the one or more amino acid substitutions in each individual CDR is no more than two substitutions. In some embodiments, the one or more amino acid substitutions in each individual CDR is no more than three substitutions.

In some embodiments, provided antibodies or antigen-binding fragments thereof comprise (i) the light chain CDR1 comprises the amino acid sequence of SEQ ID NO:279 having a histidine at one or more of positions 8, 10, 11, 31, 33, or 34, (ii) the light chain CDR2 comprises the amino acid sequence of SEQ ID NO:287 having a histidine at one or more of positions 4, 5, or 7, and/or (iii) the light chain CDR3 comprises the amino acid sequence of SEQ ID NO:290 having a histidine at one or more of positions 3, 5, or 6.

In some embodiments, provided antibodies, or antigen-binding fragments thereof, comprises (i) the light chain CDR1 comprises the amino acid sequence of SEQ ID NO:279 having a histidine at positions 8, 10 or 11, (ii) the light chain CDR2 comprises the amino acid sequence of SEQ ID NO:287 having a histidine at positions 4, 5, or 7, and/or (iii) the light chain CDR3 comprises the amino acid sequence of SEQ ID NO:290 having a histidine at positions 3, 5, or 6.

In some embodiments, provided antibodies, or antigen-binding fragments thereof, comprises (i) the light chain CDR1 comprises the amino acid sequence of SEQ ID NO:279 having a histidine at positions 8 (ii) the light chain CDR2 comprises the amino acid sequence of SEQ ID NO:287 having a histidine at position 7, and (iii) the light chain CDR3 comprises the amino acid sequence of SEQ ID NO:290.

In some embodiments, provided antibodies, or antigen-binding fragments thereof, comprising (i) the heavy chain CDR1 comprises the amino acid sequence of SEQ ID NO:7 having a histidine at position 1, 3, or 4, (ii) the heavy chain CDR2 comprises the amino acid sequence of SEQ ID NO:12 having a histidine at position, 2, 3 or 8, and (iii) the heavy chain CDR3 comprises the amino acid sequence of SEQ ID NO:18 having a histidine at positions 2, 4, or 7.

In some embodiments, provided antibodies, or antigen-binding fragments thereof, comprising (i) the heavy chain CDR1 comprises the amino acid sequence of SEQ ID NO:7 having a histidine at position 1, (ii) the heavy chain CDR2 comprises the amino acid sequence of SEQ ID NO:12 and (iii) the heavy chain CDR3 comprises the amino acid sequence of SEQ ID NO:18 having a histidine at positions 2.

In some embodiments, provided antibodies or antigen-binding fragments thereof comprise one or more of (i) the heavy chain CDR1 comprises the amino acid sequence of SEQ ID NO:7 having a histidine at position 1, 3, or 4, (ii) the heavy chain CDR2 comprises the amino acid sequence of SEQ ID NO:12 having a histidine at position, 2, 3 or 8, or (iii) the heavy chain CDR3 comprises the amino acid sequence of SEQ ID NO:18 having a histidine at positions 2, 4, or 7.

In certain embodiments, provided antibodies, or antigen-binding fragments thereof, comprise (i) a light chain CDR1 defined by amino acid sequence of SEQ ID NO:279, SEQ ID NO: 282, SEQ ID NO:283, or 285 (ii) a light chain CDR2 defined by amino acid sequence of SEQ ID NO:287 or SEQ ID NO:288, (iii) a light chain CDR3 defined by amino acid sequence of SEQ ID NO:290, SEQ ID NO:291, or SEQ ID NO:294, (iv) a heavy chain CDR1 defined by amino acid sequence of SEQ ID NO:7, SEQ ID NO:8. or SEQ ID NO:10, (v) a heavy chain CDR2 defined by amino acid sequence of SEQ ID NO:12, SEQ ID NO:13, or SEQ ID NO:15, and (vi) a heavy chain CDR3 defined by amino acid sequence of SEQ ID NO:18, or SEQ ID NO:19.

In some embodiments, a provided antibody, or antigen-binding fragment thereof, comprises (i) a light chain CDR1 defined by the amino acid sequence of SEQ ID NO: 279, (ii) a light chain CDR2 defined by the amino acid sequence of SEQ ID NO:287, (iii) a light chain CDR3 defined by the amino acid sequence of SEQ ID NO:291, (iv) a heavy chain CDR1 defined by the amino acid sequence of SEQ ID NO:7, (v) a heavy chain CDR2 defined by the amino acid sequence of SEQ ID NO:15, and (vi) a heavy chain CDR3 defined by the amino acid sequence of SEQ ID NO:19.

In some embodiments, a provided antibody, or antigen-binding fragment thereof, comprises (i) a light chain CDR1 defined by the amino acid sequence of SEQ ID NO: 282, (ii) a light chain CDR2 defined by the amino acid sequence of SEQ ID NO:288, (iii) a light chain CDR3 defined by the amino acid sequence of SEQ ID NO:290, (iv) a heavy chain CDR1 defined by the amino acid sequence of SEQ ID NO:8, (v) a heavy chain CDR2 defined by the amino acid sequence of SEQ ID NO:12, and (vi) a heavy chain CDR3 defined by the amino acid sequence of SEQ ID NO:19.

In some embodiments, a provided antibody, or antigen-binding fragment thereof, comprises (i) a light chain CDR1 defined by the amino acid sequence of SEQ ID NO: 285, (ii) a light chain CDR2 defined by the amino acid sequence of SEQ ID NO:287, (iii) a light chain CDR3 defined by the amino acid sequence of SEQ ID NO:294, (iv) a heavy chain CDR1 defined by the amino acid sequence of SEQ ID NO:10, (v) a heavy chain CDR2 defined by the amino acid sequence of SEQ ID NO:13, and (vi) a heavy chain CDR3 defined by the amino acid sequence of SEQ ID NO:18.

In some embodiments, provided antibodies or antigen-binding fragments thereof further comprise an IgG constant region. In some embodiments, the IgG constant region is IgG1 In some embodiments, the IgG constant region comprises an alanine at one or both of positions 234 and 235 of a native human IgG constant region, a lysine at position 433 of a native human IgG constant region, and/or a phenylalanine at position 434 of a native human IgG constant region, all according to EU numbering.

In some embodiments, provided antibodies or antigen-binding fragments thereof further comprise a constant region comprising the amino acid sequence of SEQ ID NO:428. In some embodiments, the constant region comprises an alanine at one or both of positions 117 and 118 of SEQ ID NO:428, a lysine at position 316 of SEQ ID NO:428, and/or a phenylalanine at position 317 of SEQ ID NO:428.

In some embodiments, provided antibodies or antigen-binding fragments thereof further comprise a constant region comprising the amino acid sequence of SEQ ID NO: 431. In some embodiments, provided antibodies or antigen-binding fragments thereof further comprise a constant region comprising the amino acid sequence of SEQ ID NO:434.

In some embodiments, provided antibodies comprise a light chain comprising the amino acid sequence of SEQ ID NO: 453 and a heavy chain comprising the amino acid sequence of SEQ ID NO: 452. In some embodiments, provided antibodies comprise a light chain comprising the amino acid sequence of SEQ ID NO:453 and a heavy chain comprising the amino acid sequence of SEQ ID NO:460.

In some embodiments, provided antibodies or antigen-binding fragments thereof bind to complement component human C5. In some embodiments, provided antibodies or antigen-binding fragments thereof inhibit cleavage of C5 into fragments C5a and C5b. In some embodiments, the antibody binds to human C5 at pH 7.4 with an affinity dissociation constant (K_{D}) of about 0.1 nM to about 1 nM (e.g., about 0.1, 0.2, 0.3, 0.4, 0.5, 0.6, 0.7, 0.8, 0.9, or about 1). In some embodiments, the antibody binds to human C5 at pH 7.4 with an affinity dissociation constant (K_{D}) of less than 0.45 nM, less than 0.40 nM, less than 0.35 nM, less than 0.30 nM, or less than 0.25 nM.

In some embodiments, the antibody or antigen-binding fragment thereof binds to human C5 at pH 5.5 with a K_{D} of about 25 to about 200 nM (e.g., about 25, 50, 75, 100, 125, 150, 175, 200). In some embodiments, the antibody or antigen-binding fragment thereof binds to human C5 at pH 5.5 with a K_{D} of greater than 25 nM, greater than 50 nM, greater than 75 nM, greater than 100 nM, greater than 125 nM, greater than 150 nM, or greater than 175 nM.

In some embodiments, the [(K_{D} of the antibody for human C5 at pH 5.5)/(K_{D} of the antibody for human C5 at pH 7.4)] is about 50 to about 750 (e.g., about 50, 100, 150, 200, 250, 300, 350, 400, 450, 500, 550, 600, 650, 700, 750). In some embodiments, the [(K_{D} of the antibody for human C5 at pH 5.5)/(K_{D} of the antibody for human C5 at pH 7.4)] is greater than 50, greater than 100, greater than 150, greater than 200, greater than 250, greater than 300, greater than 350, greater than 400, greater than 450, greater than 500, greater than 550, greater than 600, or greater than 700.

In some embodiments, the off rate of human C5 from the antibody at pH 5.5 is greater than 0.05 s⁻¹. In some embodiments, the off rate of human C5 from the antibody at pH 5.5 is greater than 0.5 s⁻¹. In some embodiments, the off rate of human C5 from the antibody at pH 5.5 is greater than 0.1, 0.15, 0.2, 0.25, 0.3, 0.35, 0.4, or 0.45 s⁻¹.

In one aspect, provided are nucleic acid sequences encoding antibodies or antigen-binding fragments as disclosed herein.

In one aspect, provided are vectors comprising nucleic acid sequences as disclosed herein.

In one aspect, provided are host cells comprising nucleic acid sequences and/or vectors as disclosed herein.

In one aspect, provided are methods of producing antibodies as disclosed herein, or antigen-binding fragments thereof. For example, in certain embodiments, provided are methods comprising culturing host cells as disclosed herein under conditions suitable for expression of the antibodies or antigen-binding fragments.

In one aspect, provided are methods of treating a complement-mediated disease or disorder. For example, in certain embodiments, provided are methods comprising administering to a subject in need thereof an effective amount of an antibody or antigen-binding fragment thereof as disclosed herein.

In one aspect, the present invention provides an engineered multi-specific binding protein comprising an antigen-binding moiety that binds to an antigen, wherein the antigen-binding moiety binds to the antigen with higher affinity at or above a first pH than at or below a second pH; and an FcRn-binding moiety, wherein the FcRn-binding moiety binds to FcRn with lower affinity at or above a first pH than at or below a second pH.

In some embodiments, the first and second pH are an identical pH. In some embodiments, the first pH is higher than the second pH. For example, the first pH may be higher than the second pH by at least 0.1, 0.2, 0.3, 0.4, 0.5, 0.6, 0.7, 0.8, 0.9, 1.0, 1.2, 1.5, 1.8, or 2.0. In some embodiments, the first pH is a neutral pH, e.g., ranging from about 7.0-7.8 (e.g., about 7.0-7.6, about 7.0-7.4, about 7.2-7.6, about 7.2-7.4). In some embodiments, the first pH is about 7.0, 7.1, 7.2, 7.3, 7.4, 7.5, 7.6, 7.7, 7.8. In some embodiments, the first pH is about 7.4.

In some embodiments, the second pH is an acidic pH, e.g., ranging from about 4.8-6.4 (e.g., about 4.8-6.2, about 4.8-6.0, about 5.0-6.4, about 5.0-6.2, about 5.0-6.0, about 5.2-6.4, about 5.2-6.0, about 5.4-6.4, about 5.4-6.2, about 5.4-6.0, about 5.6-6.4, about 5.6-6.2, about 5.6-6.0). In some embodiments, the second pH is about 4.8, 4.9, 5.0, 5.1, 5.2, 5.3, 5.4, 5.5, 5.6, 6.7, 5.8, 5.9, 6.0, 6.1, 6.2, 6.3, or 6.4. In some embodiments, the second pH is about 6.0.

In some embodiments, the FcRn-binding moiety binds to FcRn with affinity of about 0.1 pM to about 100 nM at or below pH about 6.0 and of about 100 pm to about 100 nM at or above pH about 7.4.

In some embodiments, the antigen-binding moiety binds to the antigen with affinity of about 0.1 pM to about 10 nM at or above pH about 7.4, and of about 100 pM and higher to essentially undetectable binding at or below pH about 6.0.

In some embodiments, the FcRn-binding moiety and/or the antigen-binding moiety comprise an amino acid substitution, insertion, or deletion of one or more His, Arg, Lys, Asp, Glu, Ser, Thr, Asn, and/or Gln residues relative to the corresponding wild-type amino acid sequence of the FcRn-binding moiety and/or the antigen-binding moiety, respectively.

In some embodiments, the FcRn-binding moiety and/or the antigen-binding moiety comprise at least one substitution of a His residue for a non-His residue, or a non-His residue for a His residue relative to the corresponding wild-type amino acid sequence.

In some embodiments, the FcRn-binding moiety comprises an Fc domain. In some embodiments, the Fc domain is a modified human IgG Fc domain. In certain embodiments, the modified human IgG Fc domain is a modified Fc domain of human IgG1, IgG2, IgG3, or IgG4. In some embodiments, the modified human IgG Fc domain comprises one or more amino acid substitutions relative to a wild-type human IgG Fc domain at one or more of amino acid residues 252, 254, 256, 309, 311, 428, 433, 434, or 436, numbered according to the EU index as in Kabat.

In some embodiments, the one or more amino acid substitutions comprise H433K and/or N434F substitution.

In some embodiments, the FcRn-binding moiety is a modified albumin domain.

In some embodiments, the antigen-binding moiety binds to complement C3 or C5, an IgG, an IgE, an IgM, CD20, CD40, IL1, IL2, IL4, IL5, IL6, IL8, IL10, IL12, IL21, IL22, IL23, Factor I, II, V, VII, VIII, IX, X, XI, XII, XIII, or cognate receptors.

In some embodiments, the antigen-binding moiety is a Fab, F(ab′)₂, or scFv.

In some embodiments, the multi-specific binding protein comprises at least two antigen-binding moieties.

In some embodiments, the FcRn-binding moiety and the antigen-binding moiety are linked by a peptide linker.

In some embodiments, the multi-specific binding protein is an antibody.

In some embodiments, the first binding moiety binds to the first target with higher affinity at or above a first pH than at or below a second pH by, for example, more than at least about 2, 3, 4, 5, 6, 7, 8, 9, 10, 20, 50, or 100-fold.

In some embodiments, the first binding moiety binds to the first target with lower affinity at or above a first pH than at or below a second pH by, for example, more than at least about 2, 3, 4, 5, 6, 7, 8, 9, 10, 20, 50, or 100-fold.

In some embodiments, the second binding moiety binds to the second target with lower affinity at or above a first pH than at or below a second pH by, for example, more than at least 2, 3, 4, 5, 6, 7, 8, 9, 10, 20, 50, or 100-fold.

In some embodiments, the second binding moiety binds to the second target with higher affinity at or above a first pH than at or below a second pH by, for example, more than at least 2, 3, 4, 5, 6, 7, 8, 9, 10, or 100-fold.

In some embodiments, the first pH and second pH are an identical pH. In some embodiments, the first pH is higher than the second pH by, for example, at least 0.1, 0.2, 0.3, 0.4, 0.5, 1.0, 1.5, or 2.0. In some embodiments, the first pH is a neutral pH (e.g., about 7.4). In some embodiments, the second pH is an acidic pH (e.g., about 6.0).

In some embodiments, a multi-specific binding molecule described herein (e.g. an engineered antibody as described herein) is for use as a medicament.

### DEFINITIONS

A or An: The articles “a” and “an” are used herein to refer to one or to more than one (i.e., to at least one) of the grammatical object of the article. By way of example, “an element” means one element or more than one element.

Affinity: As used herein, the term “affinity” refers to the characteristics of a binding interaction between a binding moiety (e.g., an antigen binding moiety (e.g., variable domain described herein) and/or Fc receptor binding moiety (e.g., FcRn binding moiety described herein)) and a target (e.g., an antigen (e.g., C5) and/or FcR (e.g., FcRn)) and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻M, or higher than about 10⁻⁴ M). In some embodiments, a binding moiety has high affinity for a target at a first pH, has low affinity for the target at a second pH, and has an intermediate affinity for the target at a pH level between the first pH and the second pH.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda.

Binding Moiety: As used herein, a “binding moiety” is any molecule or part of a molecule capable of specifically binding a target, e.g., a target of interest (e.g., an antigen (e.g., C5) and/or FcR (e.g., FcRn)). Binding moieties include, e.g., antibodies, antigen binding fragments thereof, Fc regions or Fc fragments thereof, antibody mimetics, peptides, and aptamers.

Constant region: As used herein, the term “constant region” refers to a polypeptide that corresponds to, or is derived from, one or more constant region immunoglobulin domains of an antibody. A constant region can include any or all of the following immunoglobulin domains: a CH1 domain, a hinge region, a CH2 domain, a CH3 domain (derived from an IgA, IgD, IgG, IgE, or IgM), and a CH4 domain (derived from an IgE or IgM).

Fc region: As used herein, the term “Fc region” refers to a dimer of two “Fc polypeptides”, each “Fc polypeptide” comprising the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or all of the flexible hinge N-terminal to these domains. For IgG, “Fc polypeptide” comprises immunoglobulin domains Cgamma2 (Cγ2) and Cgamma3 (Cγ3) and the lower part of the hinge between Cgamma1 (Cγ1) and Cγ2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, Fc polypeptide comprises immunoglobulin domains Calpha2 (Cα2) and Calpha3 (Cα3) and the lower part of the hinge between Calpha1 (Cα1) and Cα2. An Fc region can be synthetic, recombinant, or generated from natural sources such as IVIG.

Kₐ: As used herein, “Kₐ” refers to an association rate of a particular binding moiety and a target to form a binding moiety/target complex.

K_{d}: As used herein, “K_{d}” refers to a dissociation rate of a particular binding moiety/target complex.

K_{D}: As used herein, “K_{D}” refers to a dissociation constant, which is obtained from the ratio of K_{d} to Kₐ (i.e., K_{d}/Kₐ) and is expressed as a molar concentration (M). K_{D} values can be determined using methods well established in the art, e.g., by using surface plasmon resonance, or using a biosensor system such as a Biacore® system.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

Selective binding: As used herein, “selective binding”, “selectively binds” “specific binding”, or “specifically binds” refers, with respect to a binding moiety and a target, preferential association of a binding moiety to a target and not to an entity that is not the target. A certain degree of non-specific binding may occur between a binding moiety and a non-target. In some embodiments, a binding moiety selectively binds a target if binding between the binding moiety and the target is greater than 2-fold, greater than 5-fold, greater than 10-fold, or greater than 100-fold as compared with binding of the binding moiety and a non-target. In some embodiments, a binding moiety selectively binds a target if the binding affinity is less than about 10⁻⁵ M, less than about 10⁻⁶ M, less than about 10⁻⁷ M, less than about 10⁻⁸ M, or less than about 10⁻⁹ M.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

Target: As used herein, a “target” is any molecule specifically bound by a binding moiety of a multi-specific binding molecule. In some embodiments, a target is an antigen described herein (e.g., C5). In some embodiments, a target is an FcR (e.g., FcRn). The terms “first target” and “second target” are used herein to refer to molecules of two distinct molecular species, rather than two molecules of the same molecular species. For example, in some embodiments, a first target is a serum protein and a second target is FcRn.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

Treatment: As used herein, the term “treatment” (also “treat” or “treating”) refers to any administration of a therapeutic molecule (e.g., an engineered antibody described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF DRAWINGS

Drawings are for illustration purposes only; not for limitation.

FIG. 1A is a schematic illustration depicting an antibody that does not exhibit pH-dependent antigen binding and therefore recycling of the antigen antibody complex.

FIG. 1B is a schematic illustration depicting an antibody that exhibits pH-dependent antigen binding and better binding to the neonatal Fc receptor (FcRn) and thus displays an exemplary scheme to build “better” antibodies.

FIG. 2 is a schematic illustration depicting the basic structure of an anti-C5 antibody.

FIGS. 3A and 3B show representative curves for binding to hC5 over time with the association phase at pH 7.4 and the dissociation phase at pH 7.4 (FIG. 3A) or pH 5.5 (FIG. 3B) for Antibody A and Antibody B, compared against a control antibody (“Control 1”; see Table 6).

FIG. 4 shows representative curves for binding to hC5 over time at pH 7.4, pH 6.0, and pH 5.5 for Antibody B, plotted on a semi-log scale.

FIG. 5A shows representative results of complement-mediated red blood cell (RBC) lysis inhibition assays on engineered anti-C5 antibodies (Antibody A and Antibody B as compared to Control 1), with normalized percentage of RBC lysis plotted against antibody concentration. FIG. 5B shows representative results of inhibition of terminal complement complex (TCC) formation assays on engineered anti-C5 antibodies (Antibody A and Antibody B) as compared to Control 1. Percent formation of the TCC is plotted versus concentration of antibody.

FIGS. 6A and 6B show representative results of experiments to assess antigen depletion mediated by engineered anti-C5 antibodies of the present invention. Mice were injected intravenously with a bolus of human C5 (hC5) and administered either intravenously or subcutaneously engineered anti-C5 antibody (Antibody A). Control 1 was used as a control antibody; some mice were injected with hC5 alone as a control. FIG. 6A shows the total amount of serum human C5 (hC5) protein over time. FIG. 6B shows a subplot of the graph depicted in FIG. 6A.

FIGS. 6C and 6D show representative IgG levels and estimated free C5 levels, respectively, in mice in the antigen-depletion experiment whose results are depicted in FIGS. 6A and 6B.

FIGS. 7A and 7B show representative results of experiments in scid mice implanted with an infusion pump that delivered a constant influx of hC5 for the duration of the experiment and treated with either Antibody A or Control 1 administered either intravenously or subcutaneously. Total hC5 levels in serum were plotted over time. FIG. 7B shows a subplot of the graph shown in FIG. 7A. FIG. 7C shows representative IgG levels in mice from these experiments.

FIGS. 8A, 8B, 8C, 8D, 8E, and 8F show representative binding kinetics of Antibody B at pH 7.4, compared to binding kinetics with an on-rate at pH 7.4 and off-rate at pH 5.5 to endogenous serum C5 from different species (human, cynomolgus monkey, African Green monkey, baboon, CD1 mice, and rhesus monkey, respectively). FIG. 8G shows an overlay of the binding curves at pH 7.4.

FIGS. 9A and 9B show serum IgG levels over time in cynomolgus monkeys administered 1 mg/kg or 30 mg/kg Antibody A or Antibody B, or 30 mg/kg Control 1.

FIG. 10A shows normalized total C5 levels in sera of cynomolgus monkeys administered Antibody B or Control 1 intravenously. FIGS. 10B, 10C, and 10D show pairwise comparisons of the graphs in FIG. 10A.

FIGS. 11A-11J show levels of cytokines released in experiments in which fresh peripheral blood monocytes (PBMCs) were incubated with Antibody B (squares), ZmAb (a negative control shown in downward triangles), or CD3 and CD28 (a positive control shown in circles). FIGS. 11A, 11B, 11C, 11D, 11E, 11F, 11G, 11H, 11I, and 11J show levels of IFNγ, TNFa, IL-4, IL-8, IL-13, IL-1β, IL-2, and IL-12p70, respectively.

FIGS. 12A and 12B show mouse serum chemistry parameters in CD-1 mice treated with Antibody A and Control 1, respectively. Shown are the alanine amino transferase (ALT), aspartate amino transferase (AST), and blood urea nitrogen (BUN) levels in mice administered a single intravenous injection of 10 mg/kg Antibody A or 10 mg/kg Control 1.

FIGS. 13A and 13B show representative curves for binding to hC5 over time for Antibody B compared against BNJ441, an engineered antibody as described in Example 15. The association phase was at pH 7.4 and the dissociation phase was at pH 7.4 (FIG. 13A) or pH 5.5 (FIG. 13B).

FIG. 14 shows representative curves for binding to hC5 over time at pH 5.5 for engineered antibodies with an IgG2 Fc (Antibody D) or IgG1 Fc (Antibody E).

### DESCRIPTION OF INVENTION

The present disclosure is based, in part, on the discovery of engineered antibodies that surprisingly exhibit pH-dependent binding to C5 (e.g., human C5) and/or altered (e.g., increased, e.g., pH dependent) binding to Fc receptor (e.g., FcRn). In some instances, as compared with a reference antibody, engineered antibodies described herein exhibit similar or higher binding affinity for C5 at serum pH, and/or lower binding affinity for C5 at intracellular pH (e.g., endosomal pH). In some instances, as compared with a reference antibody, engineered antibodies described herein exhibit higher binding affinity for FcRn at serum pH and/or higher binding affinity for FcRn at intracellular pH (e.g., endosomal pH). In some aspects, as compared with a reference antibody (e.g., an antibody comprising a heavy chain having the amino acid sequence of SEQ ID NO: 1 and a light chain having the amino acid sequence of SEQ ID NO: 2), engineered anti-C5 antibodies described herein surprisingly exhibit increased affinity for FcRn at a first pH (e.g., at or above about 7.0, e.g., at or about 7.4) and increased affinity for FcRn at a second pH (e.g., at or below about 6.0), without exhibiting reduced affinity for C5 at the first pH In some instances, as compared with a reference antibody, engineered antibodies described herein exhibit similar or lower binding affinity for FcRn at serum pH, and higher binding affinity for FcRn at intracellular pH (e.g., endosomal pH). Without wishing to be bound by theory, it is believed that such pH dependent binding of engineered antibodies to C5 and/or FcRn leads to improved therapeutic properties (e.g., higher antibody serum half-life, higher C5 clearance, lower therapeutic dosage, and/or less frequent dosage regimen), relative to a reference antibody.

The present disclosure also provides a platform to provide improved therapeutic antibodies with improved pharmacology and pharmacodynamics based on a combination of pH-dependent antigen binding and pH-dependent FcRn binding. The present disclosure is, in part, based on the recognition that a better antibody can be engineered to contain both a pH-dependent antigen binding region and a pH-dependent FcRn binding constant region. For example, the antigen binding region can bind an antigen with higher affinity in serum (e.g., at a neutral pH, e.g., at or above pH 7.4) and unbind the antigen in endosomes (e.g., at an acidic pH, e.g., at or below pH 6.0) such that only the antibody is recycled out by FcRn and the antigen will be degraded in lysosomes. This process can improve the “processivity” of the antibody, i.e., the antibody is now free to bind an additional antigen molecule and inactivate it. This process can continue until the antibody is eventually degraded—thus an engineered antibody of the present disclosure can process and inactivate many molecules of antigen throughout its life-time rather than just one. At the same time, the constant region (e.g., Fc domain) of an antibody can also be engineered (i) to enhance the binding to FcRn over that of endogenous IgGs and/or reference antibodies in serum (e.g., at a neutral pH, e.g., at or above pH 7.4) and/or (ii) to enhance pH-dependence of binding to FcRn over that of endogenous IgGs and/or reference antibodies, e.g., exhibiting enhanced binding to FcRn in endosomes (e.g., at an acidic pH, e.g., at or below pH 6.0) relative to endogenous IgG and/or reference antibody binding to FcRn at an acidic pH, as well as in comparison to binding to FcRn in serum (e.g., at a neutral pH, e.g., at or above pH 7.4). Enhancement of the pH-dependent FcRn binding leads to preferential binding of the affinity-enhanced antibody to FcRn over endogenous IgGs and thus leads to a net enhanced recycling of the FcRn-affinity-enhanced antibody, which results in further increased antibody half-life. This increase in half-life is due to enhanced FcRn binding at acidic pH when combined with lower antigen binding at acidic pH relative to serum pH (i.e., an antigen acid-switch) and results in a synergistic increase in the processivity of the antibody that dramatically facilitates degradation of antigen, reduces buildup of antigen-antibody complex, and in cases where the antigen-antibody complex is cleared faster than the antibody itself it significantly increases half-life of antibody. This approach allows highly effective targeting and clearance of antigens, and particularly, “high titer” antigens, including, e.g., IgG, C3, C5, and IgE.

It is further contemplated that the present disclosure may be used to engineer non-antibody proteins or aptamers. For example, the approach described herein can be used to design fusion proteins, chimeric proteins and multi-domain proteins that contain multiple modular pH-dependent binding domains, moieties, or regions for specific targets. For example, such a multi-specific therapeutic protein may contain multiple pH-dependent binding domains to enable binding of multiple targets in serum at physiological pH, unbinding them at endosomal pH for degradation and recycling the therapeutic protein out of the endosomal network by interaction with, e.g., FcRn. Thus, the present invention represents a significant advancement in antibody and other protein therapeutics.

### Antibodies

An engineered antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like, or any combination thereof. The subunit structures and three-dimensional configurations of different classes of antibodies are known in the art.

An antibody can be an immunoglobulin molecule of four polypeptide chains, e.g., two heavy (H) chains and two light (L) chains. A heavy chain can include a heavy chain variable domain and a heavy chain constant domain. A heavy chain constant domain can include CH1 hinge, CH2, CH3, and in some instances CH4 regions. A suitable heavy chain constant region may be derived from any immunoglobulin (e.g., IgA, IgG, or IgE). In some embodiments, a suitable heavy chain constant region may be derived from IgG1, IgG2, or IgG4. In particular embodiments, a suitable heavy chain constant region is derived from IgG1. A light chain can include a light chain variable domain and a light chain constant domain. A light chain constant domain can include either a kappa light chain or a lambda light chain. A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein.

### Engineered Heavy Chain Complementarity Determining Regions

In some embodiments, engineered antibodies described herein can include a heavy chain comprising or consisting of the amino acid sequence of SEQ ID NO:1 (or comprising or consisting of amino acids 1-122 of SEQ ID NO:1). In some embodiments, engineered antibodies described herein include a heavy chain comprising or consisting of the amino acid sequence of SEQ ID NO:1 (or comprising or consisting of amino acids 1-122 of SEQ ID NO:1) and having an amino acid other than Y at position 27 corresponding to SEQ ID NO:1, an amino acid other than N at position 31 corresponding to SEQ ID NO: 1, an amino acid other than I at position 34 of SEQ ID NO:1, an amino acid other than L at position 52 of SEQ ID NO:1, an amino acid other than S at position 57 of SEQ ID NO:1, and/or an amino acid other than F at position 100 corresponding to SEQ ID NO: 1. In some specific instances, an engineered antibody as described herein includes a heavy chain having an amino acid other than Y at position 27 corresponding to SEQ ID NO:1, an amino acid other than N at position 31 corresponding to SEQ ID NO: 1, an amino acid other than I at position 34 of SEQ ID NO:1, an amino acid other than L at position 52 of SEQ ID NO:1, an amino acid other than S at position 57 of SEQ ID NO:1, and/or an amino acid other than F at position 100 corresponding to SEQ ID NO: 1, where the Y at position 27, the N at position 31, the I at position 34, the L at position 52, the S at position 57, and/or the F at position 100 is replaced with an amino acid having a charged side chain. For example the amino acids at these positions can be replaced with an amino acid such as an R, H, K, D, or E amino acid. In some embodiments, one or more of the Y at position 27, the N at position 31, the I at position 34, the L at position 52, the S at position 57, and/or the F at position 100 is/are replaced with an H amino acid.

In various instances of the present invention, an engineered antibody described herein includes a heavy chain CDR1 according to SEQ ID NO:7, 8, 9, or 10. In various instances of the present invention, an engineered antibody described herein includes a heavy chain CDR2 according to SEQ ID NO:12, 13, 14, or 15. In various instances of the present invention, an engineered antibody described herein includes a heavy chain CDR3 according to SEQ ID NO:18 or 19.

As will be understood by those of skill in the art, any such heavy chain CDR sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Heavy Chain Framework Regions

In some embodiments, engineered antibodies described herein can include a heavy chain comprising or consisting of the amino acid sequence of SEQ ID NO:1 (or comprising or consisting of amino acids 1-122 of SEQ ID NO:1) and having an amino acid other than I at position 28 corresponding to SEQ ID NO: 1, an amino acid other than T at position 76 corresponding to SEQ ID NO: 1, and/or an amino acid other than V at position 79 corresponding to SEQ ID NO: 1. In some embodiments, engineered antibodies described herein include a heavy chain comprising or consisting of the amino acid sequence of SEQ ID NO:1 (or comprising or consisting of amino acids 1-122 of SEQ ID NO:1) and having a T at position 28 corresponding to SEQ ID NO:1, an I at position 76 corresponding to SEQ ID NO:1, and/or an A at position 79 corresponding to SEQ ID NO:1.

In various instances of the present invention, an engineered antibody includes a first heavy chain framework region according to SEQ ID NO:3, 4, 5, or 6. In various instances of the present invention, an engineered antibody includes a second heavy chain framework region according to SEQ ID NO:11. In various instances of the present invention, an engineered antibody includes a third heavy chain framework region according to SEQ ID NO:12, 13, 14, or 15. In various instances of the present invention, an engineered antibody includes a fourth heavy chain framework region according to SEQ ID NO: 20.

As will be understood by those of skill in the art, any such heavy chain framework region sequences may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Heavy Chain Constant Domains

In various engineered antibodies described herein, a heavy chain constant domain can be of any class (or subclass). In various engineered antibodies described herein, a heavy chain constant domain can including the amino acid sequence of any of one or more of IgG, IgM, IgA, IgD, or IgE, including subclasses such as IgG1, IgG2, IgG3, IgG4, IgA1, and IgA2. In various instances, a constant domain of engineered antibodies described herein can include a mixture of two or more classes (or subclasses) of immunoglobulin heavy chain constant domain. For instance, an engineered antibody can include a first portion of a constant domain that has a sequence of an immunoglobulin constant domain selected from an IgG, IgM, IgA, IgD, or IgE class constant domain and a second portion of a constant domain that has a sequence of an immunoglobulin constant domain different from the first and selected from an IgG, IgM, IgA, IgD, or IgE class constant domain. In some instances, a constant domain of an engineered antibody described herein can include a mixture of two or more subclasses of a particular class of constant domain, e.g., a first portion of a constant domain that has a sequence of an immunoglobulin constant domain selected from an IgG1, IgG2, IgG3, or IgG4 subclass constant domain and a second portion of a constant domain that has a sequence of an immunoglobulin constant domain different from the first and selected from an IgG1, IgG2, IgG3, or IgG4 subclass constant domain. In some particular embodiments, a constant domain includes all or a portion of an IgG2 constant domain and all or a portion of an IgG4 constant domain.

In some instances, an engineered antibody includes an antibody constant region, Fc region or Fc fragment that exhibits altered binding (as compared to a reference constant region) to one or more Fc receptors (e.g., FcγRI, FcγRIIA, FcγRIIB, FcγRIIIA, FcγRIIIB, FcγRIV, or FcRn receptor). In some embodiments, a constant region, Fc region or Fc fragment is engineered to bind to a target (e.g., an FcRn receptor) in an altered manner (e.g., in a pH sensitive manner (e.g., in a more or less pH sensitive manner) and/or decreased or increased binding) relative to a reference constant region, Fc region or Fc fragment. In some embodiments, an engineered antibody includes an antibody constant region, Fc region or Fc fragment that exhibits decreased binding (as compared to a reference constant region) to one or more Fcγreceptor (e.g., FcγRI, FcγRIIA, FcγRIIB, FcγRIIIA, FcγRIIIB, or FcγRIV). In some embodiments, engineered antibody includes an antibody constant region, Fc region or Fc fragment that exhibits increased binding to the FcRn receptor (as compared to a reference constant region) at serum pH and/or at intracellular pH.

For example, an engineered antibody can include a constant region, Fc region or Fc fragment of an IgG antibody engineered to include an amino acid addition, deletion, or substitution, of one or more of amino acid residues 251-256, 285-290, 308-314, 385-389, and 428-436 (Kabat numbering (Kabat et al., (1991) Sequences of Proteins of Immunological Interest, NIH)). Without wishing to be bound by theory, it is believed that one or more of these constant region, Fc region, or Fc fragment amino acids mediate interaction with an Fc receptor, e.g., FcRn. In some embodiments, one or more of these disclosed amino acids is substituted with histidine, arginine, lysine, aspartic acid, glutamic acid, serine, threonine, asparagine, or glutamine In some embodiments, a non-histidine residue is substituted with a histidine residue. In some embodiments, a histidine residue is substituted with a non-histidine residue.

In some embodiments, an engineered antibody includes a constant region, Fc region or Fc fragment of an IgG antibody having amino acid modifications at one or more of positions 308, 309, 311, 312, and 314, more specifically, having substitutions at one or more of positions 308, 309, 311, 312 and 314 with threonine, proline, serine, aspartic acid and leucine respectively. In some embodiments, residues at one or more of positions 308, 309, and 311 are substituted with isoleucine, proline, and glutamic acid, respectively. In yet other embodiments, residues at one or more of positions 308, 309, 311, 312, and 314, are substituted with threonine, proline, serine, aspartic acid, and leucine, respectively.

In some embodiments, an engineered antibody includes a constant region, Fc region or Fc fragment of an IgG antibody having amino acid modifications at one or more of positions 251, 252, 254, 255, and 256, more specifically, having substitutions at one or more of these positions. In some embodiments, residue 251 is substituted with leucine or arginine, residue 252 is substituted with leucine, tyrosine, phenylalanine, serine, tryptophan or threonine, residue 254 is substituted with threonine or serine, residue 255 is substituted with leucine, glycine, isoleucine or arginine, and/or residue 256 is substituted with serine, phenylalanine, arginine, glutamine, glutamic acid, aspartic acid, alanine, asparagine or threonine. In some embodiments, residue 251 is substituted with leucine, residue 252 is substituted with tyrosine or leucine, residue 254 is substituted with threonine or serine, and/or residue 255 is substituted with arginine. In yet other embodiments, residue 252 is substituted with phenylalanine and/or residue 256 is substituted with aspartic acid. In some embodiments, residue 251 is substituted with leucine, residue 252 is substituted with tyrosine, residue 254 is substituted with threonine or serine, and/or residue 255 is substituted with arginine.

In some embodiments, an engineered antibody includes a constant region, Fc region or Fc fragment of an IgG antibody having amino acid modifications at one or more of positions 428, 433, 434, 435, and 436, more specifically, having substitutions at one or more of these positions. In some embodiments, residue 428 is substituted with methionine, threonine, leucine, phenylalanine, or serine, residue 433 is substituted with lysine, arginine, serine, isoleucine, proline, glutamine, or histidine, residue 434 is substituted with phenylalanine, tyrosine, or histidine, residue 435 is substituted with tyrosine, and/or residue 436 is substituted with histidine, asparagine, arginine, threonine, lysine, methionine, or threonine. In some embodiments, residues at one or more positions 433, 434, 435, and 436 are substituted with lysine, phenylalanine, tyrosine, and histidine, respectively. In some embodiments, residue 428 is substituted with methionine and/or residue 434 is substituted with tyrosine.

In some embodiments, an engineered antibody includes a constant region, Fc region or Fc fragment of an IgG antibody having amino acid modifications at one or more of positions 385, 386, 387, and 389, more specifically, having substitutions at one or more of these positions. In some embodiments, residue 385 is substituted with arginine, aspartic acid, serine, threonine, histidine, lysine, or alanine, residue 386 is substituted with threonine, proline, aspartic acid, serine, lysine, arginine, isoleucine, or methionine, residue 387 is substituted with arginine, histidine, serine, threonine, alanine, or proline and/or residue 389 is substituted with proline or serine. In some embodiments, residues at one or more of positions 385, 386, 387, and 389 are substituted with arginine, threonine, arginine, and proline, respectively. In some embodiments, residues at one or more of positions 385, 386, and 389 are substituted with aspartic acid, proline, and serine, respectively.

In some embodiments, an engineered antibody includes a constant region, Fc region or Fc fragment of an IgG antibody having one or more of the following substitutions: leucine at residue 251, tyrosine or leucine at residue 252, threonine or serine at residue 254, arginine at residue 255, threonine at residue 308, proline at residue 309, serine at residue 311, aspartic acid at residue 312, leucine at residue 314, arginine at residue 385, threonine at residue 386, arginine at residue 387, proline at residue 389, methionine at residue 428, lysine at residue 433, phenylalanine or tyrosine at residue 434, tyrosine at position 435, and/or tyrosine at position 436. Additional amino acid substitutions that can be included in a constant region, Fc region or Fc fragment include those described in, e.g., U.S. Pat. Nos. 6,277,375; 8,012,476; and 8,163,881.

In some embodiments, an engineered antibody described herein includes a heavy chain constant domain that include the Ala-Ala mutation described in, e.g., PCT Publication nos. WO 94/28027 and WO 98/47531; and Xu et al. (2000) Cell Immunol 200:16-26. Thus, in some embodiments, an engineered antibody with one or more mutations within the heavy chain constant region including the Ala-Ala mutation has reduced or no effector function. According to these embodiments, the constant region of an engineered antibody described herein can comprise a substitution to an alanine at position 234 and/or a mutation to an alanine at position 235 (EU numbering).

In some embodiments, an engineered antibody described herein includes a heavy chain constant domain comprising or consisting of the amino acid sequence of SEQ ID NO:427. In some embodiments, an engineered antibody described herein includes a heavy chain constant domain comprising or consisting of the amino acid sequence of SEQ ID NO:427 and having an amino acid other than H at position 284 of SEQ ID NO:427. In some embodiments, an engineered antibody described herein includes a heavy chain constant domain comprising or consisting of the amino acid sequence of SEQ ID NO:427 and having an amino acid other than N at position 285 of SEQ ID NO:427. In some embodiments, an engineered antibody described herein includes a heavy chain constant domain comprising or consisting of the amino acid sequence of SEQ ID NO:427 and having an amino acid other than H at position 284 of SEQ ID NO:427 and having an amino acid other than N at position 285 of SEQ ID NO:427. In some embodiments, an engineered antibody described herein includes a heavy chain constant domain comprising or consisting of the amino acid sequence of SEQ ID NO:427 and having a K at position 284 of SEQ ID NO:427 and/or having an F at position 285 of SEQ ID NO:427.

In some embodiments, an engineered antibody described herein includes a heavy chain constant domain comprising or consisting of the amino acid sequence of SEQ ID NO:428. In some embodiments, an engineered antibody described herein includes a heavy chain constant domain comprising or consisting of the amino acid sequence of SEQ ID NO:428 and having an amino acid other than L at position 117 of SEQ ID NO:428. In some embodiments, an engineered antibody described herein includes a heavy chain constant domain comprising or consisting of the amino acid sequence of SEQ ID NO:428 and having an amino acid other than L at position 118 of SEQ ID NO:428. In some embodiments, an engineered antibody described herein includes a heavy chain constant domain comprising or consisting of the amino acid sequence of SEQ ID NO:428 and having an amino acid other than L at position 117 of SEQ ID NO:428 and having an amino acid other than L at position 118 of SEQ ID NO:428. In some embodiments, an engineered antibody described herein includes a heavy chain constant domain comprising or consisting of the amino acid sequence of SEQ ID NO:428 and having an amino acid other than H at position 316 of SEQ ID NO:428. In some embodiments, an engineered antibody described herein includes a heavy chain constant domain comprising or consisting of the amino acid sequence of SEQ ID NO:428 and having an amino acid other than N at position 317 of SEQ ID NO:428. In some embodiments, an engineered antibody described herein includes a heavy chain constant domain comprising or consisting of the amino acid sequence of SEQ ID NO:428 and having an amino acid other than H at position 316 of SEQ ID NO:428 and having an amino acid other than N at position 317 of SEQ ID NO:428. In some embodiments, an engineered antibody described herein includes a heavy chain constant domain comprising or consisting of the amino acid sequence of SEQ ID NO:429, 430, or 431.

In some embodiments, an engineered antibody described herein includes a heavy chain constant domain comprising or consisting of the amino acid sequence of SEQ ID NO:432. In some embodiments, an engineered antibody described herein includes a heavy chain constant domain comprising or consisting of the amino acid sequence of SEQ ID NO:432 and having an amino acid other than H at position 312 of SEQ ID NO:432. In some embodiments, an engineered antibody described herein includes a heavy chain constant domain comprising or consisting of the amino acid sequence of SEQ ID NO:432 and having an amino acid other than N at position 313 of SEQ ID NO:432. In some embodiments, an engineered antibody described herein includes a heavy chain constant domain comprising or consisting of the amino acid sequence of SEQ ID NO:432 and having an amino acid other than H at position 312 of SEQ ID NO:432 and having an amino acid other than N at position 313 of SEQ ID NO:432. In some embodiments, an engineered antibody described herein includes a heavy chain constant domain comprising or consisting of the amino acid sequence of SEQ ID NO:433. As will be understood by those of skill in the art, any such heavy chain constant domain sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Light Chain Complementarity Determining Regions

In some embodiments, engineered antibodies described herein can include a light chain comprising or consisting of the amino acid sequence of SEQ ID NO:2 (or comprising or consisting of amino acids 1-108 of SEQ ID NO:2). In some embodiments, engineered antibodies described herein can include a light chain comprising or consisting of the amino acid sequence of SEQ ID NO:2 (or comprising or consisting of amino acids 1-108 of SEQ ID NO:2) and having an amino acid other than S at position 26 of SEQ ID NO:2, an amino acid other than G at position 31 of SEQ ID NO:2, an amino acid sequence other than L at position 33 of SEQ ID NO:2, an amino acid other than D at position 56 of SEQ ID NO:2, an amino acid other than V at position 91 of SEQ ID NO:2, and/or an amino acid other than T at position 94 of SEQ ID NO:2. In some embodiments, engineered antibodies described herein can include a light chain comprising or consisting of the amino acid sequence of SEQ ID NO:2 (or comprising or consisting of amino acids 1-108 of SEQ ID NO:2) and having an amino acid other than S at position 26 of SEQ ID NO:2, an amino acid other than G at position 31 of SEQ ID NO:2, an amino acid sequence other than L at position 33 of SEQ ID NO:2, an amino acid other than D at position 56 of SEQ ID NO:2, an amino acid other than V at position 91 of SEQ ID NO:2, and/or an amino acid other than T at position 94 of SEQ ID NO:2, where the S at position 26, G at position 31, L at position 33, D at position 56, V at position 91, and/or T at position 94 is replaced with an amino acid such as an R, H, K, D, or E amino acid. In some embodiments, one or more of the S at position 26 of SEQ ID NO:2, the G at position 31 of SEQ ID NO:2, the L at position 33 of SEQ ID NO:2, the D at position 56 of SEQ ID NO:2, the V at position 91 of SEQ ID NO:2, and/or the T at position 94 of SEQ ID NO:2 is/are replaced with an H amino acid.

In some embodiments, an engineered antibody described herein includes a light chain CDR1 according to SEQ ID NO:278, 279, 280, 281, 282, 283, 284, or 285. In some embodiments, an engineered antibody described herein includes a light chain CDR2 according to SEQ ID NO:287 or 288. In some embodiments, an engineered antibody described herein includes a light chain CDR3 according to SEQ ID NO:290, 291, 292, 293, 294, 295, 296, or 297.

As will be understood by those of skill in the art, any such light chain CDR sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Light Chain Framework Regions

In some embodiments, an engineered antibody described herein can include a light chain comprising or consisting of one or more light chain framework sequences present in a reference antibody (e.g., a light chain framework region included in SEQ ID NO:2). As those of skill in the art will be aware, a typical full-length light chain includes four framework regions intercalated by three CDRs. Each of any such four framework regions may be independently selected from various sources, including the sequence of a light chain known in the art, e.g., a reference antibody comprising a heavy chain having the amino acid sequence of SEQ ID NO: 1 and a light chain having the amino acid sequence of SEQ ID NO: 2.

In some embodiments, engineered antibodies described herein can include a first light chain framework region according to SEQ ID NO:277. In some embodiments, engineered antibodies described herein can include a second light chain framework region according to SEQ ID NO:286. In some embodiments, engineered antibodies described herein can include a third light chain framework region according to SEQ ID NO:289. In some embodiments, engineered antibodies described herein can include a fourth light chain framework region according to SEQ ID NO:298.

As will be understood by those of skill in the art, any such light chain framework region sequences may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Engineered Light Chain Constant Domains

In some embodiments, an engineered antibody described herein includes a light chain that includes any light chain constant domain sequence, e.g., a constant sequence of a light chain known to those of skill in the art. As those of skill in the art will be aware, a light chain constant domain may be a kappa light chain constant domain or a lambda light chain constant domain. In certain embodiments, the constant domain of a light chain as disclosed herein is a kappa light chain constant domain. For instance, a light chain can include a constant domain present in a reference antibody comprising a heavy chain having the amino acid sequence of SEQ ID NO: 1 and a light chain having the amino acid sequence of SEQ ID NO: 2.

In various instances, an engineered antibody described herein includes a light chain constant domain comprising or consisting of the amino acid sequence of SEQ ID NO:434. As will be understood by those of skill in the art, any such light chain constant domain sequence may be readily combined, e.g., by techniques of molecular biology, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

### Exemplary Engineered Antibodies

Engineered antibodies can include various heavy chains and light chains described herein. In some embodiments, an engineered antibody can include two heavy chains and light chains. In various instances, the present disclosure encompasses an antibody including at least one heavy chain and/or light chain as disclosed herein, at least one heavy chain and/or light chain framework domain as disclosed herein, at least one heavy chain and/or light chain CDR domain as disclosed herein, and/or any heavy chain and/or light chain constant domain as disclosed herein.

In various instances, an engineered antibody disclosed herein is a homodimeric monoclonal antibody. In various instances, an engineered antibody disclosed herein is a heterodimeric antibody. In various instances, an engineered antibody is, e.g., a typical antibody or a diabody, triabody, tetrabody, minibody, maxibody, tandab, DVD, BiTe, scFv, TandAb scFv, Fab, Fab₂, Fab₃, F(ab′)₂, or the like, or any combination thereof.

Certain engineered antibodies of the disclosure include at least one (e.g., at least 2, 3, 4, 5, 6, 7, 8, 9, or 10) amino acid substitutions within a heavy chain at one or more of the following positions (relative to SEQ ID NO:1): Y27, I28, N31, I34, L52, S57, F100. In some embodiments, an engineered antibody includes a heavy chain having an amino acid substitution at position F100 and one or more (e.g., 1, 2, 3, 4, 5, 6, 7, 8, 9, or 10) additional amino acid substitutions within a heavy chain and/or a light chain. Exemplary combinations of heavy chain amino acid positions (relative to SEQ ID NO:1) that can be substituted are depicted in Table 1.

TABLE 1
Exemplary Heavy Chain Substitutions

| Y27 | I28 | N31 | I34 | I51 | L52 | P53H | S57 | F100 | G102H |
|---|---|---|---|---|---|---|---|---|---|
| | | | | | | | | X | |
| | X | | | | | | | X | |
| | | | | | | | X | X | |
| | X | | | | | | X | X | |
| | | | | | X | | X | X | |
| | X | | | | X | | X | X | |
| | | | X | | X | | X | X | |
| | X | | X | | X | | X | X | |
| | | X | X | | X | | X | X | |
| | X | X | X | | X | | X | X | |
| X | | X | X | | X | | X | X | |
| X | X | X | X | | X | | X | X | |
| | | | X | | | | X | X | |
| | X | | X | | | | X | X | |
| | | X | X | | | | X | X | |
| | X | X | X | | | | X | X | |
| X | | X | X | | | | X | X | |
| X | X | X | X | | | | X | X | |
| | | X | | | | | X | X | |
| | X | X | | | | | X | X | |
| X | | X | | | | | X | X | |
| X | X | X | | | | | X | X | |
| X | | | | | | | X | X | |
| X | X | | | | | | X | X | |
| | | | | | X | | | X | |
| | X | | | | X | | | X | |
| | | | X | | X | | | X | |
| | X | | X | | X | | | X | |
| | | X | X | | X | | | X | |
| | X | X | X | | X | | | X | |
| X | | X | X | | X | | | X | |
| X | X | X | X | | X | | | X | |
| | | X | | | X | | | X | |
| | X | X | | | X | | | X | |
| X | | X | | | X | | | X | |
| X | X | X | | | X | | | X | |
| X | | | | | X | | | X | |
| X | X | | | | X | | | X | |
| | | | X | | | | | X | |
| | X | | X | | | | | X | |
| | | X | X | | | | | X | |
| | X | X | X | | | | | X | |
| X | | X | X | | | | | X | |
| X | X | X | X | | | | | X | |
| X | | | X | | | | | X | |
| X | X | | X | | | | | X | |
| | | X | | | | | | X | |
| | X | X | | | | | | X | |
| X | | X | | | | | | X | |
| X | X | X | | | | | | X | |
| X | | | | | | | | X | |
| X | X | | | | | | | X | |
| | | | | | | X | | X | |
| | | | | | | | | X | X |
| | | | | X | | | | X | |
| | | | | | | X | | X | |

In some embodiments, a heavy chain described herein includes at least one heavy chain variable domain comprising or consisting of the amino acid sequence of any one of SEQ ID NOs:21-276.

In some embodiments, a heavy chain described herein includes at least one heavy chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID NOs:21-276.

In some embodiments, an engineered antibody described herein includes at least one heavy chain variable domain comprising or consisting of the amino acid sequence of any one of SEQ ID NOs:21-276.

In some embodiments, an engineered antibody described herein includes at least one heavy chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID NOs:21-276.

In some embodiments, an engineered antibody described herein includes (i) at least one heavy chain variable domain comprising or consisting of the amino acid sequence of any one of SEQ ID NOs:21-276, and (ii) at least one heavy chain constant domain comprising or consisting of the amino acid sequence of any one of SEQ ID NOs: 427-433.

In some embodiments, an engineered antibody described herein includes (i) at least one heavy chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to any one of SEQ ID NOs: 21-276, and (ii) at least one heavy chain constant domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID NOs: 427-433.

Certain engineered antibodies of the disclosure include at least one (e.g., at least 2, 3, 4, 5, 6, 7, 8, 9, or 10) amino acid substitution within a light chain at one or more of the following positions (relative to SEQ ID NO:2): S26, E27, N28, G31, L33, N53, D56, V91, T94. In some embodiments, an engineered antibody includes a light chain having an amino acid substitution at position S26 and one or more (e.g., 1, 2, 3, 4, 5, 6, 7, 8, 9, or 10) additional amino acid substitutions within a light chain and/or a heavy chain. Exemplary combinations of light chain amino acid positions (relative to SEQ ID NO:2) that can be substituted are depicted in Table 2.

TABLE 2
Exemplary Light Chain Substitutions

| S26 | G31 | L33 | D56 | V91 | L92 | N93 | T94 | P95 |
|---|---|---|---|---|---|---|---|---|
| X | | | | | | | | X |
| X | X | | | | | | | |
| X | X | X | | | | | | |
| X | X | X | X | | | | | |
| X | X | X | X | X | | | | |
| X | X | X | X | X | | | X | |
| X | X | | X | | | | | |
| X | X | | X | X | | | | |
| X | X | | X | X | | | X | |
| X | | | | X | | | | |
| X | | | | X | | | X | |
| X | | | | | | | X | |
| X | | X | | | | | | |
| X | | X | X | | | | | |
| X | | X | X | X | | | | |
| X | | X | X | X | | | X | |
| X | | X | | X | | | | |
| X | | X | | X | | | X | |
| X | | X | | | | | X | |
| X | | | X | | | | | |
| X | | | X | X | | | | |
| X | | | X | X | | | X | |
| X | | | X | | | | X | |
| X | | | | X | | | | |
| X | | | | X | | | X | |
| X | | | | | | | X | |
| X | | | | | X | | | |
| X | | | | | | X | | |

In some embodiments, a light chain described herein includes at least one light chain variable domain comprising or consisting of the amino acid sequence of any one of SEQ ID NOs:299-426.

In some embodiments, a light chain described herein includes at least one light chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID NOs:299-426.

In some embodiments, an engineered antibody described herein includes at least one light chain variable domain comprising or consisting of the amino acid sequence of any one of SEQ ID NOs:299-426.

In some embodiments, an engineered antibody described herein includes at least one light chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of any one of SEQ ID NOs:299-426.

In some embodiments, an engineered antibody described herein includes (i) at least one light chain variable domain comprising or consisting of the amino acid sequence of any one of SEQ ID NOs:299-426, and (ii) at least one light chain constant domain comprising or consisting of the amino acid sequence of SEQ ID NO:434.

In some embodiments, an engineered antibody described herein includes (i) at least one light chain variable domain comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to any one of SEQ ID NOs:299-426, and (ii) at least one light chain constant region comprising or consisting of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% homology or identity to the amino acid sequence of SEQ ID NO: 434.

In some embodiments, an engineered antibody described herein includes a heavy chain CDR as disclosed herein and a light chain CDR as disclosed herein. Accordingly, in certain instances, an engineered antibody described herein includes one heavy chain or two heavy chains including CDR1 comprising or consisting of the amino acid sequence of SEQ ID NO:7, 8, 9, or 10, and/or CDR2 comprising or consisting of the amino acid sequence of SEQ ID NO:12, 13, 14, or 15, and/or CDR3 comprising or consisting of the amino acid sequence of SEQ ID NO:18 or 19, and/or one light chain or two light chains including CDR1 comprising or consisting of the amino acid sequence of SEQ ID NO:278, 279, 280, 281, 282, 283, 284, or 285, and/or a CDR2 comprising or consisting of the amino acid sequence of SEQ ID NO:287 or 288, and/or a CDR3 comprising or consisting of the amino acid sequence of SEQ ID NO:290, 291, 292, 293, 294, 295, 296, or 297. In various embodiments, an antibody as disclosed herein includes two light chains and two heavy chains. In various embodiments, an antibody includes two light chains having the same amino acid sequence. In various embodiments, an antibody includes two heavy chains having the same amino acid sequence. In various embodiments, an antibody includes two light chains having the same amino acid sequence and two heavy chains having the same amino acid sequence. Any of one or more heavy chains may include a constant domain comprising or consisting of the amino acid sequence of SEQ NO:427, 428, 429, 430, 431, 432, or 433. Any of one or more light chains may include a constant domain comprising or consisting of the amino acid sequence of SEQ ID NO:434.

In some embodiments, an engineered antibody described herein includes a heavy chain variable domain as disclosed herein and a light chain variable domain as disclosed herein. Accordingly, in certain instances, an engineered antibody includes (i) one heavy chain or two heavy chains including a heavy chain variable domain comprising or consisting of the amino acid sequence of any one of SEQ ID NOs:21-276 and/or (ii) one light chain or two light chains including a light chain variable domain comprising or consisting of the amino acid sequence of any one of SEQ ID NOs:299-426. In various embodiments, an engineered antibody as disclosed herein includes two light chains and two heavy chains. In various embodiments, an engineered antibody includes two light chains having the same amino acid sequence. In various embodiments, an engineered antibody includes two heavy chains having the same amino acid sequence. In various embodiments, an engineered antibody includes two light chains having the same amino acid sequence and two heavy chains having the same amino acid sequence. Any of one or more heavy chains may include a constant domain comprising or consisting of the amino acid sequence of SEQ NO:427, 428, 429, 430, 431, 432, or 433. Any of one or more light chains may include a constant domain comprising or consisting of the amino acid sequence of SEQ ID NO:434.

In various embodiments, an engineered antibody described herein includes a heavy chain as disclosed herein and a light chain as disclosed herein. Accordingly, in certain instances, an engineered antibody of the present disclosure includes (1) one heavy chain or two heavy chains including (i) at least one heavy chain variable domain comprising or consisting of the amino acid sequence of any one of SEQ ID NOs:21-276, and (ii) at least one heavy chain constant domain comprising or consisting of the amino acid sequence of any one of SEQ ID NOs: 427-433, and/or (2) one light chain or two light chains including (i) at least one light chain variable domain comprising or consisting of the amino acid sequence of any one of SEQ ID NOs:299-426, and (ii) at least one light chain constant domain comprising or consisting of the amino acid sequence of SEQ ID NO:434. In various embodiments, an engineered antibody as disclosed herein includes two light chains and two heavy chains. In various embodiments, an engineered antibody includes two light chains having the same amino acid sequence. In various embodiments, an engineered antibody includes two heavy chains having the same amino acid sequence. In various embodiments, an engineered antibody includes two light chains having the same amino acid sequence and two heavy chains having the same amino acid sequence. Any of one or more heavy chains may include a constant domain comprising or consisting of the amino acid sequence of SEQ NO:427, 428, 429, 430, 431, 432, or 433. Any of one or more light chains may include a constant domain comprising or consisting of the amino acid sequence of SEQ ID NO:434.

### Engineered Fusion Proteins

In some embodiments, the disclosure provides fusion proteins comprising (i) one or more antigen-binding regions described herein (e.g., antigen-binding region of immunoglobulin, heavy chain antibody, light chain antibody, LRR-based antibody, or other protein scaffold with antibody-like properties, as well as other antigen binding moiety known in the art, including, e.g., a Fab, Fab′, Fab′2, Fab₂, Fab₃, F(ab′)₂, Fd, Fv, Feb, scFv, SMIP, antibody, diabody, triabody, tetrabody, minibody, maxibody, tandab, DVD, BiTe, TandAb, or the like), e.g., one or more variable domains described herein, or portion thereof (e.g., one or more CDRs described herein), and (ii) one or more additional polypeptides. For example, albumin is an abundant serum protein that is protected from degradation by pH-dependent recycling mediated by interaction with FcRn. In some embodiments, one or more variable domains or engineered antibodies as described herein, or portion thereof (e.g., one or more CDRs described herein) is fused to albumin, a portion thereof (such as a portion of albumin that binds to an FcRn), and/or an engineered variant of albumin that binds to FcRn with improved affinity. In other instances, one or more variable domains or engineered antibodies as described herein, or portion thereof (e.g., one or more CDRs described herein) is fused to a polypeptide that binds to albumin to form a fusion protein-albumin complex, which can in turn bind to an FcRn. In some embodiments, the polypeptide that binds to albumin is a single chain variable fragment (scFv). The albumin or portion thereof can include a mutation of one or more amino acids that can modify its binding to an FcRn. Such mutations are known in the art (see, e.g., Andersen et al., Nature Communications 3:610 doi: 10.1038/nocmms1607 (2012)).

In other instances, one or more variable domains or engineered antibodies described herein, or portion thereof (e.g., one or more CDRs described herein) is fused to transferrin. Transferrin is recycled by binding to a transferrin receptor (see, e.g., Widera et al., Adv. Drug Deliv. Rev. 55:1439-66 (2003)).

In some embodiments, the disclosure provides fusion proteins comprising one or more variable domains or engineered antibodies as described herein, or portion thereof, and one or more additional polypeptides and/or scFvs that bind to FcRn.

Exemplary sequences of engineered heavy chain and light chain CDRs, variable regions, constant regions, framework regions, and full length are shown in Table 12.

### Nucleotide Sequences

The present disclosure includes nucleotide sequences encoding one or more heavy chains, heavy chain variable domains, heavy chain framework regions, heavy chain CDRs, heavy chain constant domains, light chains, light chain variable domains, light chain framework regions, light chain CDRs, light chain constant domains, or other immunoglobulin-like sequences, antibodies, or binding molecules disclosed herein. In various instances, such nucleotide sequences may be present in a vector. In various instances such nucleotides may be present in the genome of a cell, e.g., a cell of a subject in need of treatment or a cell for production of an antibody, e.g. a mammalian cell for production of a an antibody.

### PEGylation

In certain embodiments, an engineered antibody as described herein can be PEGylated to include mono- or poly-(e.g., 2-4) PEG moieties. Such PEGylated antibodies may display increased half-life in comparison to a non-PEGylated reference antibody, e.g., an antibody having the same amino acid sequence but different, a different amount of, or no PEGylation.

PEGylation can be carried out by any suitable reaction known in the art. Methods for preparing a PEGylated protein can generally include (a) reacting a polypeptide with polyethylene glycol (such as a reactive ester or aldehyde derivative of PEG) under conditions whereby the polypeptide becomes attached to one or more PEG groups; and (b) obtaining the reaction product(s). In general, the conditions for the reactions can be determined case by case based on known parameters and the desired result.

There are a number of PEG attachment methods available to those skilled in the art. For example, the step of PEGylating a multi-specific binding molecule described herein can be carried out via an acylation reaction or an alkylation reaction with a reactive polyethylene glycol molecule.

pH-Dependent Binding of Engineered Antibody with C5 and/or Fc Receptor

Engineered antibodies described herein exhibit pH-dependency, or enhanced pH dependency, in affinity for C5 (e.g., mediated by one or more variable domains described herein), and/or altered (e.g., increased, e.g., pH dependent) affinity for FcRn (e.g., mediated by one or more constant domains described herein). For example, in some embodiments an antibody capable of binding C5, or a variable domain capable of binding C5, binds C5 with higher affinity at a serum pH (e.g., at a neutral pH or at a pH above 7.4) than at a compartmental (e.g., endosomal) pH (e.g., at an acidic pH or at a pH equal to or less than pH 6.0). In various instances in which C5 is bound by an antibody having pH-dependent C5 binding, a transition of pH from serum pH to compartmental pH (e.g., from serum to endosome) facilitates separation of C5 and antibody (i.e., “unbinding”) at compartmental pH and/or in a particular compartment, e.g., endosome. In various instances, such pH-dependent binding can mediate antibody recycling and/or C5 degradation. In particular instances, a transition from serum pH to compartmental pH (e.g., from serum to endosome) facilitates separation of C5 and antibody (i.e., “unbinding”) at the compartmental pH and/or in a particular compartment, e.g., endosome, such that the antibody is recycled out by FcRn and the antigen is degraded in a lysosome. In some such instances, the pH-dependency of C5 binding improves the “processivity” of the antibody at least in that, upon recycling, the antibody is returned to serum and is free to bind target circulating C5. In some instances, recycling of an antibody that displays pH-dependent C5 binding can continue until the antibody eventually degrades or is degraded, by which time a single antibody molecule may have bound and mediated the inactivation of a plurality of C5 molecules, rather than just one.

In certain embodiments, an engineered antibody disclosed herein includes a constant domain (e.g., an Fc domain) displaying increased affinity relative to control (e.g., a reference antibody comprising a heavy chain having the amino acid sequence of SEQ ID NO: 1 and a light chain having the amino acid sequence of SEQ ID NO: 2), for an Fc receptor, such as FcRn. In some embodiments, such increased affinity relative to control is at a pH value for serum (e.g., pH greater than 6.8, 6.9, 7.0, 7.1, 7.2, 7.3, 7.4, 7.5, 7.6, 7.7, 7.8, 7.9, 8.0, 8.1, 8.2, or greater). In some embodiments, such increased affinity relative to control is at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower). In certain embodiments, an engineered antibody disclosed herein includes a constant domain (e.g., an Fc domain) displaying pH-dependency (or enhanced pH dependency relative to control, e.g., a reference antibody comprising a heavy chain having the amino acid sequence of SEQ ID NO: 1 and a light chain having the amino acid sequence of SEQ ID NO: 2), in affinity for an Fc receptor, such as FcRn. The neonatal Fc receptor (FcRn) is a MHC class I like molecule that functions to protect IgG and albumin from catabolism, mediates transport of IgG across epithelial cells, and is involved in antigen presentation by professional antigen presenting cells. IgG antibody subtypes exhibit long serum half-lives, primarily due to the scavenging of antibodies from the endosomes by FcRn that recycles IgGs back out of cells.

In some particular examples, an engineered antibody as described herein displays greater pH-dependency in binding with FcRn than does endogenous IgG, in that antibodies as described herein display a greater absolute and/or relative differential change in affinity between serum pH and compartmental (e.g., endosomal) pH (or between serum and endosome) than does endogenous IgG. In some instances, an antibody having pH-dependent binding with FcRn displays greater binding to FcRn than do endogenous IgGs at a compartmental (e.g., endosomal) pH (e.g., at an acidic pH or at a pH equal to or less than pH 6.0). In some instances, an antibody having pH-dependent binding with FcRn displays greater binding to FcRn than do endogenous IgGs at a serum pH (e.g., in serum, at a neutral pH, or at a pH above pH 7.4).

In certain instances, an engineered antibody described herein displays pH-dependency, or enhanced pH dependency, in binding with FcRn and competes with endogenous IgG for interaction with FcRn. Accordingly, in some instances, an engineered antibody described herein binds FcRn at a level that is greater than endogenous IgG molecules (i.e., out-competes endogenous IgG molecules for binding with FcRn as a result of its greater affinity for FcRn) and/or results in a net increase in the rate of recycling of the FcRn-affinity-enhanced antibody relative to endogenous IgG molecules. In some instances, such preferential interaction of an engineered antibody described herein with FcRn, relative to endogenous IgG molecules, results in increased antibody half-life as compared to a reference antibody (e.g., a reference antibody comprising a heavy chain having the amino acid sequence of SEQ ID NO: 1 and a light chain having the amino acid sequence of SEQ ID NO: 2).

In some embodiments, an engineered antibody described herein exhibits a) increased affinity for FcRn at an acidic pH or compartmental pH, relative to that at serum pH and/or b) decreased affinity for C5 at an acidic pH or compartmental pH relative to that at serum pH. In some instances, such combination of features results in a synergistic increase in the processivity of the antibody that facilitates degradation of antigen, reduces buildup of antigen-antibody complex, and/or in cases where the antigen-antibody complex is cleared faster than the antibody itself it significantly increases half-life of antibody. This approach allows highly effective targeting and clearance of C5.

In some embodiments, where an engineered antibody has a higher affinity for C5 at serum pH than at compartmental pH (e.g., endosomal pH) and further includes an FcRn binding moiety that has a lower affinity for FcRn at serum pH than at compartmental pH (e.g., endosomal pH), such antibody binds serum C5 with high affinity to form an antibody/C5 complex. Upon internalization of the antibody/C5 complex by a cell (e.g., by pinocytosis) and into an internal compartment (e.g., an endosome) having a lower pH than serum pH, the lower affinity of antibody for C5 facilitates separation of antibody and C5, whereafter released C5 can be degraded by cellular machinery (e.g., by lysosomes). Further, increased affinity for FcRn at compartmental pH facilitates formation of antibody/FcRn complex, which complex can be recycled back into serum via the FcRn recycling pathway, within which the complex is exposed to serum or serum-like pH conditions, which pH facilitates release of antibody from the FcRn complex, e.g., back into serum. One net result of such processes can be a reduction the serum half-life of C5. Another net result of such processes can be an increase in serum half-life of antibody.

In various instances as disclosed herein, a serum pH can be, e.g., a pH or pH range typical or characteristic of serum, an individual or mean serum pH or pH range for one or more subjects, a standard pH value for serum, a measured pH value for serum, an estimated pH value for serum, or a selected pH value for serum. In various instances as disclosed herein, a serum pH can be a pH greater than 6.8, 6.9, 7.0, 7.1, 7.2, 7.3, 7.4, 7.5, 7.6, 7.7, 7.8, 7.9, 8.0, 8.1, 8.2, or greater. In various instances, a serum pH is a pH in the range of 6.8 to 8.2, 7.0 to 8.0, 7.0 to 7.8, 7.0 to 7.6, 7.0 to 7.4, or 7.0 to 7.2. In some embodiments, a serum pH is at or about pH 7.4

In various instances as disclosed herein, a compartmental pH can be, e.g., a pH or pH range typical or characteristic of an endosomal compartment (e.g., within an endosome), an individual or mean endosomal compartment pH or pH range for one or more subjects, a standard pH value for an endosomal compartment, a measured pH value for an endosomal compartment, an estimated pH value for an endosomal compartment, or a selected pH value for an endosomal compartment. In various instances as disclosed herein, a compartmental pH can be a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower. In various instances, a compartmental pH is a pH in the range of 5.0 to 7.2, 5.0 to 7.0, 5.0 to 6.8, 5.0 to 6.6, 5.0 to 6.4, 5.0 to 6.2, 5.0 to 6.0, 5.0 to 5.8, 5.0 to 5.6, 5.0 to 5.4, or 5.0 to 5.2.

In some embodiments, serum half-life of a serum C5 protein is decreased. For example, binding of an engineered antibody to serum C5 reduces serum half-life of C5 to fewer than about 7, 6, 5, 4, 3, 2, or 1 days, or less than about 24, 18, 12, or 6 hours.

In some embodiments, serum half-life of an engineered antibody is increased. For example, binding of an engineered antibody to FcRn increases serum half-life of the antibody to about 4 days to about 45 days, e.g., about 5 days to about 30 days, about 10 days to about 30 days, or about 20 days to about 30 days. In certain embodiments, an engineered antibody described herein has a serum half-life of about 5 days, about 10 days, about 15 days, about 20 days, about 25 days, about 30 days, about 35 days, about 40 days, about 45 days, about 50 days or longer.

In certain embodiments, an engineered antibody described herein exhibits a pH-dependent change in affinity for C5. Affinity may be measured as a K_{D}, equilibrium dissociation constant, of antibody and antigen; K_{D} and affinity are inversely related. In various embodiments, K_{D} of an engineered antibody as described herein for C5 at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for C5 at a serum pH is between 0.001 and 1 nM, e.g., 0.001 nM, 0.005 nM, 0.01 nM, 0.05 nM, 0.1 nM, 0.5 nM, or 1 nM. In some embodiments, K_{D} for C5 at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is higher than K_{D} of the same antibody for C5 at a serum pH or under serum conditions (and/or affinity of antibody for C5 at compartmental pH or under compartmental conditions may be decreased relative to affinity at a serum pH or under serum conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for C5 at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions may be, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody as described herein for C5 at a compartmental pH or under compartmental conditions may be, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more.

In some embodiments, an engineered antibody described herein exhibits a pH-dependent change in affinity for a receptor, such as an Fc receptor, e.g., FcRn. In various embodiments, K_{D} of an engineered antibody as described herein for FcRn at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions may be less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In some embodiments, K_{D} for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is higher than K_{D} of the same antibody for FcRn at a compartmental pH or under compartmental conditions (and/or affinity of antibody for FcRn at serum pH or under serum conditions may be decreased relative to affinity at a compartmental pH or under compartmental conditions), e.g., higher by at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is, e.g., greater than 10⁻¹⁵, 10⁻¹⁴, 10⁻¹³, 10⁻¹², 10¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody as described herein for FcRn at a serum pH or under serum conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more.

In some embodiments, an engineered antibody described herein exhibits both a pH-dependent change in affinity for C5 and a pH-dependent change an affinity for Fc receptor, e.g., FcRn. Thus, in some embodiments, an engineered antibody described herein exhibits greater affinity for C5 under serum conditions or at serum pH than under compartment conditions or at compartmental pH and also exhibits greater affinity for FcRn under compartmental conditions or at compartmental pH than under serum conditions or at serum pH. In various embodiments, K_{D} of an engineered antibody for C5 at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions may be less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an engineered antibody for C5 at a serum pH is between 0.001 and 1 nM, e.g., 0.001 nM, 0.005 nM, 0.01 nM, 0.05 nM, 0.1 nM, 0.5 nM, or 1 nM. In certain instances, K_{D} of an engineered antibody as described herein for C5 at a compartmental pH is between 0.001 and 1 nM, e.g., 0.001 nM, 0.005 nM, 0.01 nM, 0.05 nM, 0.1 nM, 0.5 nM, or 1 nM. In some embodiments, K_{D} for C5 at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is higher than K_{D} of the same antibody for C5 at a serum pH or under serum conditions (and/or affinity of antibody for C5 at compartmental pH or under compartmental conditions is decreased relative to affinity at a serum pH or under serum conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} for C5 at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody for C5 at a compartmental pH or under compartmental conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more. In some embodiments, K_{D} of an engineered antibody for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is higher than K_{D} of the same antibody for FcRn at a compartmental pH or under compartmental conditions (and/or affinity of antibody for FcRn at serum pH or under serum conditions is decreased relative to affinity at a compartmental pH or under compartmental conditions) by, e.g., at least 2-fold, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold 150-fold, 200-fold, 250-fold, 300-fold, 350-fold, 400-fold, 450-fold, 500-fold, 600-fold, 700-fold, 800-fold, 900-fold, 1000-fold, 2,000-fold, 3,000-fold, 4,000-fold, 5,000-fold, 6,000-fold, 7-000 fold, 8,000-fold, 9,000-fold, 10,000-fold, or more. In some embodiments, K_{D} of an engineered antibody for FcRn at a serum pH (e.g., a pH greater than 6.8, 6.9, 7.0, 7.1, 7.2, 7.3, 7.4, 7.5, 7.6, 7.7, 7.8, 7.9, 8.0, 8.1, 8.2, or greater) or under serum conditions is, e.g., greater than 10⁻¹⁵, 10⁻¹⁴, 10⁻¹³, 10⁻¹², 10⁻¹¹, 10⁻¹⁰, 10⁻⁹, 10⁻⁸, 10⁻⁷, 10⁻⁶, 10⁻⁵, 10⁻⁴, or 10⁻³ M. In certain instances, K_{D} of an engineered antibody for FcRn at a serum pH or under serum conditions is, e.g., equal to or greater than 1 nM, e.g., 1 nM, 2 nM, 3 nM, 4 nM, 5 nM, 10 nM, 20 nM, 30 nM, 40 nM, 50 nM, 100 nM, 200 nM, 300 nM, 400 nM, 500 nM, 600 nM, 700 nM, 800 nM, 900 nM, 1 mM, or more. In some embodiments, K_{D} of an engineered antibody for FcRn at a compartmental pH (e.g., a pH lower than 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, or lower) or under compartmental conditions is less than 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M.

In some embodiments, an engineered antibody described herein exhibits a greater half-life than a reference antibody (e.g., a reference antibody comprising a heavy chain having the amino acid sequence of SEQ ID NO: 1 and a light chain having the amino acid sequence of SEQ ID NO: 2) when administered to a subject, e.g., in the serum of the subject. In various instances, the half-life of a reference antibody (e.g., a reference antibody comprising a heavy chain having the amino acid sequence of SEQ ID NO: 1 and a light chain having the amino acid sequence of SEQ ID NO: 2) in serum may be, e.g., 250 to 300 hours. In various instances, the half-life in serum of an engineered antibody as described herein may be, e.g., at least 250 hours, e.g., at least 260, 270, 280, 290, or 300 hours. In certain embodiments, the half-life in serum of an engineered antibody as described herein may be at least 300 hours, e.g., at least 350, 400, 450, 500, 550, 600, 650, 700, 750, 800, 850, 900, 950, or 1,000 hours. In certain embodiments, the half-life in serum of an engineered antibody as described herein may be at least 1,000 hours, e.g., at least 1,500, 2,000, 2,500, 3,000, 3,500, 4,000, 4,500, 5,000, 6,000, 7,000, 8,000, 9,000, 10,000, 11,000, 12,000, 13,000, 14,000, or 15,000 hours or more. In various embodiments, the half-life in serum of an engineered antibody as described herein may be at least 12 days, 15 days, 20 days, 25 days, 30 days, 35 days, 40 days, 45 days, 50 days, 2 months, 3 months, 4 months, 5 months, 6 months, or more. In various instances, the half-life in serum of an engineered antibody as described herein may be increased as compared to a reference antibody (e.g., a reference antibody comprising a heavy chain having the amino acid sequence of SEQ ID NO: 1 and a light chain having the amino acid sequence of SEQ ID NO: 2) by a factor of at least, e.g., 2-fold, 3-fold, 4-fold, 5-fold, 6-fold, 7-fold, 8-fold, 9-fold, 10-fold, 20-fold, 30-fold, 40-fold, 50-fold, 75-fold, 100-fold or more.

In certain embodiments, an engineered antibody described herein exhibits an increased half-life in plasma, an increased mean retention time in plasma, and/or an increased level of C5 clearance (e.g., relative to a reference antibody, e.g., a reference antibody comprising a heavy chain having the amino acid sequence of SEQ ID NO: 1 and a light chain having the amino acid sequence of SEQ ID NO: 2). These parameters can be determined by methods known to those skilled in the art (e.g., as described in Nestorov et al., J. Clin. Pharmacol. 48:406-417 (2008); Leveque et al., Anticancer Research 25:2327-2344 (2005); Igawa et al., PLoS One 8: e63236. doi: 10.1371/journal.pone.0063236 (2013)). For example, an engineered antibody described herein (e.g., a single dose of such engineered antibody) reduces level of C5 in plasma by at least 10-fold, 50-fold, 100-fold, 250-fold, 500-fold, 750-fold, 1000-fold, 1500-fold, or more, relative to a reference antibody, e.g., a reference antibody comprising a heavy chain having the amino acid sequence of SEQ ID NO: 1 and a light chain having the amino acid sequence of SEQ ID NO: 2.

In some embodiments, an engineered antibody described herein inhibits cleavage of C5 (and/or levels of C5a and/or C5b in serum). In various embodiments, an engineered antibody as disclosed herein inhibits cleavage of C5 (and/or levels of C5a and/or C5b in serum) as compared to a prior measurement from the same patient or a reference value. In particular embodiments, administration of an engineered antibody as disclosed herein decreases the level or amount of C5 cleavage (and/or level or amount of C5a and/or level or amount of C5b) more than comparable administration of a reference antibody, e.g., a reference antibody comprising a heavy chain having the amino acid sequence of SEQ ID NO: 1 and a light chain having the amino acid sequence of SEQ ID NO: 2 (e.g., decreases level by more than about 10%, 20%, 30%, 40%, 50%, 60%, 70%, 80%, 90%, 100%, 150%, 2005, or more).

### Engineered Multi-specific Molecules

Methods and compositions described herein in the context of engineered anti-C5 antibodies can be applied to additional proteins to produce multi-specific binding molecules. Multi-specific binding molecules according to the present disclosure are engineered to include one or more binding moieties that specifically bind one or more targets of interest in a pH-dependent manner Multi-specific binding molecules encompass nucleic acids (e.g., RNA and DNA), proteins (e.g., antibodies), and combination thereof. pH-dependent binding moieties can be or include, for example, nucleic acids (e.g., RNA and DNA) and aptamers, polypeptides (e.g., antibodies or fragments thereof, albumin, receptors, ligands, signal peptides, avidin, and Protein A), polysaccharides, biotin, hydrophobic groups, hydrophilic groups, drugs, and any organic molecules that bind to receptors.

### Antibody or Fragment Thereof as Binding Moieties

In some embodiments, a multi-specific binding molecule described herein is an engineered antibody. In some instances, one or more binding moieties described herein are or include antibodies, antigen-binding fragments thereof, and/or Fc regions (or Fc fragments) thereof. The basic structure of an IgG antibody consists of two identical light polypeptide chains and two identical heavy polypeptide chains linked together by disulphide bonds. The first domain located at the amino terminus of each chain is variable in amino acid sequence, providing antibody binding specificities found in each individual antibody. These are known as variable heavy (VH) and variable light (VL) regions. The other domains of each chain are relatively invariant in amino acid sequence and are known as constant heavy (CH) and constant light (CL) regions. For an IgG antibody, the light chain includes one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

The term “Fc fragment”, as used herein, refers to one or more fragments of an Fc region that retains an Fc function and/or activity described herein, such as binding to an Fc receptor. The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). These antibody fragments can be obtained using conventional techniques known to those with skill in the art, and fragments can be screened for utility in the same manner as are intact antibodies.

Antibodies or fragments can be produced by any method known in the art for synthesizing antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

Additional antibodies of compositions and methods described herein are bispecific antibodies and multivalent antibodies, as described in, e.g., Segal et al., J. Immunol. Methods 248:1-6 (2001); and Tutt et al., J. Immunol. 147: 60 (1991).

In some embodiments, a multi-specific molecule described herein is an engineered antibody (e.g., engineered to have pH sensitive binding to antigen and to FcRn).

### Engineered Antigen Binding Regions of Engineered Multi-specific Molecules

In some embodiments, a binding moiety is or includes an antibody (e.g., an IgG antibody, e.g., an IgG1, IgG2, or IgG3 antibody), or an antigen binding fragment, engineered to bind to a target (i.e., antigen) in an altered manner (e.g., in a pH sensitive manner, e.g., in a more or less pH sensitive manner) relative to a reference antibody or antigen binding fragment. For example, an antibody can be engineered by modifying (e.g., by adding, deleting, or substituting) an amino acid within one or more antibody CDRs and/or at a position involved in antibody CDR structure. Exemplary, non-limiting sites of an antibody that can be modified include the following (amino acid positions are indicated based on the Kabat numbering (Kabat et al., (1991) Sequences of Proteins of Immunological Interest, NIH)).

Heavy chain: H27, H31, H32, H33, H35, H50, H58, H59, H61, H62, H63, H64, H65, H99, H100b, and H102

Light chain: L24, L27, L28, L32, L53, L54, L56, L90, L92, and L94.

In some embodiments, one or more of these disclosed amino acids can be substituted with histidine, arginine, lysine, aspartic acid, glutamic acid, serine, threonine, asparagine, or glutamine Without wishing to be bound by theory, it is believed that substituting an amino acid at one or more of these positions with a histidine can result in an antibody having pH-dependent antigen-binding properties. In some embodiments, a non-histidine residue is substituted with a histidine residue. In some embodiments, a histidine residue is substituted with a non-histidine residue. Additional engineered antigen binding regions include those described in, e.g., U.S. Publ. No. 20110229489.

### Engineered Constant Regions of Engineered Multi-Specific Molecules

In some instances, a binding moiety is or includes an antibody constant region, Fc region or Fc fragment that binds one or more Fc receptors (e.g., FcγRI, FcγRIIA, FcγRIIB, FcγRIIIA, FcγRIIIB, FcγRIV, or FcRn receptor). In some embodiments, a constant region, Fc region or Fc fragment is engineered to bind to a target (e.g., an Fc receptor) in an altered manner (e.g., in a pH sensitive manner, e.g., in a more or less pH sensitive manner) relative to a reference constant region, Fc region or Fc fragment.

In some instances, a binding moiety can be or include a constant region, Fc region or Fc fragment of an IgG antibody engineered to include an amino acid addition, deletion, or substitution, of one or more of amino acid residues described herein (e.g., 251-256, 285-290, 308-314, 385-389, and 428-436 (Kabat numbering (Kabat et al., (1991) Sequences of Proteins of Immunological Interest, NIH))).

### Producing Multi-specific Binding Molecules

In some embodiments, a multi-specific binding molecule described herein is engineered to include one or more binding moieties that exhibit pH sensitive binding to one or more targets by mutagenesis using known techniques. For example, a sequence of a reference polypeptide (e.g., a therapeutic antibody or therapeutic fusion protein) can be obtained, and one or more amino acid residues can be added, deleted, or substituted. In some embodiments, one or more amino acid residues are substituted with histidine, arginine, lysine, aspartic acid, glutamic acid, serine, threonine, asparagine, or glutamine In some embodiments, one or more amino acids are substituted with histidine. Without wishing to be bound by theory, it is believed that substitution of an amino acid residue with a histidine results in insertion of a protonation site, which can increase pH sensitivity of a binding moiety. Polypeptides can be produced using standard methods and assayed for binding to targets of interest as described herein. Additional methods of increasing pH sensitivity of a binding moiety are described in, e.g., Sarkar et al., Nature Biotechnology 20:908-913 (2002); Murtaugh et al., Protein Science 20:1619-1631 (2011); and U.S. Publ. No. 20110229489.

In some embodiments, a first target of interest is selected, and an antibody that selectively binds to the target is provided, obtained, and/or produced (e.g., using known methods as described herein). One or more amino acids of an antigen-binding region and/or an Fc region are substituted (e.g., with histidine, arginine, lysine, aspartic acid, glutamic acid, serine, threonine, asparagine, or glutamine), and pH sensitivity of binding to the target (and, additionally or alternatively, to FcRn) is determined. An antibody demonstrating desired binding affinity is selected as a multi-specific binding molecule.

In some embodiments, a polypeptide that naturally binds to a target of interest is provided, obtained, and/or produced. The polypeptide is conjugated to an Fc region or Fc fragment described herein (e.g., which binds to FcRn with a desired binding affinity) using known methods. For example, the polypeptide and Fc region or Fc fragment can be conjugated by chemical means or by recombinant expression as a fusion protein. Additionally or alternatively, one or more amino acids of the polypeptide can be substituted (e.g., with histidine, arginine, lysine, aspartic acid, glutamic acid, serine, threonine, asparagine, or glutamine), and pH sensitivity of binding of the polypeptide and the target is determined.

In some embodiments, a multi-specific binding molecule described herein is engineered to include one or more binding moieties identified and/or selected by screening. For example, an antigen-binding moiety that binds antigen in a pH sensitive manner can be identified using a library, e.g., a phage library, expressing antigen-binding moieties. Such a library can be screened for antigen-binding moieties that have a first affinity for antigen at a first pH (e.g., at pH 7.4) and that have a second affinity for antigen at a second pH (e.g., at pH 5.5). A multi-specific binding molecule described herein can be engineered to include such identified pH-sensitive antigen-binding moieties. Additionally and/or alternatively, an FcRn-binding moiety that binds FcRn in a pH sensitive manner can be identified using a library. Methods of screening recombinant antibody libraries are known (see, e.g., Hoogenboom, Nature Biotech. 23:1105-1116 (2005); U.S. Pat. No. 5,837,500; U.S. Pat. No. 5,571,698; WO 2012/044831).

### Targets

A multi-specific binding molecule described herein can bind to any target of interest. Exemplary, non-limiting targets include, e.g., complement C3 or C5, an IgG, an IgE, an IgM, CD20, CD40, IL1, IL2, IL4, IL5, IL6, IL8, IL10, IL12, IL21, IL22, IL23, Factor I, II, V, VII, VIII, IX, X, XI, XII, XIII, or their cognate receptors.

### Measuring Interactions of Binding Moieties and Targets

The binding properties of a multi-specific binding molecule described herein (e.g., an engineered antibody described herein) to a target (e.g., C5 and/or FcRn) can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and C5 and/or FcRn can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BlAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and _{Koff,} for the binding of a binding moiety to a target (e.g., an engineered antibody to C5 and/or FcRn). Such data can be used to compare different molecules. Information from SPR can also be used to develop structure-activity relationships (SAR). For example, the kinetic and equilibrium binding parameters of particular binding moieties to targets at various pH levels can be evaluated. Variant amino acids at given positions can be identified that correlate with particular binding parameters, e.g., high affinity, low affinity, and slow K_{off}, at particular pH levels.

### Reference Proteins

In some embodiments, methods of the disclosure include modification of a reference protein, e.g., a reference antibody, e.g., a reference therapeutic antibody, and/or comparison of one or more properties of a multi-specific binding molecule (e.g., engineered antibody described herein) to a reference protein. Any known protein, e.g., antibody, can be a reference protein or antibody in a method described herein. Exemplary, non-limiting reference proteins include abciximab (ReoPro®, Roche), adalimumab (Humira®, Bristol-Myers Squibb), alemtuzumab (Campath®, Genzyme/Bayer), basiliximab (Simulect®, Novartis), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denosumab (Prolia®, Amgen; Xgeva®, Amgen), eculizumab (Saris®, Alexion Pharmaceuticals), efalizumab (Raptiva®, Genentech), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and ustekinumab (Stelara®, Janssen). In some embodiments, a reference protein is a foregoing protein having 1, 2, 3, 4, 5, 6, 7, 8, 9, 10, 11, 12, or more amino acid substitutions in its amino acid sequence, and/or a protein having 85%, 86%, 87%, 88%, 89%, 90%, 91%, 92%, 93%, 94%, 95%, 96%, 97%, 98%, 99%, or 100% identity to the amino acid sequence of a foregoing protein.

### Methods of Treatment

In some embodiments, a multi-specific binding molecule described herein (e.g., an engineered antibody as described herein) is used in a method of treating one or more complement-associated conditions. In some embodiments, a multi-specific binding molecule described herein (e.g. an engineered antibody as described herein) is for use as a medicament. Complement-associated conditions can include, without limitation, conditions that are caused by, include, include symptoms resulting in whole or in part from, or are known to occur in conjunction with increased or decreased complement activity. C5 is a key to pathology and amenable to C5 blockade in hematologic, neurologic and renal disorders among others. Examples of complement-associated conditions include, without limitation, peripheral neuropathy, cryoglobulinemia, cryoglobulinemic neuropathy, neurosarcoidosis age-related macular degeneration (AMD), Alzheimer's disease, amyotrophic lateral sclerosis (ALS), antiphospholipid syndrome (or antiphospholipid antibody syndrome or Hughes syndrome),vasculitic neuropathy, Reflex Sympathetic Dystrophy, Complex Regional Pain Syndrome, Chronic Inflammatory Demyelinating Polyneuropathyantineutrophil cytoplasm antibody (ANCA)-associated vasculitis (AAV), asthma, atherosclerosis, atypical hemolytic-uremic syndrome (aHUS), autoimmune hemolytic anemia, brain injury, C3 nephropathy, capillary leak syndrome, cardiopulmonary bypass and hemodialysis, cardiovascular disorders, catastrophic antiphospholipid syndrome, cerebrovascular disorders, chronic inflammatory demyelinating neuropathy, cold agglutinin disease (CAD), Degos disease, dense deposit disease (DDD), dermatitis, inflammatory myopathies, dermatomyositis, myositis, antibody-induced myositis, diabetic angiopathy, diabetic retinopathy, dilated cardiomyopathy, Disseminated Intravascular Coagulation (DIC), elevated liver enzymes, epidermolysis bullosa, erythematosus-associated vasculitis, glomerulonephritis, Goodpastures syndrome, Graves' disease, Guillain-Barre syndrome (GBS), Hashimoto's thyroiditis, HELLP syndrome, hemolysis, Sickle Cell Disease Henoch-Schonlein purpura nephritis, idiopathic thrombocytopenic purpura (ITP), injury resulting from myocardial infarction, ischemia-reperfusion injury, Kawasaki's disease, lupus nephritis, immune complex vasculitis, macular degeneration (e.g., age-related macular degeneration (AMD), mesenteric/enteric vascular disorders, multifocal motor neuropathy, multiple sclerosis, myasthenia gravis, myocarditis, Neonatal Allo-Immune Thrombocytopenia (NAITP), neuromyelitis optica, organ or tissue transplantation, paroxysmal cold hemoglobinuria, paroxysmal nocturnal hemoglobinuria (PNH), Pauci-immune vasculitis, pemphigus, percutaneous transluminal coronary angioplasty, peripheral vascular disorders, Post-Transfusion Purpura, psoriasis, recurrent fetal loss, renovascular disorders, restenosis following stent placement, revascularization to transplants and/or replants, rotational atherectomy, rheumatoid arthritis, psoriatic arthritis, scleroderma, sepsis, septic shock, shiga toxin E. coli-related hemolytic uremic syndrome (STEC-HUS), spinal cord injury, spontaneous fetal loss, systemic inflammatory response, glomerulonephritis, systemic lupus, systemic lupus erythematosus (SLE), systemic lupus erythematosus-associated vasculitis, Takayasu's disease, thoracic-abdominal aortic aneurysm, thrombotic thrombocytopenic purpura (TTP), transplant rejection, traumatic brain injury, type I diabetes, typical or infectious hemolytic uremic syndrome, vasculitis, vasculitis associated with rheumatoid arthritis, and venous gas embolus, or any complement-associated inflammatory response.

Complement-associated disorders include complement-associated pulmonary disorders such as, but not limited to, asthma, bronchitis, a chronic obstructive pulmonary disease (COPD), an interstitial lung disease, al anti-trypsin deficiency, emphysema, bronchiectasis, bronchiolitis obliterans, alveolitis, sarcoidosis, pulmonary fibrosis, and collagen vascular disorders.

In some embodiments, a multi-specific binding molecule described herein (e.g., an engineered antibody described herein) can be used to treat graft rejection/graft-versus-host disease (GVHD), reperfusion injuries (e.g., following cardiopulmonary bypass or a tissue transplant), and tissue damage following other forms of traumatic injury such as a burn (e.g., a severe burn), blunt trauma, spinal injury, or frostbite. In some embodiments, a multi-specific binding molecule described herein (e.g., an engineered antibody described herein), alone or in combination with a second anti-inflammatory agent, can be used to treat an inflammatory disorder such as, but not limited to, RA (above), inflammatory bowel disease, sepsis (above), septic shock, acute lung injury, disseminated intravascular coagulation (DIC), or Crohn's disease. In some embodiments, the second anti-inflammatory agent can be one selected from the group consisting of NSAIDs, corticosteroids, methotrexate, hydroxychloroquine, anti-TNF agents such as etanercept and infliximab, a B cell depleting agent such as rituximab, an interleukin-1 antagonist, or a T cell costimulatory blocking agent such as abatacept. In some embodiments, a multi-specific binding molecule described herein (e.g., an engineered antibody described herein) is useful for treating allergic asthma, allergic rhinitis, hyper-IgE syndrome/Job's syndrome, food allergies, Paroxysmal nocturnal hemoglobinurea (PNH), inflammatory bowel disease and/or other large-organ cytokine-mediated inflammatory conditions.

In some embodiments, an engineered antibody as described herein is used in a method of treating PNH or aHUS. Paroxysmal nocturnal hemoglobinuria (PNH), previously Marchiafava—Micheli syndrome, is a rare, acquired, life-threatening disease of the blood characterized by destruction of red blood cells by the complement system.

In various instances, a multi-specific binding molecule described herein (e.g., an engineered antibody described herein) treats, alleviates, reduces the prevalence of, reduces the frequency of, or reduces the level or amount of one or more symptoms or biomarkers of PNH. Symptoms and biomarkers of PNH include, without limitation, hemolysis, abdominal pain (e.g., severe abdominal pain or stomach pain), leg pain, leg swelling, headaches (e.g., severe headaches), back pain, weakness, fatigue (e.g., tiredness, difficulty performing daily activities, trouble concentrating, dizziness, weakness), shortness of breath, difficulty swallowing, yellowing of the skin, yellowing of the eyes, erectile dysfunction, anemia, pulmonary hypertension, recurrent infection, susceptibility to infection, colored urine (e.g., dark color), Budd-Chiari syndrome, heart palpitations, myelodysplasia, acute leukemia, menorrhagia, lightheadedness, irritability, red blood in urine, thrombosis (e.g., in veins, e.g., hepatic vein thrombosis or sagittal vein thrombosis), smooth muscle dystonias, abdominal contractions, esophageal spasms, chronic renal disease, Ham's acid hemolysis test results, sucrose hemolysis test results, binding of monoclonal antibodies directed against cell-bound complement regulators (CD59, CD24, CD66b, CD16, fluorescently-labeled aerolysin (FLAER)) to a peripheral blood sample, high serum lactate dehydrogenase, serum creatinine levels, fibrinolysis, plasmin-mediated clot degradation, D-dimer levels, and others known in the art. Symptoms may be increased following any of infection, alcohol, exercise, or stress. Specific symptoms and progression of symptoms vary among subjects.

Thus, in some embodiments, a multi-specific binding molecule described herein (e.g., an engineered antibody described herein) is administered to a subject in need thereof, e.g., a subject having Paroxysmal Nocturnal Hemoglobinuria (PNH) or a subject having Atypical Hemolytic Uremic Syndrome (aHUS).

In various instances, administration of a multi-specific binding molecule described herein (e.g., an engineered antibody described herein) results in a decrease in the prevalence, frequency, level, and/or amount of one or more symptoms or biomarkers of PNH as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

In some embodiments, administration of a multi-specific binding molecule described herein (e.g., an engineered antibody described herein) to a subject having PNH results in a greater decrease or improvement in one or more symptoms or biomarkers of PNH than does a reference antibody, e.g., a reference antibody comprising a heavy chain having the amino acid sequence of SEQ ID NO: 1 and a light chain having the amino acid sequence of SEQ ID NO: 2, under comparable conditions. Treatment of subjects having PNH according can result in decreased frequency or likelihood of increased symptoms following any of infection, alcohol, exercise, or stress.

Atypical hemolytic-uremic syndrome (aHUS) is a disease that primarily affects kidney function. This condition, which can occur at any age, can cause abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure. Mutations in the genes associated with atypical hemolytic-uremic syndrome lead to uncontrolled activation of the complement system. The overactive system attacks cells that line blood vessels in the kidneys, causing inflammation and the formation of abnormal clots. These abnormalities lead to kidney damage and, in many cases, kidney failure and ESRD.

In various instances, a multi-specific binding molecule described herein (e.g., an engineered antibody described herein) treats, alleviates, reduces the prevalence of, reduces the frequency of, or reduces the level or amount of one or more symptoms or biomarkers of aHUS. Symptoms of aHUS include, without limitation, nausea, vomiting, confusion, shortness of breath (dyspnea), fatigue, anemia, thrombocytopenia, kidney damage, kidney failure, end-stage renal disease, stroke, gastrointestinal issues (e.g., severe stomach pain), colon inflammation, blood vessel damage, heart attacks, neurological issues (e.g., seizures), anemia, hemolysis, pale skin, jaundice, edema, rapid heart rate, yellowing of the eyes, thrombotic microangiopathy (TMA), transplant-associated thrombotic microangiopathy (TA-TMA), stroke, heart attack, malaise, microangiopathic anemia, bloody diarrhea, lung complications, pancreatitis, schistocytes, encephalopathy, coma, malignant hypertension, proteinuria, decreased platelets, decreased hemoglobulin, decreased heptaglobin, increased lactate dehydrogenase (LDH), increased creatine, and/or increased blood urea nitrogen.

In various instances, administration of a multi-specific binding molecule described herein (e.g., an antibody described herein) to a subject having aHUS results in a decrease in the prevalence, frequency, level, or amount of one or more symptoms or biomarkers of aHUS as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having aHUS results in a greater decrease or improvement in one or more symptoms of aHUS than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating acquired microthrombotic diseases. Acquired microthrombotic diseases characterized by thrombocytopenia, hemolytic anemia, renal failure affect 4 per million globally and 20% of hematopoietic stem cell recipients in the US (8000 per year in US). Thrombotic microangiopathy, abbreviated TMA, is a pathology that results in thrombosis in capillaries and arterioles, due to an endothelial injury. These diseases can be a result of genetic complement mutations such as aHUS. They can also be secondary to some medications, stem cell transplant, infection-related, pregnancy, surgery, malignancy, or STEC-associated. It may be seen in association with thrombocytopenia, anemia, purpura and renal failure. The classic TMAs are hemolytic uremic syndrome and thrombotic thrombocytopenic purpura. Other conditions with TMA include atypical hemolytic uremic syndrome, disseminated intravascular coagulation, scleroderma renal crisis, malignant hypertension, antiphospholipid antibody syndrome, and drug toxicities, e.g. calcineurin inhibitor toxicity. TMAs often result in decreased endothelial thromboresistance, leukocyte adhesion to damaged endothelium, complement consumption, enhanced vascular shear stress, and abnormal von Williebrand factor (vWF) fragmentation.

Transplant-associated thrombotic microangiopathy (TA-TMA) can exhibit complications of the transplant itself, including infection, graft-versus-host disease, and disseminated intravascular coagulation, as well as the side effects of immunosuppressive drugs, can mimic a TMA. Because the pathophysiology of TA-TMA is poorly understood, current treatment options are suboptimal, and the condition carries a very high mortality rate. In various instances, a multi-specific binding molecule described herein (e.g., an engineered antibody described herein) treats, alleviates, reduces the prevalence of, reduces the frequency of, or reduces the level or amount of one or more symptoms or biomarkers of TMA or TA-TMA. Symptoms of TA-TMA include, without limitation, fever, microangiopathic hemolytic anemia (see schistocytes in a blood smear), renal failure, thrombocytopenia, neurological manifestations, multi-organ failure or injury is also possible, affecting the brain, kidneys, heart, liver, and other major organs.

The vast majority of TA-TMA patients lack suppression of ADAMTS13 (a disintegrin and metalloproteinase with a thrombospondin type 1 motif, member 13) activity to less than 5% to 10% of normal and do not have a complete response to plasma exchange. In addition, the presentation of TA-TMA is highly heterogeneous, ranging from asymptomatic, low-level red blood cell fragmentation to fulminant disease. The diagnosis of TA-TMA is made most reliably by examination of the peripheral blood film for red blood cell fragments.

In various instances, administration of a multi-specific binding molecule described herein (e.g., an antibody described herein) to a subject having TA-TMA results in a decrease in the prevalence, frequency, level, or amount of one or more symptoms or biomarkers of TA-TMA as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having TA-TMA results in a greater decrease or improvement in one or more symptoms of TA-TMA than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating C3 Glomerulopathies (C3G). C3 Glomerulopathies (C3G) are a group of rare renal diseases characterized by C3 deposition without immunoglobulin deposits. These renal diseases can be subdivided into Dense Deposit Disease (DDD) and C3 glomerulonephritis (C3GN) based on histology. C3G patients have in common mutations in alternate complement pathway genes, the presence of C3 nephritic factors and a substantial risk for both ESRD and recurrence of disease after renal transplant . C3 convertase autoantibodies stabilize the C3 convertase complex and increase local generation of the alternative pathway complement. Kidney failure occurs to 50% of patients within 10 years with limited renal transplant in C3 diseases by 50% recurrence and graft loss post-transplant.

Dense deposit disease (DDD), a very rare kidney disease characterized on a renal biopsy by an abundance C3 in the renal glomeruli, and named for the extremely dense deposits seen in the glomerular basement membrane (GBM) using electron microscopy. In both DDD and C3GN, deposits of C3 and other proteins in the GBM disrupt kidney function.

Progressive damage to the glomeruli occurs eventually resulting in kidney failure. When kidney failure occurs, dialysis must be started or transplantation must be performed. The rate of progression to end-stage kidney failure and dialysis appears to be similar for both DDD and C3GN. In addition to dense deposits in the kidney, persons with DDD can develop deposits in the eyes. The signs and symptoms of DDD and C3GN are similar, including but not limited to hematuria, proteinuria, white blood cells in the urine; edema, high blood pressure, decreased urine output; and decreased alertness.

If C3G (either DDD or C3GN) is suspected, immunofluorescence analysis should show abundant C3 in the glomerular capillaries. In addition to C3, the glomeruli may contain other complement system proteins with reduced levels of complement proteins in blood circulation. The causes of complement dysregulation can be genetic variants in complement regulatory proteins and autoantibodies to the convertases [5, 9, 11, 12, 13, 14, 16, 17, 18, 19, 20, 21, 22, 23, 24, 25, 26, 27, 28, 29]; mutations in complement Factor H (CFH), complement Factor I (CFI), MCP (also termed membrane cofactor protein or CD46), complement Factor B (CFB), complement Factor C3 and CFHRS. Complement dysregulation may also be due to acquired factors.

In various instances, administration of a multi-specific binding molecule described herein (e.g., an antibody described herein) to a subject having DDD or C3GN results in a decrease in the prevalence, frequency, level, or amount of one or more symptoms or biomarkers of DDD or C3GN as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having DDD or C3GN results in a greater decrease or improvement in one or more symptoms of DDD or C3GN than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, an engineered antibody as described herein is used in a method of treating multiple neuropathy. Complement activation in peripheral neurons during injury results in multiple neuropathy subtypes such as hereditary/familial amyloid neuropathy; Guillain- Barré syndrome (GBS), a demyelinating neuropathy can damage motor, sensory, and autonomic nerve fibers; Diabetic neuropathy; etc. Peripheral neuropathy (PN) can be caused by inflammation of, or damage to, the nerves. It can result in tingling, numbness and burning pain in any part of the body, but commonly is felt in the hands, feet and lower legs. Some patients may experience an increased sensitivity to pain, loss of sensitivity to temperature, sensorimotor impairment due to nerve damage.

The systemic amyloidoses are a diverse group of disorders that can lead to multi-organ dysfunction through the deposition of abnormal amyloid fibrils. Hereditary amyloid peripheral neuropathies can be further classified according to the type of amyloid protein that causes the disease process. These include transthyretin, apoprotein Al, gelsolin, and Aβ2-microglobulin. Mutations in the TTR gene lead to the most common form of inherited amyloidosis, whereas amyloid light chain (AL) amyloidosis is the most common acquired form. Peripheral nervous system involvement is common and may present as a length dependent sensorimotor polyneuropathy, focal neuropathy, multi-focal neuropathy, or autonomic neuropathy. Familial Amyloid Polyneuropathy (FAP) refers to a group of hereditary amyloidoses which typically have prominent clinical manifestations involving the peripheral sensorimotor and/or autonomic nervous system.

Guillain-Barré syndrome (GBS) is a rapid-onset muscle weakness caused by the immune system damaging the peripheral nervous system. The initial symptoms are typically changes in sensation or pain along with muscle weakness, beginning in the feet and hands spreads to the arms and upper body with both sides being involved.. Symptoms of Guillain-Barre syndrome often begins with tingling and weakness starting in the feet and legs, spreading to the upper body and arms, difficulty with eye or facial movements, including speaking, chewing or swallowing, severe pain, difficulty with bladder control or bowel function, rapid heart rate, low or high blood pressure and difficulty breathing. As GBS progresses, muscle weakness can evolve into paralysis. Guillain-Barre syndrome is now known to occur in several forms. The main types are Acute inflammatory demyelinating polyradiculoneuropathy (AIDP), Miller Fisher syndrome (MFS), Acute motor axonal neuropathy (AMAN) and acute motor-sensory axonal neuropathy (AMSAN).

Diabetic neuropathies are a family of nerve disorders caused by diabetes. People with diabetes can, over time, develop nerve damage throughout the body. Some people with nerve damage have no symptoms. Others may have symptoms such as pain, tingling, or numbness, loss of feeling in the hands, arms, feet, and legs. Nerve problems can occur in every organ system, including the digestive tract, heart, and sex organs. Symptoms of nerve damage may include numbness, tingling, or pain in the toes, feet, legs, hands, arms, and fingers, muscle wasting, indigestion, nausea, or vomiting diarrhea or constipation, dizziness or faintness due to a drop in blood pressure after standing or sitting up, problems with urination, erectile dysfunction in men or vaginal dryness in women.

In various instances, administration of a multi-specific binding molecule described herein (e.g., an antibody described herein) to a subject having a peripheral neuropathy results in a decrease in the prevalence, frequency, level, or amount of one or more symptoms or biomarkers of a peripheral neuropathy as described herein or otherwise known in the art, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms as compared to a prior measurement in the subject or to a reference value.

In some embodiments, administration of a multi-specific binding molecule described herein (e.g., an engineered antibody described herein) to a subject having a peripheral neuropathy results in a greater decrease or improvement in one or more symptoms of a peripheral neuropathy than does a reference protein, e.g., a reference antibody comprising a heavy chain having the amino acid sequence of SEQ ID NO: 1 and a light chain having the amino acid sequence of SEQ ID NO: 2, under comparable conditions.

In some embodiments, a multi-specific binding molecule described herein (e.g., an engineered antibody as described herein) exhibits a decreased effective dose as compared to a reference protein (e.g., a reference antibody comprising a heavy chain having the amino acid sequence of SEQ ID NO: 1 and a light chain having the amino acid sequence of SEQ ID NO: 2). For instance, an effective dose of an engineered antibody as described herein may be, e.g., less than 1,000 mg/dose, e.g., less than 900 mg/dose, 800 mg/dose, 700 mg/dose, 600 mg/dose, 500 mg/dose, 550 mg/dose, 400 mg/dose, 350 mg/dose, 300 mg/dose, 200 mg/dose, 100 mg/dose, 50 mg/dose, 25 mg/dose, or less. In certain instances, an effective dose of an engineered antibody as disclosed herein is lower than an effective or recommended or approved dosage of a reference antibody, e.g., a reference antibody comprising a heavy chain having the amino acid sequence of SEQ ID NO: 1 and a light chain having the amino acid sequence of SEQ ID NO: 2, which dosage of a reference antibody may be, e.g., 900 mg/dose or 600 mg/dose. Alternatively or in combination with a dosage as disclosed herein, an engineered antibody as described herein may be effectively or usefully administered at a frequency that is less than once per week, e.g., less than once every week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, or year. In certain instances, an effective or useful administration frequency of an engineered antibody as disclosed herein is lower than an effective or recommended or approved administration frequency of a reference antibody, e.g., a reference antibody comprising a heavy chain having the amino acid sequence of SEQ ID NO: 1 and a light chain having the amino acid sequence of SEQ ID NO: 2, which administration frequency can be administered weekly (e.g., at a dosage of 300-600 mg, depending on weight of subject) or every two weeks (e.g., at a dosage of 300-1200 mg, depending on weight of subject).

In some embodiments, a multi-specific binding molecule described herein (e.g., an engineered antibody described herein) can be administered at a decreased dose amount as compared to a reference protein, e.g., a reference antibody comprising a heavy chain having the amino acid sequence of SEQ ID NO: 1 and a light chain having the amino acid sequence of SEQ ID NO: 2, while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2). In some embodiments, an engineered antibody described herein can be administered at an increased interval as compared to a reference antibody (e.g., a reference antibody comprising a heavy chain having the amino acid sequence of SEQ ID NO: 1 and a light chain having the amino acid sequence of SEQ ID NO: 2) while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2). In some embodiments, an engineered antibody described herein can be administered in a decreased number of unit dosages, and/or for a decreased period of treatment, as compared to a reference antibody (e.g., a reference antibody comprising a heavy chain having the amino acid sequence of SEQ ID NO: 1 and a light chain having the amino acid sequence of SEQ ID NO: 2) while achieving an equal, equally effective, comparably effective, or substantially effective outcome, where the engineered antibody is administered in an identical, equivalent, or substantially equivalent formulation and/or by an identical, equivalent, or substantially equivalent route of administration as the reference (e.g., a reference antibody comprising a heavy chain having the amino acid sequence of SEQ ID NO: 1 and a light chain having the amino acid sequence of SEQ ID NO: 2).

In accordance with some such embodiments, an administered dose of an engineered antibody described herein may be less likely to elicit an adverse response when administered to a subject, e.g., an adverse immune response, than would an effective dose of a reference antibody, e.g., a reference antibody comprising a heavy chain having the amino acid sequence of SEQ ID NO: 1 and a light chain having the amino acid sequence of SEQ ID NO: 2. Accordingly, in various embodiments, an engineered antibody as disclosed herein may be less likely than a reference antibody, e.g., a reference antibody comprising a heavy chain having the amino acid sequence of SEQ ID NO: 1 and a light chain having the amino acid sequence of SEQ ID NO: 2, per unit of activity administered to induce an adverse reaction or side effect. In various embodiments, an engineered antibody as disclosed herein may less likely than a reference antibody, e.g., a reference antibody comprising a heavy chain having the amino acid sequence of SEQ ID NO: 1 and a light chain having the amino acid sequence of SEQ ID NO: 2, per unit of activity administered, to induce an adverse reaction or side effect having a particular degree of severity. In various embodiments, an engineered antibody as disclosed herein may induce one or more adverse reactions or side effects to a lesser degree or in fewer patients than a reference antibody, e.g., a reference antibody comprising a heavy chain having the amino acid sequence of SEQ ID NO: 1 and a light chain having the amino acid sequence of SEQ ID NO: 2, per unit of activity administered. Examples of adverse reactions or side effects that may be associated with the administration of an antibody capable of binding C5, e.g., a prior antibody such as a reference antibody comprising a heavy chain having the amino acid sequence of SEQ ID NO: 1 and a light chain having the amino acid sequence of SEQ ID NO: 2, can include headache, nasopharyngitis, back pain, nausea, diarrhea, hypertension, upper respiratory infection, abdominal pain, vomiting, anemia, cough, peripheral edema, and/or urinary tract infection.

In some embodiments, administration of a multi-specific binding molecule described herein (e.g., an engineered antibody described herein) decreases C5 titer in serum. A typical concentration of C5 in human plasma is about 0.37 μM. Upon administration of one or more doses of an engineered antibody as disclosed herein, the concentration of human C5 in plasma may be reduced in a subject as compared to a prior measured concentration in the same subject or as compared to a standard value, e.g., as compared to a value of about 0.37 μM. In various instances, a concentration of C5 in serum following administration of an engineered antibody, including any number of doses (e.g., 1 dose, 3 doses, or a number of doses prescribed over a period of months or years) administered over any period of time (e.g., 1-4 weeks, 1-12 months, or 1-3 or more years) in any of one or more subjects or an aggregate of subjects, may be equal to or less than, e.g., 0.35 μM, 0.325 μM, 0.30 μM, 0.275 μM, 0.25 μM, 0.225 μM, 0.20 μM, 0.175 μM, 0.15 μM, 0.125 μM, 0.10 μM, 0.075 μM, 0.05 μM, or 0.025 μM. In some embodiments, upon administration to a subject, a multi-specific binding molecule described herein (e.g., an engineered antibody described herein) results in a greater decrease in C5 titer in serum that does a reference protein, e.g., a reference antibody comprising a heavy chain having the amino acid sequence of SEQ ID NO: 1 and a light chain having the amino acid sequence of SEQ ID NO: 2 a reference antibody comprising a heavy chain having the amino acid sequence of SEQ ID NO: 1 and a light chain having the amino acid sequence of SEQ ID NO: 2, under comparable conditions.

In some embodiments, upon administration to a subject (e.g., at a single dose), a multi-specific binding molecule described herein (e.g., an engineered antibody described herein) is measured at an increased level in plasma at a defined time following administration (e.g., 1, 2, 3, 4, 5, 6, 7, 8, 9, 10, 11, 12, 13, 14, or more days), relative to level of a control at the same defined time (e.g., a reference protein, e.g., a reference antibody comprising a heavy chain having the amino acid sequence of SEQ ID NO: 1 and a light chain having the amino acid sequence of SEQ ID NO: 2). For example, at a defined time following administration of a single dose, a level of an engineered antibody described herein is at least 10%, 20%, 30%, 40%, 50%, 60%, 70%, 80%, 90%, 100%, 150%, 200%, 300%, 400%, or 500% higher than a corresponding level of a reference antibody, e.g., a reference antibody comprising a heavy chain having the amino acid sequence of SEQ ID NO: 1 and a light chain having the amino acid sequence of SEQ ID NO: 2.

In some embodiments, a multi-specific binding molecule described herein (e.g., an engineered antibody described herein) is measured at an increased level in plasma at a defined time (e.g., 1, 2, 3, 4, 5, 6, 7, 8, 9, 10, 11, 12, 13, 14, or more days) following administration (e.g., of a single dose), relative to level of a control at the same defined time (e.g., a reference protein, e.g., a reference antibody comprising a heavy chain having the amino acid sequence of SEQ ID NO: 1 and a light chain having the amino acid sequence of SEQ ID NO: 2). For example, at a defined time following administration, a level of an engineered antibody described herein is at least 10%, 20%, 30%, 40%, 50%, 60%, 70%, 80%, 90%, 100%, 150%, 200%, 300%, 400%, or 500% higher than a corresponding level of a reference antibody (e.g., a reference antibody comprising a heavy chain having the amino acid sequence of SEQ ID NO: 1 and a light chain having the amino acid sequence of SEQ ID NO: 2).

In some embodiments, an engineered antibody described herein has increased half-life (e.g., relative to a control, e.g., a reference antibody, e.g., a reference antibody comprising a heavy chain having the amino acid sequence of SEQ ID NO: 1 and a light chain having the amino acid sequence of SEQ ID NO: 2), and thus the engineered antibody can be administered to a subject at increased inter-dose intervals. For example, an engineered antibody can be administered once every week, every two weeks, every three weeks, every four weeks, every 6 weeks, every 8 weeks, or longer duration.

In some embodiments, a therapeutically effective amount of a multi-specific binding molecule described herein (e.g., an engineered antibody described herein) is about 90%, 80%, 70%, 60%, 50%, 40%, 30%, 20%, 10%, or 5% of an effective amount of a reference therapeutic protein, e.g., a reference antibody comprising a heavy chain having the amino acid sequence of SEQ ID NO: 1 and a light chain having the amino acid sequence of SEQ ID NO: 2. In some embodiments, a single dose of an engineered antibody described herein achieves a comparable therapeutic effect as two or more doses of a reference antibody, e.g., a reference antibody comprising a heavy chain having the amino acid sequence of SEQ ID NO: 1 and a light chain having the amino acid sequence of SEQ ID NO: 2.

In some embodiments, a multi-specific binding molecule described herein (e.g., an engineered antibody described herein) is administered at a dose that is about 90%, 80%, 70%, 60%, 50%, 40%, 30%, 20%, 10%, or 5% of the concentration of a target antigen (e.g., C5) in the serum of the subject.

In some embodiments, a multi-specific binding molecule described herein (e.g., an engineered antibody described herein) can be administered by a route other than intravenous administration, e.g., by subcutaneous administration. Thus, in various embodiments, an antibody as disclosed herein can be administered subcutaneously. In some embodiments, an engineered antibody described herein can be administered by intravenous and subcutaneous routes, e.g., as components of single treatment strategy. Intravenous and subcutaneous administration may be concurrent or non-concurrent.

In some embodiments, a multi-specific binding molecule described herein (e.g., an engineered antibody described herein) can be used in a number of diagnostic and therapeutic applications. For example, detectably-labeled versions of engineered antibodies as described herein can be used in assays to detect the presence or amount of the C5 in a sample (e.g., a biological sample). Engineered antibodies described herein can be used in in vitro assays for studying inhibition of C5 activity and/or cleavage. In some embodiments, an engineered antibody described herein can be used as a positive control in an assay designed to identify additional novel compounds that inhibit complement activity or otherwise are useful for treating a complement-associated disorder. For example, an engineered antibody described herein can be used as a positive control in an assay to identify additional compounds (e.g., small molecules, aptamers, or antibodies) that reduce or abrogate C5 production or formation of MAC.

Multi-specific binding molecules described herein (e.g., engineered antibodies as described herein) may be used in monitoring a subject, e.g., a subject having, suspected of having, at risk of developing, or under treatment for one or more complement-associated conditions. Monitoring may include determining the amount or activity of C5 in a subject, e.g., in the serum of a subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration of an engineered antibody as described herein. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a complement-associated disorder described herein.

### Formulations and Administration

In various embodiments, a multi-specific binding molecule described herein (e.g., an engineered antibody described herein) can be incorporated into a pharmaceutical composition. Such a pharmaceutical composition can be useful, e.g., for the prevention and/or treatment of diseases, e.g., PNH and/or aHUS, or other complement-associated disorder. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

A suitable means of administration can be selected based on the age and condition of a subject. A single dose of the pharmaceutical composition containing a multi-specific binding molecule described herein (e.g., an engineered antibody described herein)can be selected from a range of 0.001 to 1000 mg/kg of body weight. On the other hand, a dose can be selected in the range of 0.001 to 100000 mg/body weight, but the present disclosure is not limited to such ranges. The dose and method of administration varies depending on the weight, age, condition, and the like of the patient, and can be suitably selected as needed by those skilled in the art.

In various instances, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

In particular instances, a pharmaceutical composition can be formulated as a solution. In some embodiments, a composition can be formulated, for example, as a buffered solution at a suitable concentration and suitable for storage at 2-8° C. (e.g., 4° C.).

Compositions including one or more engineered antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, compositions can be formulated in a composition suitable for intrapulmonary administration (e.g., for administration via an inhaler or nebulizer) to a mammal such as a human. Methods for formulating such compositions are well known in the art. Dry powder inhaler formulations and suitable systems for administration of the formulations are also known in the art. Pulmonary administration may be oral and/or nasal. Examples of pharmaceutical devices for pulmonary delivery include metered dose inhalers, dry powder inhalers (DPIs), and nebulizers. For example, a composition described herein can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TURBOHALER® (AstraZeneca; London, England) the AIR® inhaler (ALKERMES®; Cambridge, Mass.); ROTAHALER® (GlaxoSmithKline; London, England); and ECLIPSE™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. In some embodiments, a composition described herein can be intrapulmonarily administered by way of a metered dose inhaler. These inhalers rely on a propellant to deliver a discrete dose of a compound to the lungs. Additional devices and intrapulmonary administration methods are set forth in, e.g., U.S. Patent Application Publication Nos. 20050271660 and 20090110679, the disclosures of each of which are incorporated herein by reference in their entirety.

In some embodiments, compositions can be formulated for delivery to the eye, e.g., in the form of a pharmaceutically acceptable solution, suspension or ointment. A preparation for use in treating an eye can be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. A preparation as described herein can be administered topically to the eye of the subject in need of treatment (e.g., a subject afflicted with AMD) by conventional methods, e.g., in the form of drops, or by bathing the eye in a therapeutic solution, containing one or more compositions.

A variety of devices for introducing drugs into the vitreal cavity of the eye may be appropriate, in certain embodiments, for administration of a composition as described herein. For example, U.S. Publication No. 2002/0026176 describes a pharmaceutical-containing plug that can be inserted through the sclera such that it projects into the vitreous cavity to deliver the pharmaceutical agent into the vitreous cavity. In another example, U.S. Pat. No. 5,443,505 describes an implantable device for introduction into a suprachoroidal space or an avascular region for sustained release of drug into the interior of the eye. U.S. Pat. Nos. 5,773,019 and 6,001,386 each disclose an implantable drug delivery device attachable to the scleral surface of an eye. Additional methods and devices (e.g., a transscleral patch and delivery via contact lenses) for delivery of a therapeutic agent to the eye are described in, e.g., Ambati and Adamis (2002) Prog Retin Eye Res 21(2):145-151; Ranta and Urtti (2006) Adv Drug Delivery Rev 58(11):1164-1181; Barocas and Balachandran (2008) Expert Opin Drug Delivery 5(1):1-10(10); Gulsen and Chauhan (2004) Invest Opthalmol Vis Sci 45:2342-2347; Kim et al. (2007) Ophthalmic Res 39:244-254; and PCT publication no. WO 04/073551, the disclosures of which are incorporated herein by reference in their entirety.

In certain embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407). Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc Natl Acad Sci USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically pre-filled with one or more therapeutic unit doses of a therapeutic solution, and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition described herein can be locally administered to a joint (e.g., an articulated joint). For example, in embodiments where the disorder is arthritis, a therapeutically appropriate composition can be administered directly to a joint (e.g., into a joint space) or in the vicinity of a joint. Examples of intraarticular joints to which a composition described herein can be locally administered include, e.g., the hip, knee, elbow, wrist, sternoclavicular, temperomandibular, carpal, tarsal, ankle, and any other joint subject to arthritic conditions. A composition described herein can also be administered to bursa such as, e.g., acromial, bicipitoradial, cubitoradial, deltoid, infrapatellar, ischial, and any other bursa known in the art of medicine.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody as described herein may be required to treat a subject with RA as compared to the dose of a different formulation of that antibody. Other factors affecting the dose administered to the subject include, e.g., the type or severity of the disorder. For example, a subject having RA may require administration of a different dosage than a subject with PNH. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A composition described herein can be administered as a fixed dose, or in a milligram per kilogram (mg/kg) dose. In some embodiments, the dose can also be chosen to reduce or avoid production of antibodies or other host immune responses against one or more of the antigen-binding molecules in the composition. While in no way intended to be limiting, exemplary dosages of an antibody, such as a composition described herein include, e.g., 1-1000 mg/kg, 1-100 mg/kg, 0.5-50 mg/kg, 0.1-100 mg/kg, 0.5-25 mg/kg, 1-20 mg/kg, and 1-10 mg/kg. Exemplary dosages of a composition described herein include, without limitation, 0.1 mg/kg, 0.5 mg/kg, 1.0 mg/kg, 2.0 mg/kg, 4 mg/kg, 8 mg/kg, or 20 mg/kg.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of the complement-mediated disorder. For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of compositions can be determined by known pharmaceutical procedures in cell cultures or experimental animals (e.g., animal models of any of the complement-mediated disorders described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

### Combination Therapies

In various embodiments, an engineered antibody as described herein may be included in a course of treatment that further includes administration of at least one additional agent to a subject. In various instances, an additional agent administered in combination with an engineered antibody as described herein may be an agent that inhibits complement, e.g., an agent that inhibits terminal compliment. In various instances, an additional agent administered in combination with an antibody as described herein may be an agent that inhibits inflammation. In various instances, an additional agent administered in combination with an antibody as described herein may be an agent that treats a symptom of PNH. In various instances, an additional agent administered in combination with an antibody as described herein may be an agent that treats a symptom of aHUS.

In some embodiments, the methods can be performed in conjunction with other therapies for complement-associated disorders. For example, the composition can be administered to a subject at the same time, prior to, or after, plasmapheresis, IVIG therapy, or plasma exchange. See, e.g., Appel et al. (2005) J Am Soc Nephrol 16:1392-1404. In some embodiments, the composition can be administered to a subject at the same time, prior to, or after, a kidney transplant.

In various instances, an additional agent administered in combination with an engineered antibody as described herein may be administered at the same time as an engineered antibody, on the same day as an engineered antibody, or in the same week as an engineered antibody. In various instances, an additional agent administered in combination with an engineered antibody as described herein may be administered in a single formulation with an engineered antibody. In certain embodiments, an additional agent administered in a manner temporally separated from administration of an engineered antibody as described herein, e.g., one or more hours before or after, one or more days before or after, one or more weeks before or after, or one or more months before or after administration of an engineered antibody. In various embodiments, the administration frequency of one or more additional agents may be the same as, similar to, or different from the administration frequency of an engineered antibody as described herein.

Encompassed within combination therapy is the a treatment regimen that includes administration of two distinct antibodies as described herein and/or a treatment regimen that includes administration of an antibody as described herein by a plurality of formulations and/or routes of administration.

In some embodiments, compositions can be formulated with one or more additional therapeutic agents, e.g., additional therapies for treating or preventing a complement-associated disorder (e.g., an AP-associated disorder or a CP-associated disorder) in a subject. Additional agents for treating a complement-associated disorder in a subject will vary depending on the particular disorder being treated, but can include, without limitation, an antihypertensive (e.g., an angiotensin-converting enzyme inhibitor) [for use in treating, e.g., HELLP syndrome], an anticoagulant, a corticosteroid (e.g., prednisone), or an immunosuppressive agent (e.g., vincristine or cyclosporine A). Examples of anticoagulants include, e.g., warfarin (Coumadin), aspirin, heparin, phenindione, fondaparinux, idraparinux, and thrombin inhibitors (e.g., argatroban, lepirudin, bivalirudin, or dabigatran). A composition described herein can also be formulated with a fibrinolytic agent (e.g., ancrod, E-aminocaproic acid, antiplasmin-aᵢ prostacyclin, and defibrotide) for the treatment of a complement-associated disorder. In some embodiments, a composition can be formulated with a lipid-lowering agent such as an inhibitor of hydroxymethylglutaryl CoA reductase. In some embodiments, a composition can be formulated with, or for use with, an anti-CD20 agent such as rituximab (RITUXAN™; Biogen Idec, Cambridge, Mass.). In some embodiments, e.g., for the treatment of RA, the composition can be formulated with one or both of infliximab (REMICADE®; Centocor, Inc.) and methotrexate (RHEUMATREX®, TREXALL®). In some embodiments, a composition described herein can be formulated with a non-steroidal anti-inflammatory drug (NSAID). Many different NSAIDS are available, some over the counter including ibuprofen (ADVIL®, MOTRIN®, NUPRIN®) and naproxen (ALLEVE®) and many others are available by prescription including meloxicam (MOBIC®), etodolac (LODINE®), nabumetone (RELAFEN®), sulindac (CLINORIL®), tolementin (TOLECTIN®), choline magnesium salicylate (TRILASATE®), diclofenac (CATAFLAM®, VOLTAREN®, ARTHROTEC®), Diflusinal (DOLOBID®), indomethicin (INDOCIN®Ketoprofen (ORUDIS®, ORUVAIL®), Oxaprozin (DAYPRO®), and piroxicam (FELDENE®). In some embodiments a composition can be formulated for use with an anti-hypertensive, an anti-seizure agent (e.g., magnesium sulfate), or an anti-thrombotic agent. Anti-hypertensives include, e.g., labetalol, hydralazine, nifedipine, calcium channel antagonists, nitroglycerin, or sodium nitroprussiate. (See, e.g., Mihu et al. (2007) J Gastrointestin Liver Dis 16(4):419-424.) Anti-thrombotic agents include, e.g., heparin, antithrombin, prostacyclin, or low dose aspirin.

In some embodiments, compositions including an engineered antibody as described herein can be formulated for administration with one or more additional therapeutic agents for use in treating a complement-associated disorder of the eye. Such additional therapeutic agents can be, e.g., bevacizumab or the Fab fragment of bevacizumab or ranibizumab, both sold by Roche Pharmaceuticals, Inc., and pegaptanib sodium (MUCOGEN®; Pfizer, Inc.). Such a kit can also, optionally, include instructions for administering the composition to a subject.

In some examples, the combination therapy can include administering to the subject one or more additional agents (e.g., an anti-IgE antibody, an anti-IL-4 antibody, an anti-IL-5 antibody, or an anti-histamine) that provide therapeutic benefit to a subject who has, is at risk of developing, or is suspected of having a complement-associated pulmonary disorder such as COPD or asthma.

In some embodiments, compositions formulated for intrapulmonary administration can include at least one additional active agent for treating a pulmonary disorder. The at least one active agent can be, e.g., an anti-IgE antibody (e.g., omalizumab), an anti-IL-4 antibody or an anti-IL-5 antibody, an anti-IgE inhibitor (e.g., montelukast sodium), a sympathomimetic (e.g., albuterol), an antibiotic (e.g., tobramycin), a deoxyribonuclease (e.g., PULMOZYME®), an anticholinergic drug (e.g., ipratropium bromide), a corticosteroid (e.g., dexamethasone), a β-adrenoreceptor agonist, a leukotriene inhibitor (e.g., zileuton), a 5-lipoxygenase inhibitor, a PDE inhibitor, a CD23 antagonist, an IL-13 antagonist, a cytokine release inhibitor, a histamine H1 receptor antagonist, an anti-histamine, an anti-inflammatory agent (e.g., cromolyn sodium), or a histamine release inhibitor.

In some embodiments, compositions can be formulated for administration to a subject along with intravenous gamma globulin therapy (IVIG), plasmapheresis, plasma replacement, or plasma exchange. In some embodiments, compositions can be formulated for use before, during, or after, a kidney transplant.

When compositions are to be used in combination with a second active agent, the compositions can be co-formulated with the second agent or the compositions can be formulated separately from the second agent formulation. For example, the respective pharmaceutical compositions can be mixed, e.g., just prior to administration, and administered together or can be administered separately, e.g., at the same or different times.

A composition described herein can replace or augment a previously or currently administered therapy. For example, upon treating with a composition described herein, administration of the one or more additional active agents can cease or diminish, e.g., be administered at lower levels, e.g., lower levels of a reference antibody comprising a heavy chain having the amino acid sequence of SEQ ID NO: 1 and a light chain having the amino acid sequence of SEQ ID NO: 2 following administration of an engineered antibody described herein. In some embodiments, administration of the previous therapy can be maintained. In some embodiments, a previous therapy will be maintained until the level of the composition reaches a level sufficient to provide a therapeutic effect. The two therapies can be administered in combination.

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered antibody described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Engineering Anti-C5 Antibodies with Improved pH Switch

The present Example demonstrates selection and engineering of therapeutic antibodies against C5 with improved pharmacologic and pharmacodynamics properties.

In the present Example, improved anti-C5 antibodies based on a reference antibody comprising a heavy chain having the amino acid sequence of SEQ ID NO: 1 and a light chain having the amino acid sequence of SEQ ID NO: 2 were engineered to include mutations that enhance pH-dependent binding affinity to C5.

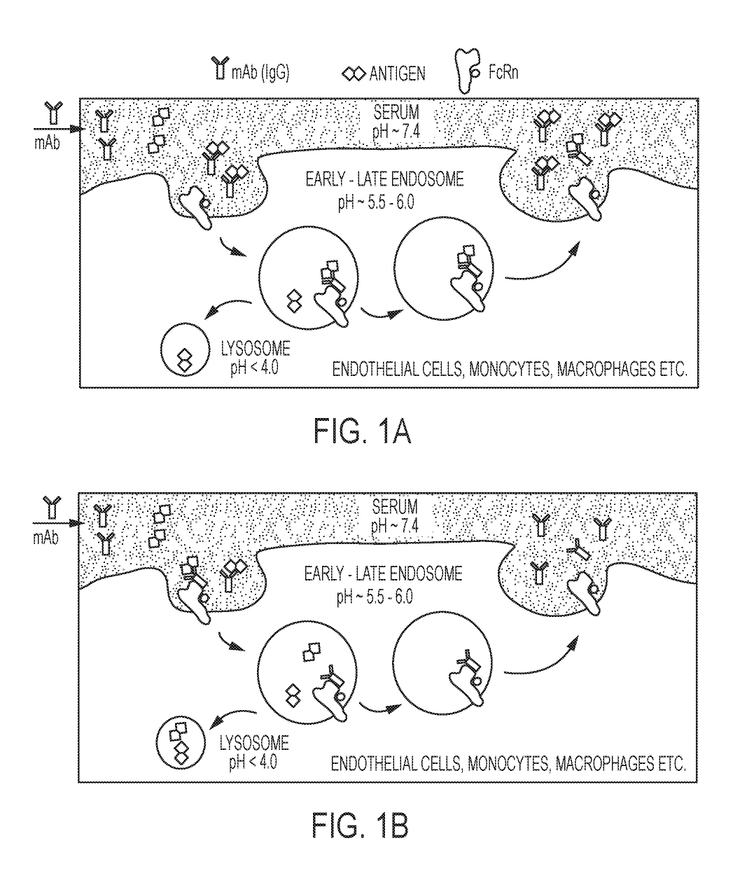

FIGS. 1A and 1B illustrate an exemplary scheme to build antibodies with improved pharmacologic and pharmacodynamics properties. A non-engineered anti-C5 antibody, such as an antibody comprising a heavy chain having the amino acid sequence of SEQ ID NO: 1 and a light chain having the amino acid sequence of SEQ ID NO: 2, binds serum complement C5 protein and inactivates it. However, serum C5 is bound up in a C5-antibody complex. Non-engineered anti-C5 antibodies have a typical antibody half-life because they are recycled by FcRn.

As shown in FIG. 1B, the engineered antibodies bind to C5 in serum typically at a neutral pH (e.g., above pH 7.4). Once endocytosed, the pH starts to decrease. In the early endosome, when the pH drops to about 6.0 or below, the antibodies bind FcRn with high affinity via the Fc domain and at the same time, the antibody dissociates from antigen (C5). As a result, the antibodies are recycled out by FcRn and the antigen C5 is degraded in lysosomes. This process improves the “processivity” of the antibodies, as described herein, thereby allowing these antibodies to have better pharmacokinetic and pharmacodynamic profiles as compared to the reference antibody.

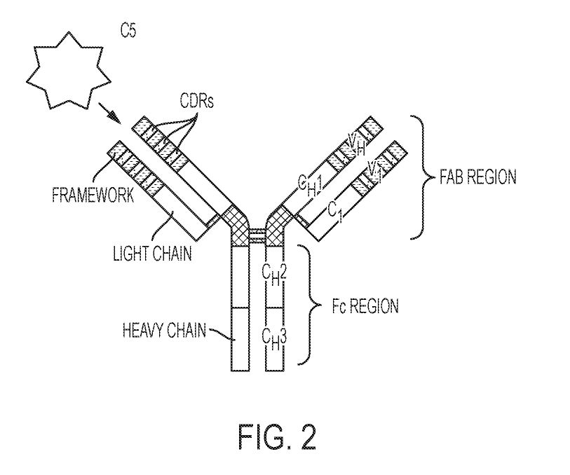

FIG. 2 shows a schematic illustration of the basic structural components of an anti-C5 antibody.

Specifically, histidine mutations were systemically introduced in the heavy chain variable region (amino acid residues 1-122 of SEQ ID NO:1) and in the light chain variable region (amino acid residues 1-108 of SEQ ID NO: 2) to engineer improved antibodies that bind C5 with higher affinity in serum (at a neutral pH, e.g., at or above pH 7.4) relative to that in endosomes (at an acidic pH, e.g., at or below pH 6.0 such as pH 5.5). In other words, the antigen binding region was engineered to unbind C5 in endosomes, which is also referred to as pH-labile antigen binding, pH-dependent switch, or pH-switch. Selected histidine mutations were further validated in an IgG1 antibody backbone (i.e., an anti-C5 antibody with an IgG1 Fc constant region).

As shown in Table 3 below, histidine substitution of phenylalanine at position 100 of the heavy chain variable region (amino acid residues 1-122 of SEQ ID NO:1) and histidine substitution of serine at position 26 of the light chain variable region (amino acid residues 1-108 of SEQ ID NO:2) renders unexpectedly superior pH-dependent switch of the engineered anti-C5 antibodies (˜14-fold) as compared to the reference (Ref.) antibody (˜2-fold). The binding kinetics between anti-C5 antibodies and human C5 (hC5) were measured by Biacore® assays at 7.4 and pH 5.5 using immobilized mAbs (via capture with anti-Fc antibodies).

As used herein, an antibody variable region with F100H substitution in the heavy chain (corresponding to amino acid residues 1-122 of SEQ ID NO:1) and S26H substitution in the light chain variable region (amino acid residues 1-108 of SEQ ID NO:2) is also referred to as the F100-S26 backbone. As shown in Table 3, one or more additional mutations (e.g., histidine substitutions) on the F100-S26 backbone further enhance pH-switch. Furthermore, the same mutation (e.g., histidine substitution) on the F100-S26 backbone results in greater pH-switch as compared to that on the reference backbone. In other words, exemplary results shown in Table 3 demonstrate that the F100-S26 back bone in combination with one or more additional mutations (e.g., one or more histidine substitutions) has synergistic effects on pH-switch. Additional mutants generated include histidine substitutions in the heavy chain at positions 33, 35, 50, 52, 53, 99, 101, 105, 107, 108, 109, 110, and in the light chain at positions 32, 33, 34, 50, and 96 (data not shown).

TABLE 3
F100H and S26H combination facilitates pH-Switch

| Kd pH 7.4 (M) | | ΔKd Ref./Mt. | | Kd pH 5.5/7.4 | |
|---|---|---|---|---|---|
| Ref. | F100-S26 | Ref. | F100-S26 | Ref. | F100-S26 |

Heavy Chain

| | | | | | | |
|---|---|---|---|---|---|---|
| Ref. | 9.91E−10 | 1.07E−09 | 100% | 100% | 2.6 | 14.69 |
| G26H | 1.05E−09 | 1.51E−09 | 94% | 71% | 4.91 | 27.82 |
| Y27H | 3.91E−09 | 2.84E−09 | 25% | 38% | 10.99 | 41.23 |
| I28H | 1.56E−09 | 9.31E−10 | 64% | 115% | 3.42 | 23.38 |
| F29H | 7.42E−10 | 1.59E−09 | 134% | 67% | 8.90 | 23.17 |
| S30H | 1.30E−09 | 9.11E−10 | 76% | 117% | 3.3 | 23.08 |
| N31H | 1.39E−09 | 6.76E−10 | 71% | 158% | 3.15 | 17.85 |
| Y32H | 1.20E−09 | 1.34E−09 | 83% | 80% | 7.84 | 21.38 |
| I34H | 2.89E−09 | 2.98E−09 | 34% | 36% | 8.51 | 37.57 |
| I51H | 1.63E−09 | 9.24E−10 | 61% | 116% | 4.2 | 767.33 |
| G54H | 1.46E−09 | 1.05E−09 | 68% | 102% | 3.27 | 21.60 |
| S55H | 1.04E−09 | 1.55E−09 | 95% | 69% | 6.67 | 24.2 |
| G56H | 1.33E−09 | 1.50E−09 | 75% | 71% | 2.83 | 14.3 |
| S57H | 1.67E−09 | 1.70E−09 | 59% | 63% | 7.54 | 38.66 |
| T58H | 8.76E−10 | 1.05E−09 | 113% | 102% | 3.66 | 11.7 |
| E59H | 4.13E−08 | 1.41E−08 | 2% | 8% | 16.08 | 2.13 |
| Y60H | 1.83E−09 | 2.19E−09 | 54% | 49% | 4.97 | 26.09 |
| T61H | 1.51E−09 | 1.74E−09 | 66% | 61% | 8.35 | 26.4 |
| E62H | 1.14E−09 | 8.34E−10 | 87% | 128% | 2.36 | 9.67 |
| N63H | 1.47E−09 | 9.73E−10 | 67% | 110% | 1.83 | 10.03 |
| F64H | 1.23E−09 | 1.96E−09 | 81% | 55% | 5.47 | 20.78 |
| K65H | 1.03E−09 | 7.94E−10 | 96% | 135% | 2.85 | 13.99 |
| D66H | 1.26E−09 | 1.01E−09 | 79% | 106% | 2.57 | 11.66 |
| F100H | 1.34E−09 | NA | 74% | F100H | 6.8 | NA |
| G102H | 1.84E−09 | 2.54E−09 | 54% | 42% | 14.60 | 37.06 |
| S103H | 1.88E−09 | 2.36E−09 | 53% | 45% | 5.87 | 23.77 |
| S104H | 3.78E−09 | 3.44E−09 | 26% | 31% | 2.53 | 6.73 |
| N106H | 1.22E−09 | 9.76E−10 | 81% | 110% | 3.06 | 7.71 |
| V111H | 1.06E−09 | 9.92E−10 | 93% | 108% | 5.07 | 19.62 |

Light Chain

| | | | | | | |
|---|---|---|---|---|---|---|
| G24H | 1.20E−09 | 9.92E−10 | 83% | 108% | 3.09 | 19.40 |
| A25H | 1.54E−09 | 1.07E−09 | 64% | 100% | 3.11 | 15.89 |
| S26H | 9.41E−10 | 1.39E−09 | 105% | S26H | 4.01 | NA |
| E27H | 1.69E−09 | 1.41E−09 | 59% | 76% | 4.09 | 13.16 |
| N28H | 1.39E−09 | 1.18E−09 | 71% | 91% | 2.49 | 14.79 |
| I29H | 1.17E−09 | 3.21E−09 | 85% | 33% | 2.52 | 19.4 |
| Y30H | 1.48E−09 | 1.43E−09 | 68% | 75% | 4.41 | 19.12 |
| G31H | 3.21E−09 | 4.08E−09 | 31% | 26% | 11.52 | 50.76 |
| A51H | 9.83E−10 | 1.09E−09 | 101% | 98% | 1.8 | 4.02 |
| T52H | 1.44E−09 | 1.45E−09 | 69% | 74% | 2.73 | 12.09 |
| N53H | 9.13E−10 | 7.95E−10 | 109% | 135% | 3.43 | 16.36 |
| L54H | 1.07E−09 | 1.15E−09 | 93% | 93% | 3.06 | 13.47 |
| A55H | 1.25E−09 | 1.01E−09 | 79% | 106% | 2.57 | 9.16 |
| D56H | 1.22E−09 | 1.17E−09 | 81% | 91% | 3.74 | 21.26 |
| Q89H | 1.28E−09 | 2.48E−09 | 77% | 43% | 3.43 | 9.61 |
| N90H | 1.48E−09 | 1.23E−09 | 67% | 87% | 2.82 | 12.29 |
| V91H | 9.97E−09 | 5.43E−09 | 10% | 20% | 12.33 | 16.46 |
| L92H | 1.18E−08 | 1.77E−08 | 8% | 6% | 20.27 | 58.76 |
| N93H | 1.58E−09 | 1.52E−09 | 62% | 70% | 5.48 | 17.83 |
| T94H | 9.18E−09 | 7.73E−09 | 11% | 14% | 17.49 | 8.86 |
| P95H | 2.76E−09 | 3.12E−09 | 36% | 34% | 6.04 | 11.06 |
| T97H | 9.80E−10 | 8.10E−10 | 101% | 132% | 2.97 | 8.77 |

Exemplary histidine substitutions as shown in Table 4 were selected for further analysis.

TABLE 4
Exemplary histidine substitutions selected for further analysis

| Kd pH 7.4 (M) | | ΔKd Ref./Mt. | | Kd pH 5.5/7.4 | |
|---|---|---|---|---|---|
| Ref. | F100-S26 | Ref. | F100-S26 | Ref. | F100-S26 |

Heavy Chain

| | | | | | | |
|---|---|---|---|---|---|---|
| Ref. | 9.91E−10 | 1.07E−09 | 100% | 100% | 2.6 | 14.69 |
| Y27H | 3.91E−09 | 2.84E−09 | 25% | 38% | 10.99 | 41.23 |
| F29H | 7.42E−10 | 1.59E−09 | 134% | 67% | 8.90 | 23.17 |
| Y32H | 1.20E−09 | 1.34E−09 | 83% | 80% | 7.84 | 21.38 |
| I34H | 2.89E−09 | 2.98E−09 | 34% | 36% | 8.51 | 37.57 |
| I51H | 1.63E−09 | 9.24E−10 | 61% | 116% | 4.2 | 767.33 |
| S55H | 1.04E−09 | 1.55E−09 | 95% | 69% | 6.67 | 24.2 |
| S57H | 1.67E−09 | 1.70E−09 | 59% | 63% | 7.54 | 38.66 |
| T61H | 1.51E−09 | 1.74E−09 | 66% | 61% | 8.35 | 26.4 |
| F64H | 1.23E−09 | 1.96E−09 | 81% | 55% | 5.47 | 20.78 |
| F100H | 1.34E−09 | NA | 74% | F100H | 6.8 | NA |
| G102H | 1.84E−09 | 2.54E−09 | 54% | 42% | 14.60 | 37.06 |
| S103H | 1.88E−09 | 2.36E−09 | 53% | 45% | 5.87 | 23.77 |

Light Chain

| | | | | | | |
|---|---|---|---|---|---|---|
| S26H | 9.41E−10 | 1.39E−09 | 105% | S26H | 4.01 | NA |
| G31H | 3.21E−09 | 4.08E−09 | 31% | 26% | 11.52 | 50.76 |
| D56H | 1.22E−09 | 1.17E−09 | 81% | 91% | 3.74 | 21.26 |
| V91H | 9.97E−09 | 5.43E−09 | 10% | 20% | 12.33 | 16.46 |
| L92H | 1.18E−08 | 1.77E−08 | 8% | 6% | 20.27 | 58.76 |
| N93H | 1.58E−09 | 1.52E−09 | 62% | 70% | 5.48 | 17.83 |
| P95H | 2.76E−09 | 3.12E−09 | 36% | 34% | 6.04 | 11.06 |

### Example 2

### IgG1 Backbone Enhances pH-Switch

This example illustrates that the IgG1 backbone unexpectedly enhances pH-switch, as compared to the IgG2 backbone commonly used in the prior art.

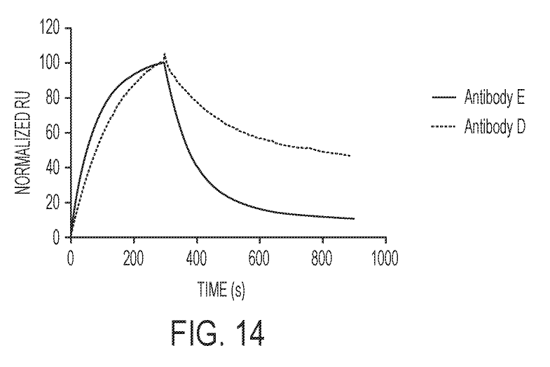

Specifically, histidine substitution of phenylalanine at position 100 of the heavy chain variable region (amino acid residues 1-122 of SEQ ID NO:1) and histidine substitution of asparagine at position 93 of the light chain variable region (amino acid residues 1-108 of SEQ ID NO:2) were introduced onto either an IgG1 (Antibody E comprising SEQ ID NOs:435 and 463) or IgG2 (Antibody D comprising SEQ ID NOs: 462 and 463) backbone (i.e., same HC and LC variable regions with an IgG1 Fc constant region or IgG2 Fc constant region, respectively). Unexpectedly, the antibody with the IgG1 Fc constant region (Antibody E) demonstrated significantly higher fold change in C5 binding affinity at pH5.5 vs. pH7.4 as compared to the antibody with the IgG2 Fc constant region (48.47 fold vs. 13.4 fold) (see Table 5). Similarly, Antibody E (with the IgG1 Fc constant region) has weaker binding and faster dissociation at pH 5.5 as compared to Antibody D (with the IgG2 Fc constant region), as shown in FIG. 14.

TABLE 5
IgG1 backbone enhances pH-switch

| | | Kd at pH 7.4 | Kd at pH 5.5 | Fold Change in Kd |
|---|---|---|---|---|
| | Fc | (nM) | (nM) | (pH 7.4 vs. pH 5.5) |
| Antibody D | IgG2 | 1.04 | 13.9 | 13.37 |
| Antibody E | IgG1 | 0.72 | 34.9 | 48.47 |

These results indicate that an IgG1 Fc constant region (also referred to as an IgG1 backbone) significantly enhances pH-switch, as compared to an IgG2 Fc constant region (also referred to as an IgG2 backbone). Thus, without wishing to be bound by a particular theory, the use of an IgG1 backbone in validating anti-C5 histidine mutants as described in Example 1 above may have unexpectedly enhanced the pH-switch effect, resulting in identification of the novel mutations described herein that otherwise could have been missed if a different IgG subtype (e.g., IgG2 or IgG4) backbone was used.

### Example 3

### Engineered Anti-C5 Antibodies with Improved Pharmacology and Pharmacokinetics and Reduced Effector Functions

This example illustrates exemplary engineered anti-C5 antibodies according to the present invention with improved pharmacologic and pharmacodynamics properties, as well as reduced effector functions.

Specifically, Antibody A, Antibody B, and Antibody C were engineered tothe present invention and as detailed in Table 6 below. In Table 6, an “X” in a box indicates that for a given engineered antibody, one or more residues in the indicated CDRs were mutated to histidine. Antibody A has a light chain comprising SEQ ID NO: 453 (including the variable region corresponding to SEQ ID NO:311) and a heavy chain comprising SEQ ID NO: 460 (including the variable region corresponding to SEQ ID NO:222 and the Fc constant region corresponding to SEQ ID NO:429). Antibody B has a light chain comprising SEQ ID NO: 453 (including the variable region corresponding to SEQ ID NO:311) and a heavy chain comprising SEQ ID NO: 452 (including the variable region corresponding to SEQ ID NO:222 and the Fc constant region corresponding to SEQ ID NO:431). Antibody C has a light chain comprising SEQ ID NO:457 (including the variable region corresponding to SEQ ID NO:308) and a heavy chain comprising SEQ ID NO:452 (including the variable region corresponding to SEQ ID NO:222 and the Fc constant region corresponding to SEQ ID NO:431).

Table 6 also lists antibodies “Control 1” and “Control 2” used as controls in experiments. Control 1 is an antibody whose heavy chain has the amino acid sequence of SEQ ID NO: 1 and whose light chain has the amino acid sequence of SEQ ID NO: 2. Control 2 is an antibody whose light chain has the amino acid sequence of SEQ ID NO: 2 and whose heavy chain variable region has the amino acid sequence of amino acids 1-122 of SEQ ID NO: 1 . Control 2 has an IgG1 heavy chain constant region with mutations at L234 and L235 (SEQ ID NO: 429).

In addition, Antibody A, Antibody B and Antibody C were engineered to include the indicated mutations in the Fc region. Without wishing to be bound by any theory, it is believed that such mutations reduce or remove effector functions.

Antibody B was further engineered to include mutations at residues H433 and N434 of the Fc domain, as show in Table 6. Without wishing to be bound by any theory, it is believed that such engineering of the Fc domain allows binding to FcRn with higher affinity in endosomes (at an acidic pH, e.g., at or below pH 6.0) relative to that in serum (at a neutral pH, e.g., at or above pH 7.4) such that the antibodies can be salvaged by FcRn in the endosomes and recycled back to the serum, as shown in FIG. 1. Such enhanced FcRn recycling may result in and/or contribute to increased antibody half-life.

TABLE 6
Mutations in engineered antibodies
(relative to a reference antibody comprising a heavy chain having the amino acid sequence of SEQ
ID NO: 1 and a light chain having the amino acid sequence of SEQ ID NO: 2)

| | Regions | | | | | | | | |
|---|---|---|---|---|---|---|---|---|---|
| Antibody | Heavy chain | | | Light chain | | | | | IgG |
| name | CDR1 | CDR2 | CDR3 | CDR1 | CDR2 | CDR3 | FW | Fc | subclass |
| Control 1 | | | | | | | | | IgG2 |
| | | | | | | | | | and |
| | | | | | | | | | IgG4 |
| Control 2 | | | | | | | | L234A | IgG1 |
| | | | | | | | | L235A | |
| Antibody A | X | | X | X | X | | I28T | L234A | IgG1 |
| | | | | | | | T76I | L235A | |
| | | | | | | | V79A | | |
| Antibody B | X | | X | X | X | | I28T | L234A | IgG1 |
| | | | | | | | T76I | L235A | |
| | | | | | | | V79A | H433K | |
| | | | | | | | | N434F | |
| Antibody C | X | | X | X | X | | I28T | L234A | IgG1 |
| | | | | | | | T76I | L235A | |
| | | | | | | | V79A | H433K | |
| | | | | | | | | N434F | |

X's indicate mutations of one or more residues to histidine.
EU numbering is used for the positions of residues.

Antibody A, Antibody B and Antibody C were produced in human embryonic kidney (HEK 293) cells or Chinese Hamster Ovary (CHO) cells by transient expression of a construct for the full-length antibody. Control 1 was produced in CHO cells for use as a control in experiments. Control 2 was produced in HEK 293 cells for use as a control in experiments.

### Example 4

### pH-Differential Binding of Engineered Anti-C5 Antibodies to C5

It was believed that depletion of C5 from serum could be enhanced by dissociation of anti-C5 antibody/C5 complex in the acidic environment of the endosome (pH 5.5 to 6.5). The present Example illustrates that engineered antibodies of the present invention have enhanced dissociation of anti-C5 antibody/C5 complex in the acidic environment of the endosome (pH 5.5 to 6.5).

The kinetics of binding of purified human C5 (hC5) or hC5 in human serum to immobilized mAbs (via capture with anti-Fc antibodies) were measured by Biacore® assays over a pH range from 7.4 to 5.5. Binding was tested at pH 7.4, 7.0, 6.5, 6.0, and 5.5. Changes in affinity with changes in pH were gradual, with significant differences noted at pH 6.5.

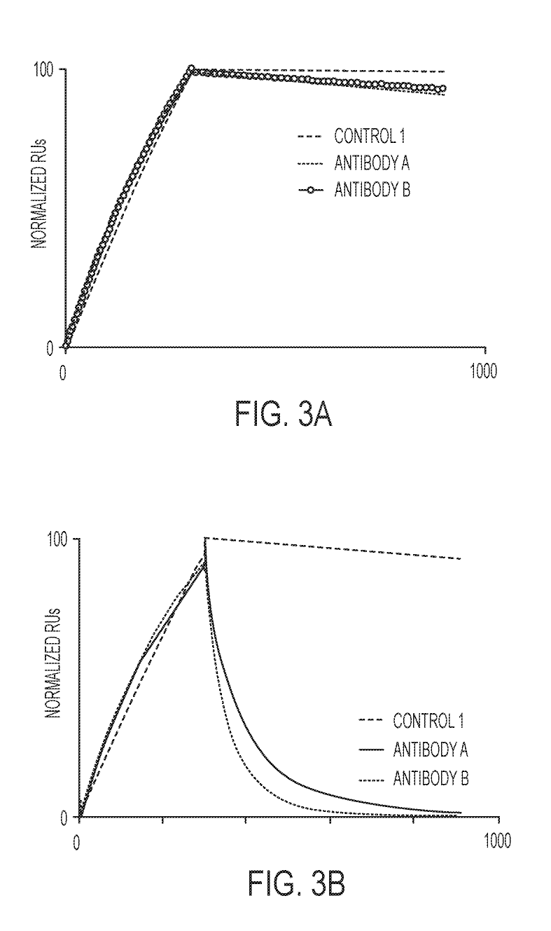

FIGS. 3A and 3B show representative binding curves over time, with the association phase at pH 7.4 and the dissociation phase at pH 7.4 (FIG. 3A) or at pH 5.5 (FIG. 3B) for Antibody A, Antibody B, and Control 1 to purified hC5. Table 7 below summarizes the measured K_{D} values at pH 7.4 and pH 5.5 and fold-changes in K_{D} measured at pH 7.4 versus at pH 5.5 for Antibody A, Antibody B, and Antibody C.

TABLE 7
Dissociation constants (K_{D}) and fold-changes
for engineered anti-C5 antibodies

| | | |
|---|---|---|
| IgG | K_{D} (nM) | K_{D} |

| | subclass | pH 7.4 | pH 5.5 | fold-change |
|---|---|---|---|---|
| Control 1 | 2-4 | 0.12 | 1.46 | 12.17 |
| Antibody A | 1 | 0.34 | 113 | 332 |
| Antibody B | 1 | 0.35 | 145 | 414 |
| Antibody C | 1 | 0.26 | 37 | 142 |

As shown in Table 7, Antibody A exhibited an approximately 332-fold change. Antibody B exhibited an approximately 414-fold change in the K_{D} value at pH 5.5 versus at pH 7.4. Antibody C exhibited an approximately 142-fold change in the K_{D} value at pH 5.5 versus at pH 7.4. This greatly enhanced pH-differential binding has not been previously observed with other anti-C5 antibodies known in the art. Significantly, these engineered anti-C5 antibodies also retained their binding affinity to C5 at pH 7.4 which is clinically important. The enhanced pH-differential binding resulted in less than 3-fold reduction of the C5 binding affinity at pH 7.4 as compared to Control 1.

### Example 5

### pH-Differential Binding of Engineered Anti-C5 Antibodies to FcRn

The present Example demonstrates that an engineered anti-C5 antibody of the present invention exhibited increased binding to FcRn at acidic pH.

Kinetics of binding to immobilized recombinant FcRn was determined for mAbs at pH 7.4 and pH 5.5 in Biacore® assays. Both Antibody A and Control 1 showed no detectable binding to human FcRn (hFcRn) at pH 7.4, while Antibody B showed an increased binding affinity for hFcRn in a pH-dependent manner

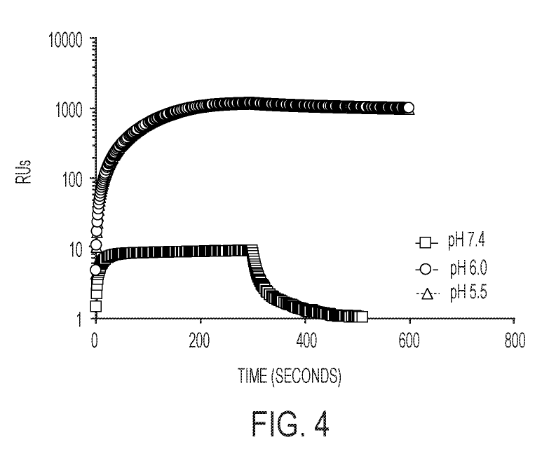

FIG. 4 shows representative binding curves over time at pH 7.4, pH 6.0, and pH 5.5 for Antibody B plotted on a semi-log scale. Binding to hFcRn was increased over many fold, with a change in pH from 7.4 to 6.0. Further acidification to pH 5.5 did not measurably increase affinity. Table 8 summarizes data regarding affinity to FcRn.

TABLE 8
Affinity to purified hFcRn at different pH levels

| | K_{D} (nM) | | |
|---|---|---|---|
| Antibody | pH 7.4 | pH 6.0 | pH 5.5 |
| Antibody A | No binding in | 11.3 | 5.9 |
| | range tested | | |
| Antibody B | 108 (50-0.39 nM) | 0.24 | 0.13 |
| | 191 (2000-15.6 nM) | | |
| Control 1 | No binding in | 125 | 125 |
| | range tested | | |

### Example 6

### Modulation of the Complement System using Engineered Anti-C5 Antibodies

The present Example demonstrates that Antibody A and Antibody B effectively inhibited complement system activation, as assessed by two assays.

The first assay was based on the fact that classical complement pathway activation results in lysis of red blood cells (RBCs); therefore, the extent of inhibition of RBCs is a functional indicator of inhibition of complement activity. The second assay examines the formation of the terminal complement complex (TCC, also known as the membrane attack complex (MAC) and as C5b-9), which occurs downstream of C5 in the complement activation pathway. Formation of the MAC is an indicator of the availability of free C5 and is correlated with efficacy outcomes in diseases such as PNH.

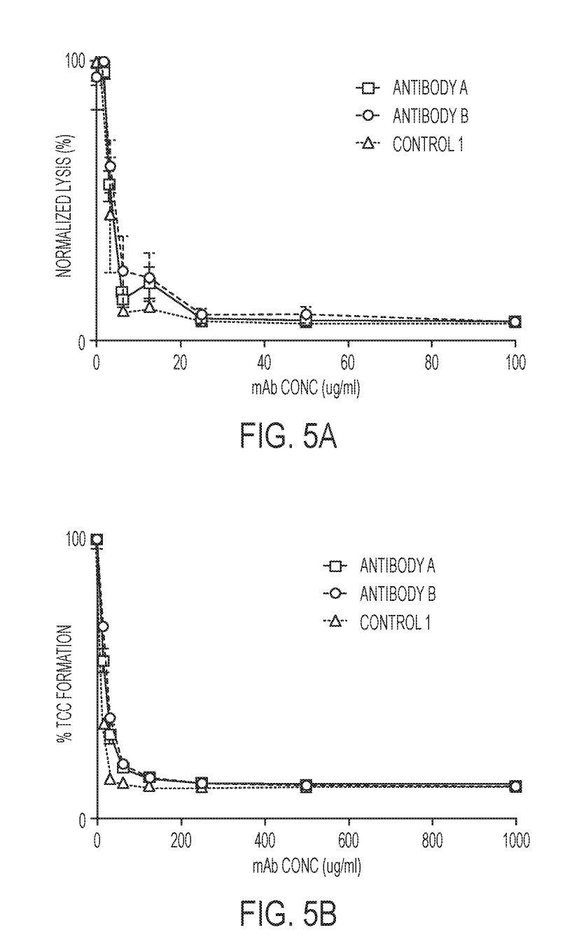

RBC assays were performed using chicken RBCs incubated with human serum. FIG. 5A shows the results of RBC lysis inhibition assays for Antibody A and Antibody B, with Control 1 as the control antibody. Both Antibody A and Antibody B inhibited RBC lysis in a concentration-dependent manner.

The IC₅₀ values for inhibition of antibody-sensitized lysis of chicken RBC by human serum were comparable for all antibodies tested; it was 3 μg/mL for Antibody A, Antibody B, and Control 1. No lysis was detected when C5-depleted serum was used (data not shown).

Formation of the TCC was studied with samples incubated with engineered anti-C5 antibodies (Antibody A and Antibody B) or with Control 1 as a control. As shown in FIG. 5B, comparable inhibition of TCC formation was observed for Antibody A (24 μg/mL), Antibody B (20 μg/mL), and Control 1 (24 μg/mL). These results indicate that engineered anti-C5 antibodies of the present invention maintain the ability to potently inhibit complement system activation downstream of C5.

### Example 7

### Reduced Effector Function in Engineered Anti-C5 Antibodies

Binding and functional assays were performed to verify that the L234A and L235A mutations introduced in the IgG1 Fc region can reduce effector function. As shown in Table 9, binding to Fcγreceptors was reduced for both Antibody A and Antibody B.

TABLE 9
Binding affinities to Fcγ receptors

| | FcγRI | FcγRIIA | FcγRIIIA | FcγRIIIB |
|---|---|---|---|---|
| Antibody | (nM) | (nM) | (nM) | (nM) |
| (Ranges | (62.5-0.48 | (1000-31.25 | (1000-31.25 | (1000-31.25 |
| tested) | nM) | nM) | nM) | nM) |
| Antibody A | 82/54 | No binding | 7420 | No binding |
| | | in range | | in range |
| | | tested | | tested |
| Antibody B | 94/75/62 | No binding | 9620 | No binding |
| | | in range | | in range |
| | | tested | | tested |
| Control 1 | No binding | No binding | No binding | No binding |
| | in range | in range | in range | in range |
| | tested | tested | tested | tested |

The functional consequence of binding to Fcγreceptors is antibody driven cellular-cytotoxicity (ADCC). C5 is a secreted protein. HepG2 cells, which are of hepatic origin and are known to produce and secrete C5 were used as the target cells to examine cell killing in ADCC assays. Results for both luciferase reporter-construct assays as well as LDH assays for cell death were negative (data not shown). Briefly, 293-T and HepG2 cells were incubated with Antibody A and Antibody B. Two positive controls were used: H929 cells were incubated with an anti-BCMA antibody that is known to cause ADCC activity in those cells, and Wil2S cells were incubated with an antiCD20 antibody that is known to cause ADCC activity in those cells.

Engineered cells with luciferase reporters (Promega, Catalog #G7010) that are activated downstream of CD16 binding were used to examine antibody binding to target cells and demonstrated to have negligible binding and reporter activation. In comparison, both the anti-CD20 mAb and the anti-BCMA antibody showed strong reporter expression. Similarly, LDH release as a marker of cell killing was measured when 293-T and HepG2 cells incubated with antibody were co-cultured with fresh human PBMCs. LDH levels in media (as a marker of ADCC activation and downstream of cell killing) were found to be similarly negligible.

Thus, engineered anti-C5 antibodies of the present invention exhibit reduced effector functions.

### Example 8

### Engineered anti-C5 antibodies mediate depletion of C5 antigen in mice

The present Example demonstrates that engineered anti-C5 antibodies of the present invention enhance depletion of C5 antigen in mice. The results described in this Example suggest that engineered anti-C5 antibodies of the present invention mediate active internalization of C5 antigen and efficiently unload them in the endosomes, consistent with these antibodies' observed enhanced pH-differential binding described in Examples 4 and 5.

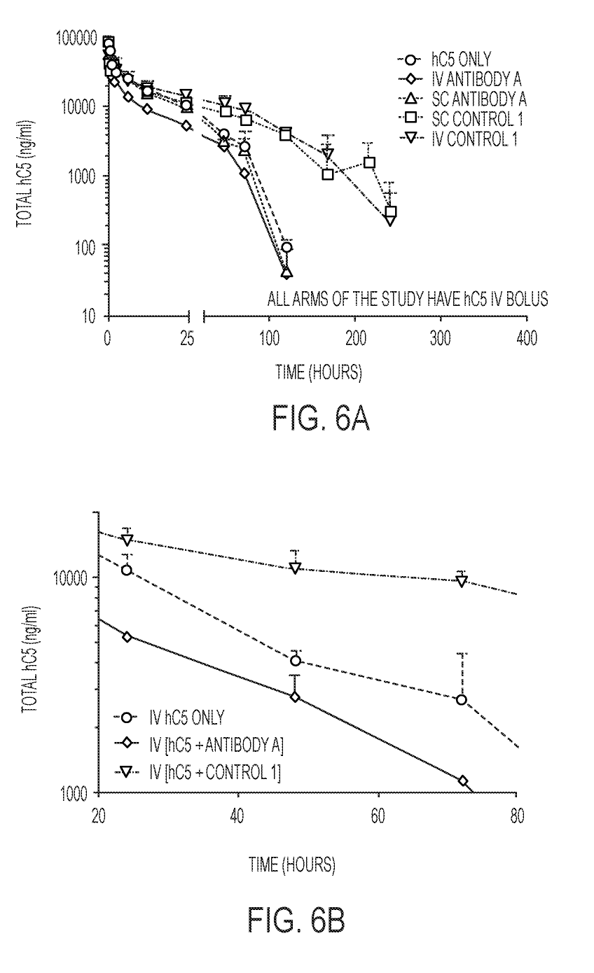

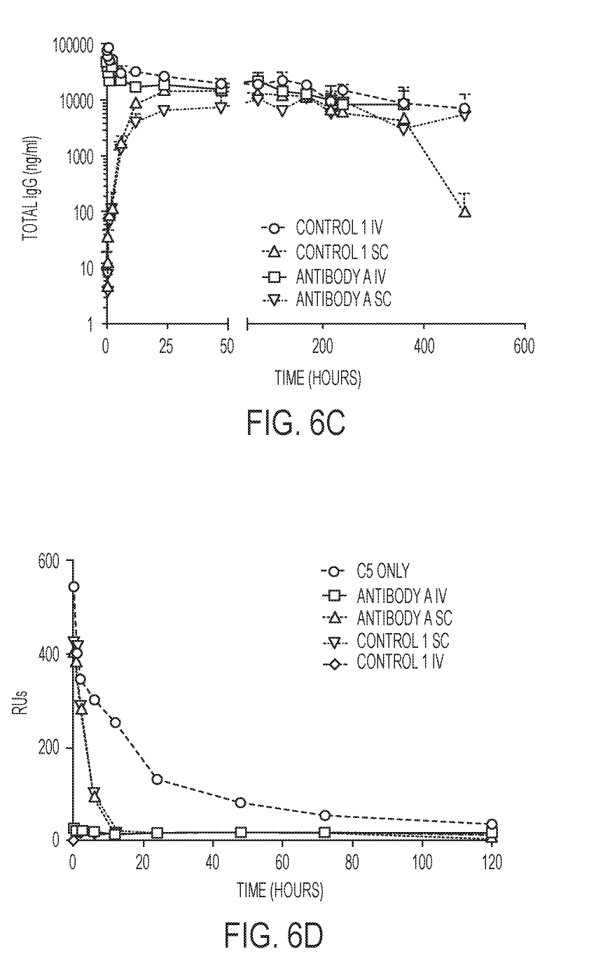

Representative results of an antigen-depletion assay are shown in FIG. 6A. CD-1 mice were injected with an intravenous bolus of hC5 alone (circles), an intravenous bolus of hC5 co-administered either with an intravenous bolus of Antibody A (diamonds) or Control 1(downward triangles), or an intravenous bolus of hC5 and a subcutaneous injections of Antibody A (upward triangles) or Control 1 (squares). The dose of administered antibodies was 2.5 mg/kg. Given the ratio of molecular weights and two binding sites for C5 per molecule of mAb, this dose amounts to slight excess (˜1.6 fold) of antigen binding sites. hC5 levels in sera were measured in all groups up to the 240-hour time point. Any data points below the lower limit of quantitation, e.g., during the latter part of the time-course, were not included in the graph in FIG. 6A.

As shown in FIG. 6A, total hC5 levels in mice co-injected with Antibody A, whether subcutaneously or intravenously, declined more rapidly than those in mice co-injected with Control 1. Antibody A was capable of rapidly depleting hC5 from serum, with >99% depletion by 120 hours after injection.

To better illustrate the magnitude of depletion at earlier time points, FIG. 6B shows a subplot of the graph in FIG. 6A, in which the axis is restricted to the 20 to 80 hour timepoint, and the y-axis is restricted to 1000 to 11,000 ng/mL. At the 72-hour time-point, total C5 levels in mice treated intravenously with Antibody A (diamonds) were approximately 90% lower than in mice treated with Control 1 antibody (downward triangles). In contrast, animals treated with Control 1 antibody exhibited a more gradual decrease of hC5, exhibiting a comparable (90%) reduction only by 168 hours. These results with the Control 1 antibody are consistent with a build-up of antibody-antigen complexes, which is common for high affinity mAbs that do not exhibit pH-differential binding.

The differences between Antibody A and Control 1 antibody could not be accounted for by changes in IgG levels, as IgG levels were comparable for all treatment arms (FIG. 6C). Measurements of free hC5 levels using a semi-quantitative Biacore® assay showed that in all treatment arms, all hC5 present in serum was bound with a mAb (FIG. 6D), indicating that the antibodies sequestered serum C5 until C5 levels were depleted.

These results indicate that engineered anti-C5 antibodies of the invention mediate depletion of C5, and do so more rapidly than do non-engineered anti-C5 antibodies. Furthermore, engineered anti-C5 antibodies of the invention mediate greater C5 antigen depletion than is observed when antigen alone is injected into mice, suggesting that engineered anti-C5 antibodies mediate active internalization of C5 antigen.

### Example 9

### Sustained Antigen Depletion by Engineered Anti-C5 Antibodies

The present Example demonstrates that engineered anti-C5 antibodies of the present invention are capable of sustained antigen depletion in mice having sustained release of C5.

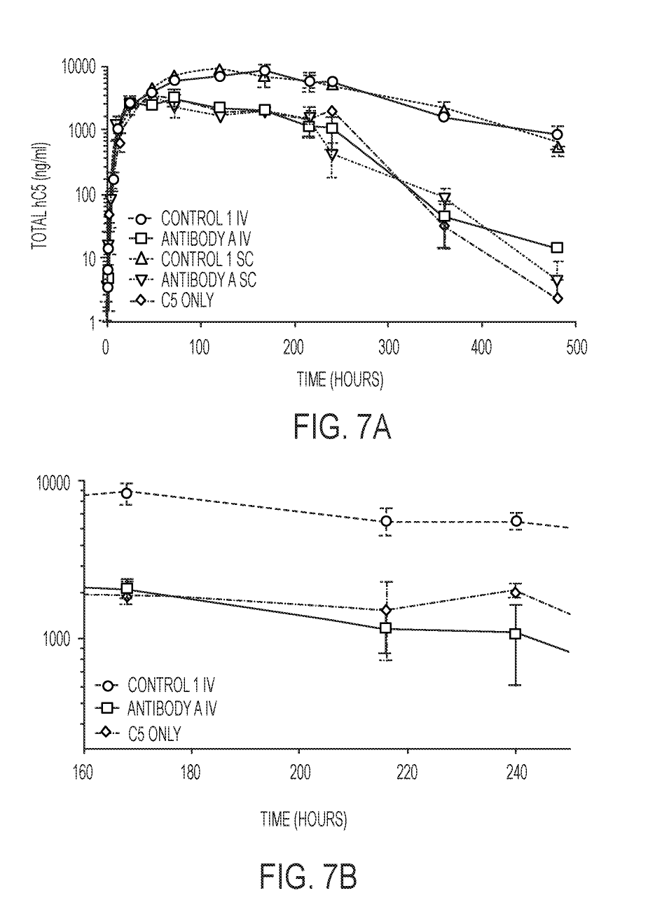

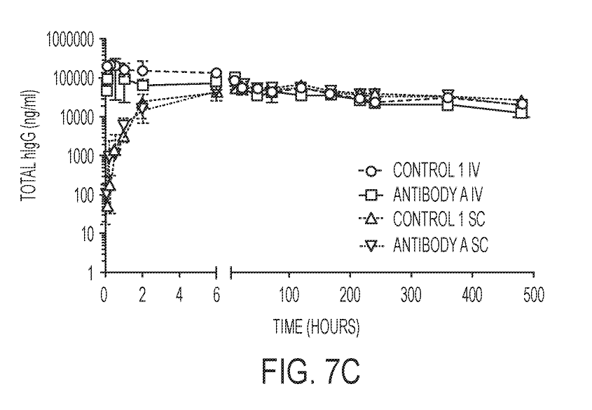

To assess the impact of engineered antibodies on steady-state levels of C5, a series of experiments were performed with severe combined immunodeficiency (SCID) mice implanted with an Alzet pump releasing human C5 (hC5) at a constant rate over the course of approximately one week. Antibody A was injected intravenously or subcutaneously at a dose of 200 μg 24 hours after pump implantation. Mice injected with Control 1 antibodies (also at a dose of 200 μg) were used as a comparison, and mice receiving C5 only were used as control. Total C5 levels in serum and free IgG (anti-C5 levels) were measured over time after injection with antibodies. The constant efflux from the mini-pump caused hC5 levels to initially rise (Tmax was approximately 24 to 48 hours established in multiple repeat studies (FIG. 7A; see also subplot in FIG. 7B)), after which steady-state levels were maintained for the patency of the pump (typically approximately 200 to 220 hours; note downward inflection point in total hC5 traces at approximately 220 hours in FIG. 7A).

Steady-state levels of hC5 were lower in mice treated with Antibody A than in mice treated with Control 1 (FIGS. 7A and 7B). Without wishing to be bound by any particular theory, the increased levels of hC5 in Control 1-treated mice appear to be the result of stabilization of hC5 that is bound to Control 1 antibodies. In contrast, the lack of stabilization of hC5 in Antibody A-treated mice appears to be due to internalization of hC5 via FcRn. Free hC5 was undetectable in serum from Antibody A-treated mice. After hC5 influx was exhausted, depletion of remaining hC5 was rapid for mice administered Antibody A, whether administered intravenously or subcutaneously, and was comparable to the depletion observed in the intravenous bolus experiments described above (>90% depletion within 100 hours). After hC5 influx was exhausted, depletion of hC5 in the mice treated with Control 1 occurred at a more gradual rate and resembled the intravenous bolus experiments.

These data demonstrate that engineered antibodies of the present invention are capable of sustained depletion of C5.

### Example 10

### Bioavailability of Intravenously or Subcutaneously Administered Engineered Anti-C5 Antibodies

The present Example demonstrates successful delivery of engineered anti-C5 antibodies of the present invention by two different routes of administration. Furthermore, the present Example demonstrates that the antibodies delivered either intravenously or subcutaneously exhibited good bioavailability in mice.

As discussed in Example 8, CD-1 mice were injected intravenously or subcutaneously with 2.5 mg/kg Antibody A or Control 1 antibody. FIG. 6C shows serum IgG levels over time in CD-1 mice.

As discussed in Example 9, SCID mice were injected intravenously or subcutaneously with Antibody A or Control 1. FIG. 7C shows serum IgG levels over time in SCID mice.

As seen in both FIG. 6C and FIG. 7C, there were modest differences in initial transient levels in mice administered Antibody A subcutaneously compared to mice administered Antibody B subcutaneously (split-axis, left in both figures), but terminal phase mAb concentrations were very similar and high in both sets of mice (split-axis, right in both figures).

### Example 11

### Cross-Species Reactivity of Engineered Antibodies

The present Example demonstrates that engineered antibodies of the present invention show cross-reactivity to C5 from species other than humans. In particular, engineered antibodies show good pH-dependent binding kinetics in cynomolgus monkeys. Thus, it is possible to conduct valid studies (such as toxicology studies) in cynomolgus monkeys using engineered antibodies of the present invention (that is, without having to develop surrogate antibodies for such studies).

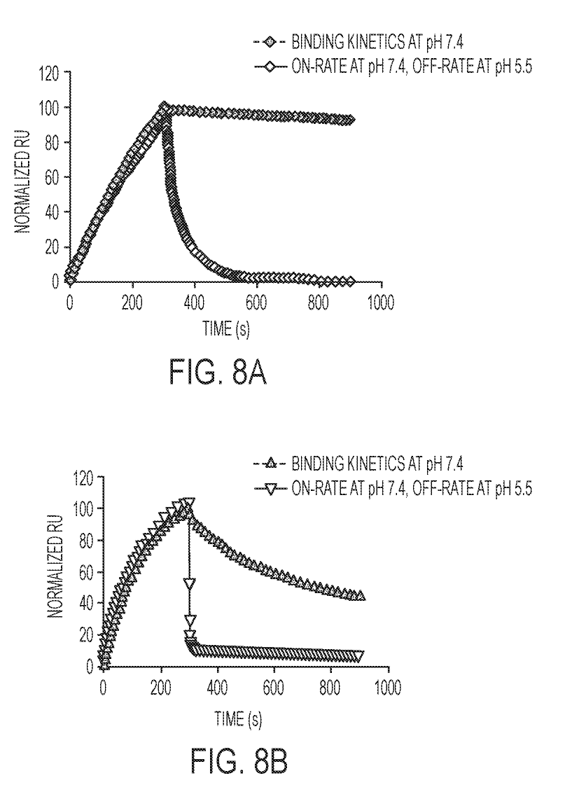

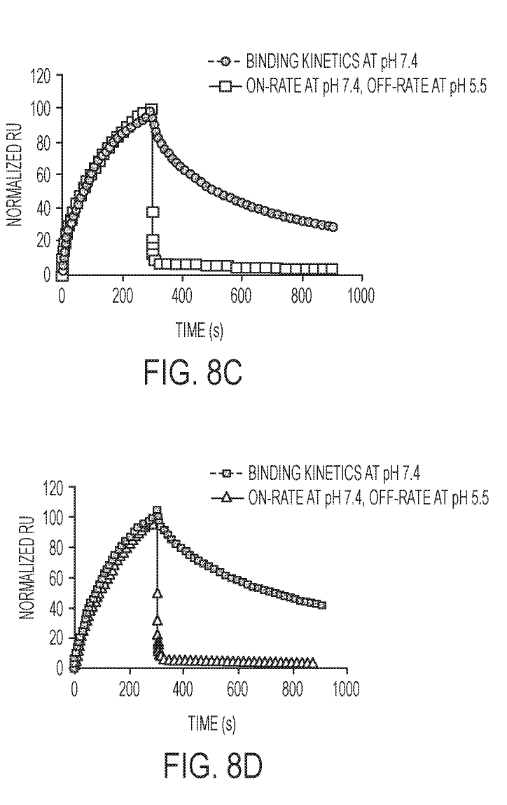

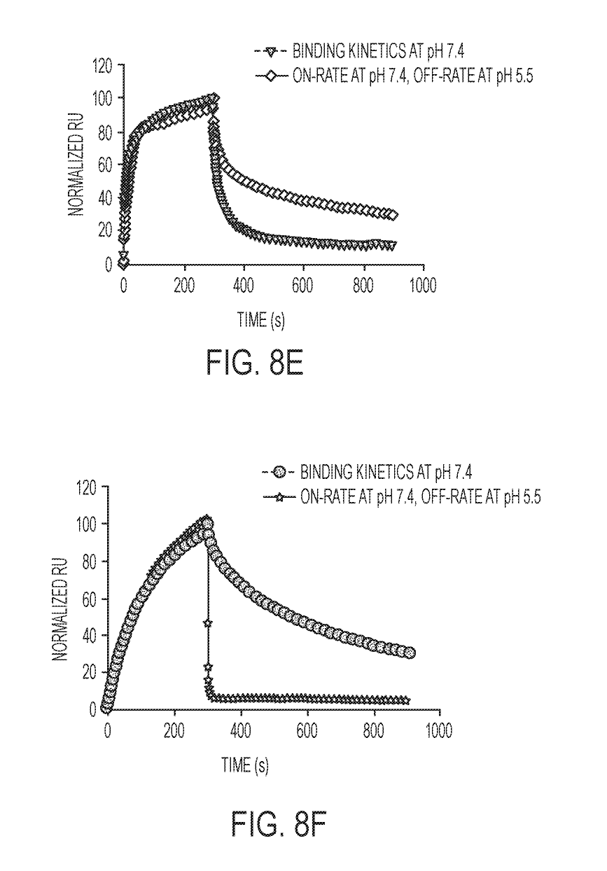

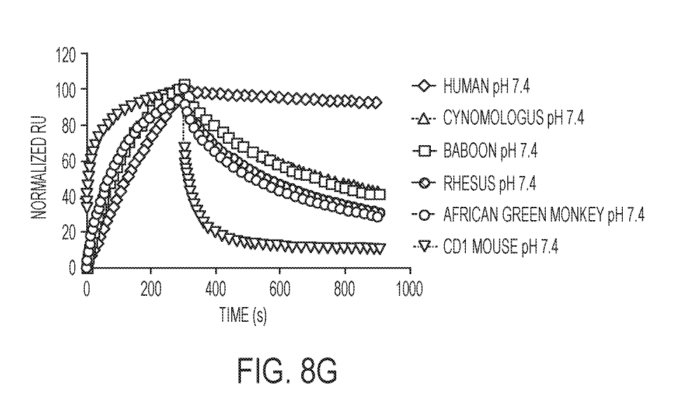

Biacore® binding assays were performed with antibody captured on the chip and sera from 16 different mammalian species as the analyte that is flowed over the chip. Sera from multiple species were identified as having specific binding with affinities in the vicinity of hC5 binding. Sera from other species tested did not exhibit binding. (Data not shown for those species.) FIGS. 8A, 8B, 8C, 8D, 8E, and 8F depict data from a representative experiment showing binding of Antibody B to C5 in serum from human, cynomolgus monkey, African Green monkey, baboon, CD1 mice, and rhesus monkey, respectively. In FIGS. 8A-8F, binding kinetics at pH 7.4 as well as on-rate at pH 7.4 and off-rate at pH 5.5 are shown.

To validate the binding as specific to C5, pull-down experiments were performed on sera from cynomolgus monkeys. Size exclusion chromatography was used to purify the resulting cynomolgus C5, which was also used to reconfirm binding on biacore. FIG. 8G depicts an overlay of binding curves and mean affinities for top non-human primate species and CD-1 mice. The non-human primate species with closest comparable binding to human was cynomolgus monkey (with a 5.7-fold lower affinity).

Table 10 depicts the estimated binding affinities of Antibody B for C5 in sera from various mammalian species at pH 7.4.

TABLE 10
Estimated binding affinities at pH 7.4

K_{d} (nM)

| | Antibody B | Control 1 |
|---|---|---|
| Purified C5 | 0.34 ± 0.14 | 0.13 ± 0.08 |
| Human serum | 0.33 ± 0.11 | 0.13 ± 0.05 |
| Cynomolgus serum | 1.9 ± 0.23 | 15.4 ± 5.7 |
| Baboon serum^{a} | 2.6 ± 0.58 | 16.25 ± 2.85 |
| Rhesus serum^{a} | 3.19 ± 0.47 | 27.82 ± 11 |
| African Green monkey | 3.2 ± 0.53 | 27.53 ± 3.4 |
| serum^{a} | | |
| CD-1 mouse serum^{a} | 5.3 ± 0.5 | NDB^{a} |

^{a}Because of the lack of purified C5 for some mammalian species, some Biacore ® affinity measurements may not be precise. Affinities were estimated assuming titers among all these species were similar to those measured in humans and cynomolgus monkey (~75 μg/mL).

In addition, cross-reactivity of Antibody A to C5 from cynomolgus monkey serum was demonstrated in pull-down experiments. The mean affinities for Antibody A, Antibody B, and Control 1 for cC5 were calculated to be 1.65, 1.9, and 15.4 nM, respectively.

Together with results from pharmacokinetic/pharmacodynamics studies and C5 depletion studies discussed below in Examples 12 and 13, respectively, the results suggest that the cynomolgus monkey can be used as a species in which to perform studies, such as toxicological studies, with engineered antibodies of the present invention.

### Example 12

### Pharmacokinetics of Engineered Antibodies in Cynomolgus Monkeys

Experiments were carried out in cynomolgus monkeys to examine the impact of multiple doses of Antibody A, Antibody B, and Control 1 on endogenous cynomolgus C5 (cC5) levels over extended periods, as well as the in vivo half-lives of these antibodies.

A dose escalation of Antibody A and Antibody B from 1 to 30 mg/kg resulted in linear increases in certain pharmacokinetic parameters. The impact of the additional H433K and N434F mutations present in Antibody B (compared to Antibody A) was evident as an improvement in the half-life of the antibody at an equivalent dose. (See Table 11, which summarizes pharmacokinetics of antibodies in cynomolgus monkeys.)

TABLE 11
Summary of pharmacokinetics observed in cynomolgus monkeys

| | | Dosage | T½ | C0/Cmax | Tmax |
|---|---|---|---|---|---|
| Compound | ROA | (mg/kg) | (hr) | (μg/mL) | (hr) |
| Antibody B | IV | 1 | 466 ± 180 | 31.3 ± 3.2 | — |
| | IV | 10 | 386 ± 17 | 338.9 ± 67.9 | — |
| | IV | 30 | 447 ± 22 | 1015.2 ± 146.9 | — |
| | SC | 30 | 192 ± 145 | 230.7 ± 30.9 | 80 ± 28 |
| Antibody A | IV | 1 | 308 ± 21 | 35.1 ± 2.8 | — |
| | IV | 10 | 325 ± 44 | 227.7 ± 19.2 | — |
| | IV | 30 | 305 ± 82 | 781.1 ± 93.1 | — |
| | SC | 30 | 258 ± 38 | 441.5 ± 36.8 | 80 ± 28 |
| Control 1 | IV | 30 | 373 ± 86 | 712.1 ± 55.4 | — |

| | AUC0-∞ | CL | Vss | F |
|---|---|---|---|---|
| Compound | (hr*μg/mL) | (mL/hr/kg) | (mL/kg) | (%) |
| Antibody B | 11558 ± 4588 | 0.10 ± 0.04 | 56 ± 6 | |
| | 91148 ± 5036 | 0.11 ± 0.01 | 60 ± 2 | — |
| | 278242 ± 21988 | 0.11 ± 0.01 | 66 ± 8 | — |
| | 115928 ± 39400 | — | — | 42 |
| Antibody A | 5410 ± 724 | 0.19 ± 0.03 | 79 ± 13 | — |
| | 67652 ± 9936 | 0.15 ± 0.02 | 68 ± 3 | — |
| | 171802 ± 46720 | 0.18 ± 0.05 | 75 ± 7 | — |
| | 189204 ± 29024 | — | — | 110 |
| Control 1 | 234243 ± 25029 | 0.13 ± 0.01 | 65 ± 10 | — |

AUC0-∞ = area under the concentration versus time curve from 0 to infinity;
CL = clearance;
F(%) = bioavailability;
IV = intravenous;
ROA = route of administration;
SC = subcutaneous;
T½ = half life;
Vss = volume of distribution at steady state

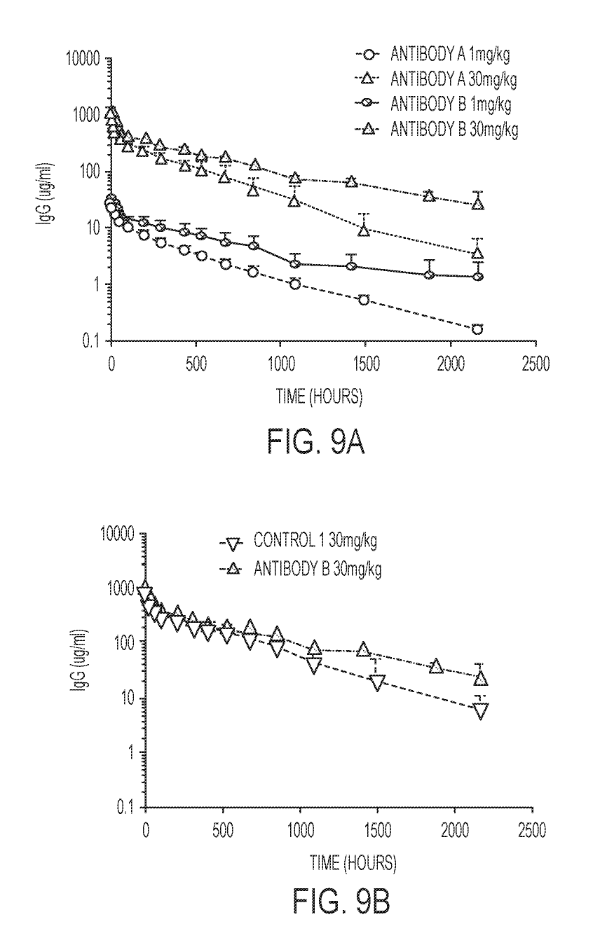

FIG. 9A shows serum IgG levels over time in monkeys intravenously administered Antibody A or Antibody B at doses of 1 or 30 mg/kg. As shown in FIG. 9A, there was a clear separation over time between Antibody A and Antibody B. This separation was observed at all three doses (1 mg/kg, 10 mg/kg (data not shown), and 30 mg/kg).

FIG. 9B shows the serum IgG levels in mice administered 30 mg/kg Antibody B or Control 1, which was comparable in the two groups until about 900+hours.

Other pharmacokinetic and pharmacodynamic parameters of Antibody A and Antibody B were evaluated in cynomolgus monkeys and are also summarized in Table 11.

### Absorption

T. occurred at 80±28 hours post-dose following a single dose of 30 mg/kg subcutaneous administration for both Antibody A and Antibody B. Subcutaneous bioavailability was 110% for Antibody A and 42% for Antibody B when compared with exposure following a single dose of 30 mg/kg administered intravenously. Compared with their half-lives after intravenous administration, the half-life of Antibody A after subcutaneous administration was comparable, while the half-life of Antibody B after subcutaneous administration was shorter (p<0.05, unpaired t-test).

### Distribution

The volume of distribution at steady state (Vₛₛ) was approximately 74 mL/kg (range from 68 to 79 mL/kg) and 60 mL/kg (range from 56 to 66 mL/kg) for Antibody A and Antibody B, respectively, across a dose range of 1 to 30 mg/kg administered intravenously (Table 11). Vₛₛ was 65±10 mL/kg for Control 1 at 30 mg/kg administered intravenously.

The distributions of the anti-C5 mAbs evaluated exhibited typical mAb biodistribution profiles, which have limited tissue distribution outside of central compartment. There was no consistent trend of change in volume of distribution with increase in dose.

### Metabolism (Clearance)

The serum clearance was estimated to be 0.17 mL/hr/kg, 0.11 mL/hr/kg, and 0.13 mL/hr/kg in cynomolgus monkeys for Antibody A, Antibody B, and Control 1 (Table 11), respectively. Linear clearance was observed for both Antibody A and Antibody B.

### Example 13

### Engineered Anti-C5 Antibodies Mediate Depletion of C5 Antigen in Cynomolgus Monkeys

The present Example further demonstrates the ability of engineered antibodies of the present invention to deplete C5 from serum via mechanisms involving pH-differential binding.

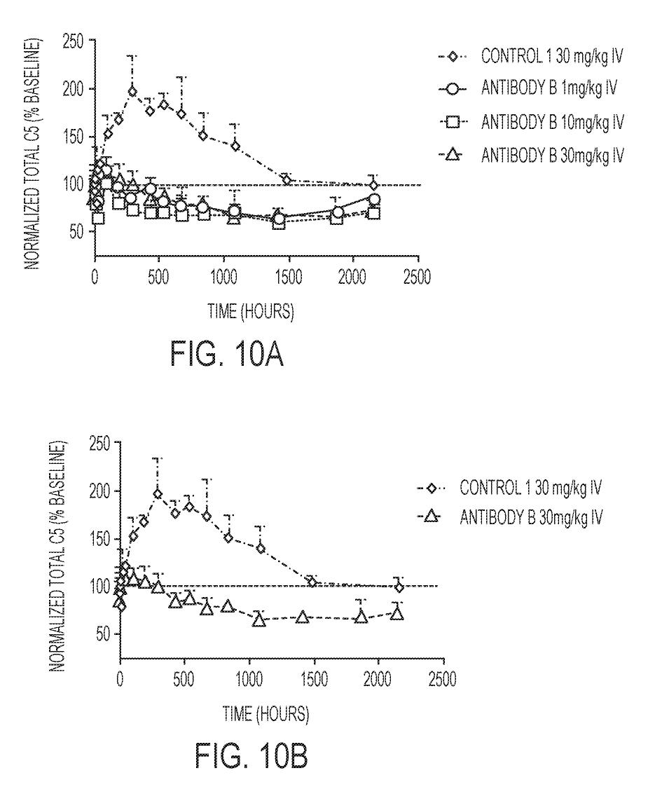

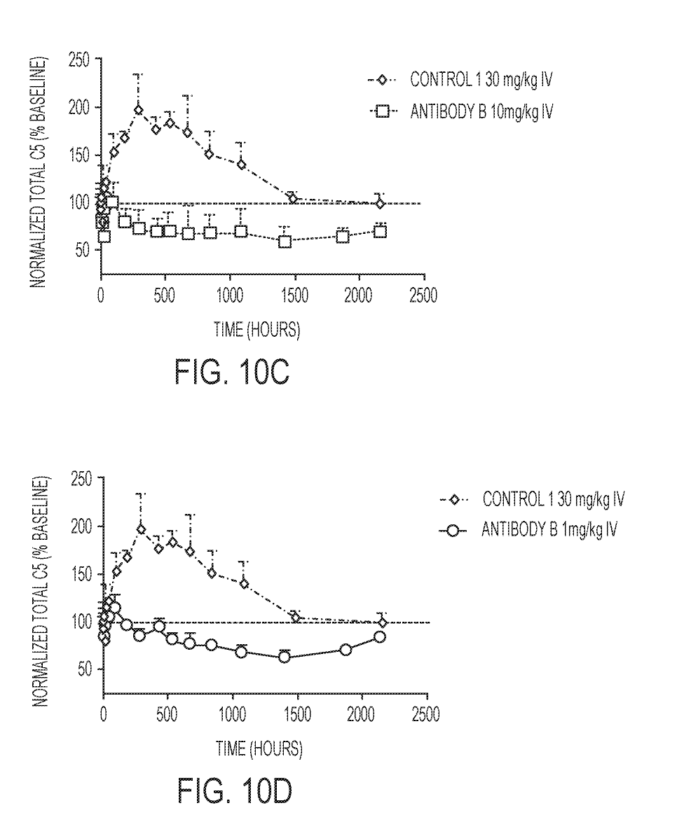

To assess the impact of engineered antibodies of the present invention on depletion of cynomolgus C5 (cC5) from serum, total cC5 levels were measured. Total cC5 levels were normalized to the baseline level for each animal and summary statistics examined for the normalized data. A comparison of normalized total cC5 levels showed that all intravenous doses of Antibody B depleted cC5 levels to below baseline, while 30 mg/kg of intravenously administered Control 1 significantly elevated total cC5 levels over the time course of the experiment. (See FIGS. 10A-10D. FIGS. 10B, 10C, and 10D are pairwise comparisons of traces in FIG. 10A.)

These differences could not be accounted for by differences in IgG levels, given the comparability of serum IgG levels in mice observed in the experiments discussed in Example 12. (See FIG. 9B).

As was the case in mice, in cynomolgus monkeys, Control 1 likely protects C5 from its normal degradation pathway, resulting in a build-up of C5:Control 1 complexes. The affinity of Control 1 for cC5 was 15.4 nM, while Antibody B displayed significantly better binding to cC5 (KD: 1.9 nM). Despite the 8-fold improvement in affinity, the lack of complex build-up in Antibody B-treated cynomolgus monkeys was consistent with a mechanism of depletion involving pH-differential binding.

### Example 14

### Safety and Toxicology Assessment of Engineered Antibodies

Modulation of the complement pathway involves immune modulation. While this modulation is inhibitory, to assess the risk that engineered antibodies may agonize an immune response and elicit cytokine responses, two independent measurements of cytokine release potential were conducted.

The ProStorm Cytokine Release Assay from ProImmune was used to test for cytokine release. Antibody B and two control mAbs (Erbitux, Campath) were co-incubated with whole blood from eight healthy human donors. Measurements of cytokines released into serum were made over a time course. In summary, the positive control Campath elicited strong cytokine responses (IL-6, IL-8, IFN-g), Erbitux elicited only responses for one cytokine (IL-2), while Antibody B did not elicit any cytokine responses.

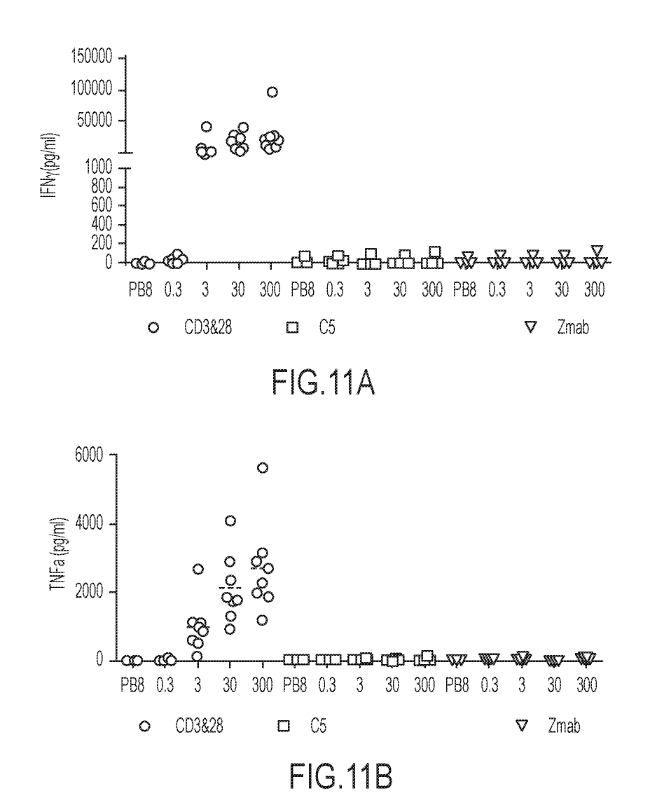

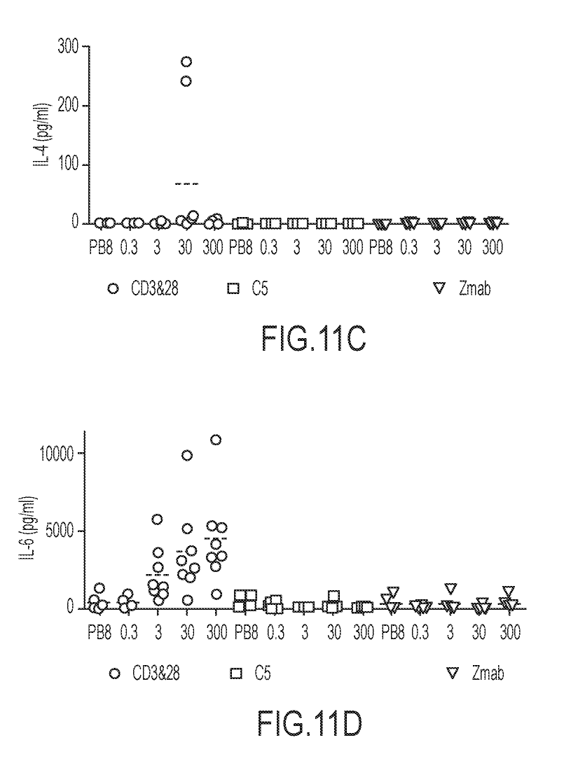

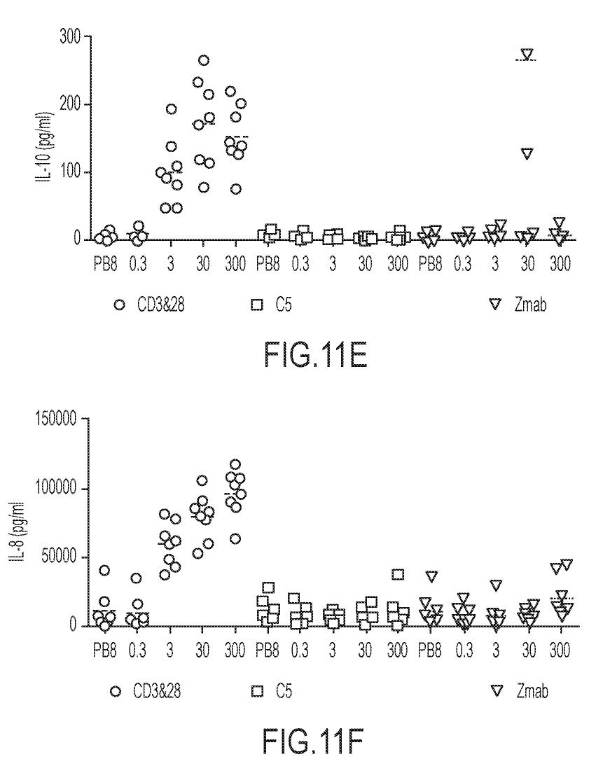

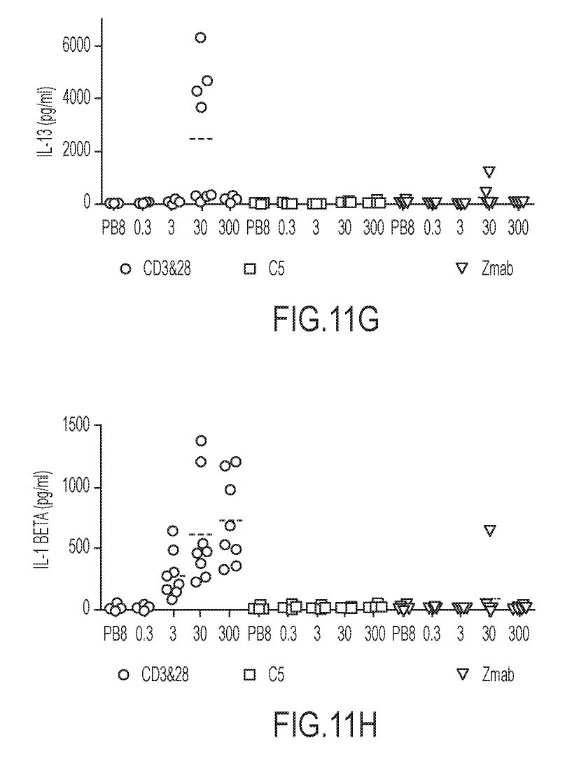

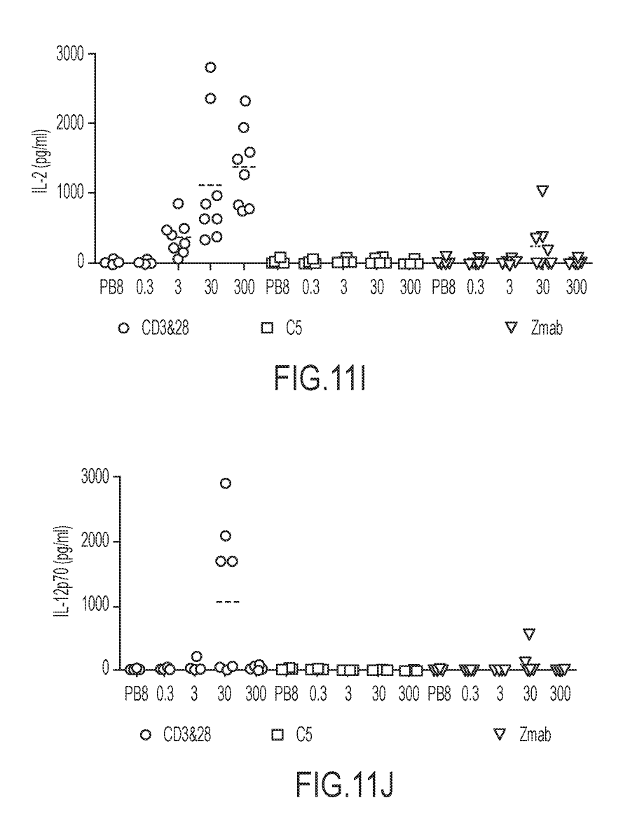

In another assay, fresh peripheral blood monocytes (PBMCs) were isolated from eight different human donors and treated with five different concentrations of Antibody B, ZmAb (a non-binding negative control), or CD3 and CD28 mAbs (positive controls). Measurements of cytokine responses were made at 24 and 48 hours. FIGS. 11A-11J show the resulting cytokine secretion levels. In summary, no significant release of IFNγ, TNFa, IL-2, IL-4, IL-6, IL-8, IL-10, IL-lb, IL-13, or IL-12p70 were observed for Antibody B. Trace amounts of IL-8 were observed in response to Antibody B (squares), but the levels were not significantly different from those of the negative control Zmab (downward triangles), which was expected to exhibit no binding to these cells. The CD3 and CD28 mAbs (circles) elicited strong secretion of all cytokines tested.

To further assess the safety of using engineered antibodies, toxicology studies were performed. To examine possible mechanism-related adverse events, a study in cynomolgus monkeys was conducted. Two out of three cynomolgus monkeys receiving 30 mg/kg of Antibody A and one out of three cynomolgus monkeys receiving 30 mg/kg of Antibody B displayed reddish-pink blisters on the skin. Monkeys were evaluated and were documented by study veterinarians to resemble immune compromise. Symptoms resolved by themselves over time. Monkeys were examined for clinical pathology parameters at the time of the rash and were reported to be normal. It is noteworthy that a documented symptom of Neisseria infections is a red-purple skin rash (http://www.cdc.gov/meningococcal/about/symptoms.html).

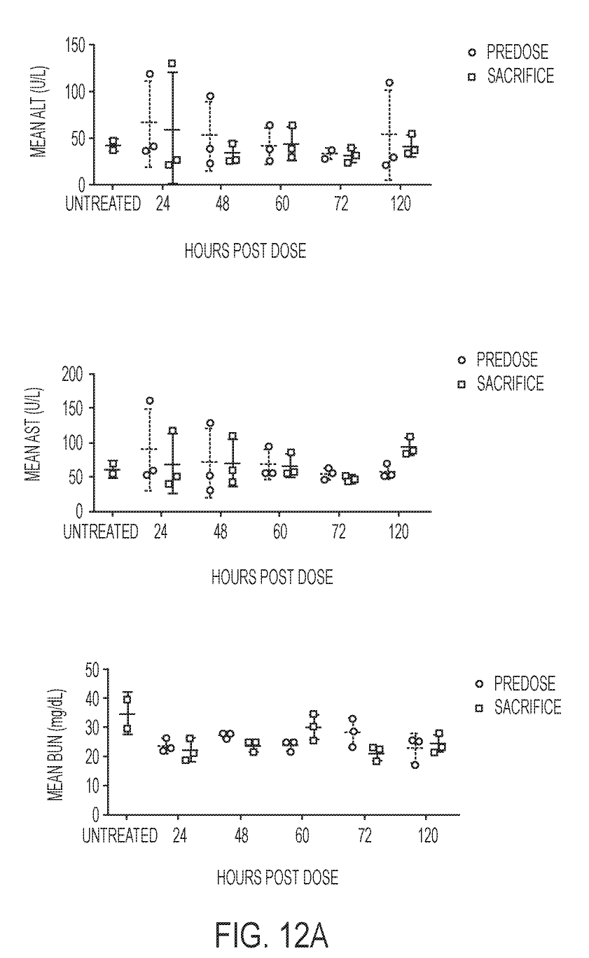

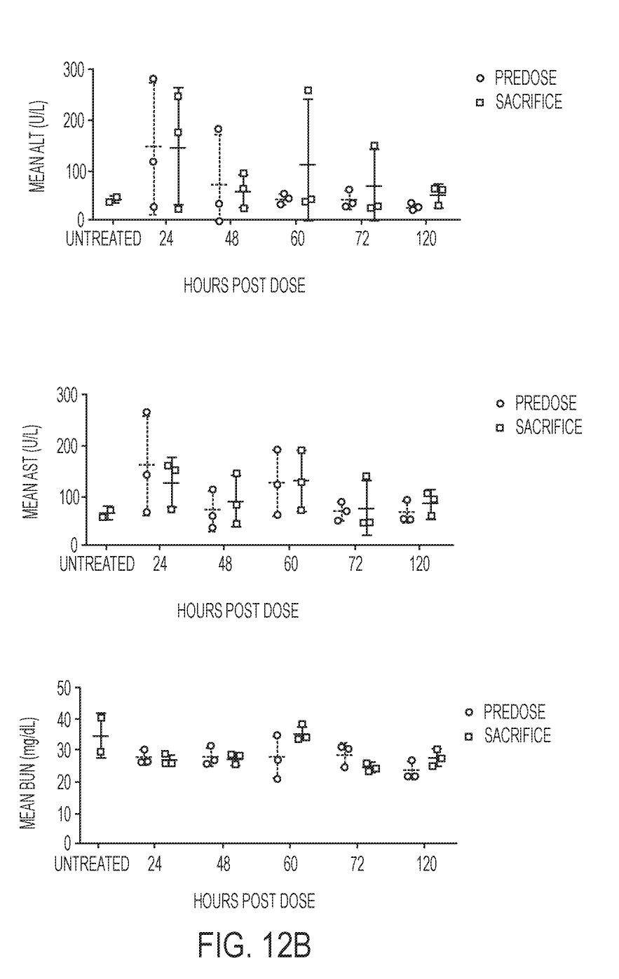

A toxicology study was also conducted in CD-1 mice. Mice were given a single intravenous administration of Antibody A or Control 2 at 10 mg/kg. Samples were collected up to 5 days post-dose and evaluated for serum chemistry endpoints alanine amino transferase (ALT), aspartate amino transferase (AST), and blood urea nitrogen (BUN). As shown in FIGS. 12A (Antibody A) and 12B (Control 2), there were no differences from pre-dose levels and post-dose levels in Antibody A-treated mice, nor were there any differences between Antibody A-treated and control mice in these parameters.

These results, together with the data from the ADCC assays discussed in Example 7, indicate that the safety and toxicology profiles of Antibody A and Antibody B appear acceptable based on studies conducted thus far.

### Example 15

### Comparison of pH-Differential Binding of Engineered Anti-C5 Antibodies

The present Example demonstrates the superior pH-differential binding properties of inventive antibodies over an engineered antibody of the prior art.

BNJ441 is an engineered antibody described in U.S. Pat. No. 9,107,861. The light chain of BNJ441 has the amino acid sequence of SEQ ID NO: 2, and the heavy chain of BNJ441 has the amino acid sequence of SEQ ID NO: 1 with the following substitutions Y27H, S57H, M429L, and N435S. BNJ441 is of the IgG 2-4 subtype.

In the present Example, the binding kinetics of BNJ441 to purified human C5 (hC5) or hC5 in human serum were compared against that of Antibody B using the same assay conditions. Binding of hC5 to immobilized mAbs (via capture with anti-Fc antibodies) were measured by Biacore® assays at 7.4 and pH 5.5.

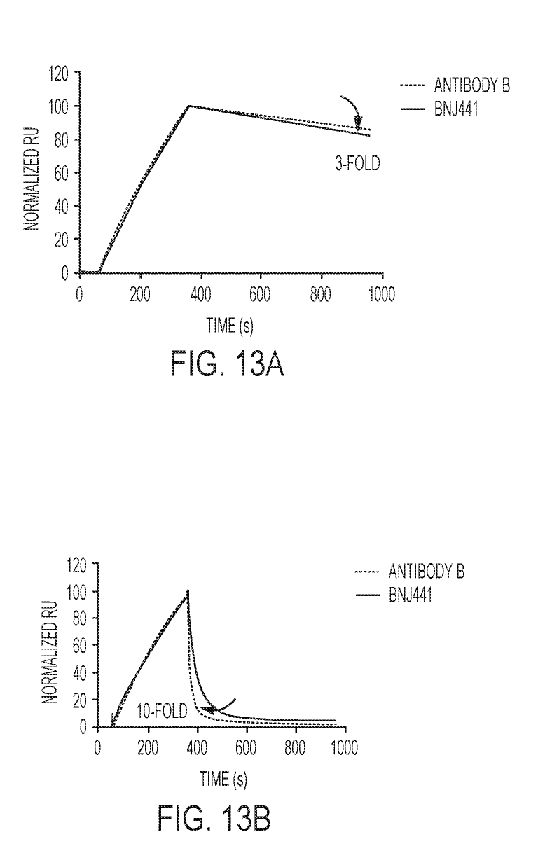

FIGS. 13A and 13B show representative binding curves over time, with the association phase at pH 7.4 and the dissociation phase at pH 7.4 (FIG. 13A) or at pH 5.5 (FIG. 13B) for Antibody B and for BNJ441 to purified hC5.

As shown in FIGS. 13A and 13B, Antibody B showed a three-fold greater affinity for purified hC5 at pH 7.4 than did BNJ441, while demonstrating a ten-fold lower affinity for hC5 at pH 5.5. Therefore, Antibody B had a greatly enhanced pH-differential binding as compared to BNJ441.

TABLE 12
Exemplary Sequences of Engineered Antibodies

SEQ ID NO: 1
QVQLVQSGAE VKKPGASVKV SCKASGYIFS NYWIQWVRQA PGQGLEWMGE ILPGSGSTEY
TENFKDRVTM TRDTSTSTVY MELSSLRSED TAVYYCARYF FGSSPNWYFD VWGQGTLVTV
SSASTKGPSV FPLAPCSRST SESTAALGCL VKDYFPEPVT VSWNSGALTS GVHTFPAVLQ
SSGLYSLSSV VTVPSSNFGT QTYTCNVDHK PSNTKVDKTV ERKCCVECPP CPAPPVAGPS
VFLFPPKPKD TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGK
SEQ ID NO: 2
DIQMTQSPSS LSASVGDRVT ITCGASENIY GALNWYQQKP GKAPKLLIYG ATNLADGVPS
RFSGSGSGTD FTLTISSLQP EDFATYYCQN VLNTPLTFGQ GTKVEIKRTV AAPSVFIFPP
SDEQLKSGTA SVVCLLNNFY PREAKVQWKV DNALQSGNSQ ESVFEQDSKD STYSLSSTLT
LSKADYEKHK VYACEVTHQG LSSPVTKSFN RGEC
SEQ ID NO: 3
QVQLVQSGAEVKKPGASVKVSCKASGYIFS
SEQ ID NO: 4
QVQLVQSGAEVKKPGASVKVSCKASGHIFS
SEQ ID NO: 5
QVQLVQSGAEVKKPGASVKVSCKASGYTFS
SEQ ID NO: 6
QVQLVQSGAEVKKPGASVKVSCKASGHTFS
SEQ ID NO: 7
NYWIQ
SEQ ID NO: 8
HYWIQ
SEQ ID NO: 9
NYWHQ
SEQ ID NO: 10
HYWHQ
SEQ ID NO: 11
WVRQAPGQGLEWMG
SEQ ID NO: 12
EILPGSGSTEYTENFKD
SEQ ID NO: 13
EIHPGSGSTEYTENFKD
SEQ ID NO: 14
EILPGSGHTEYTENFKD
SEQ ID NO: 15
EIHPGSGHTEYTENFKD
SEQ ID NO: 16
RVTMTRDTSTSTVYMELSSLRSEDTAVYYCAR
SEQ ID NO: 17
RVTMTRDTSISTAYMELSSLRSEDTAVYYCAR
SEQ ID NO: 18
YFFGSSPNWYFDV
SEQ ID NO: 19
YHFGSSPNWYFDV
SEQ ID NO: 20
WGQGTLVTVSS
SEQ ID NO: 21
QVQLVQSGAEVKKPGASVKVSCKASGYIFS NYWIQ WVRQAPGQGLEWMG EILPGSGSTEYTENFKD
RVTMTRDTSTSTVYMELSSLRSEDTAVYYCAR YFFGSSPNWYFDV WGQGTLVTVSS
SEQ ID NO: 22
QVQLVQSGAEVKKPGASVKVSCKASGYIFS HYWIQ WVRQAPGQGLEWMG EILPGSGSTEYTENFKD
RVTMTRDTSTSTVYMELSSLRSEDTAVYYCAR YFFGSSPNWYFDV WGQGTLVTVSS
SEQ ID NO: 23
QVQLVQSGAEVKKPGASVKVSCKASGYIFS NYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 24
QVQLVQSGAEVKKPGASVKVSCKASGYIFS HYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 25
QVQLVQSGAEVKKPGASVKVSCKASGHIFS NYWIQ WVRQAPGQGLEWMG EILPGSGSTEYTENFKD
RVTMTRDTSTSTVYMELSSLRSEDTAVYYCAR YFFGSSPNWYFDV WGQGTLVTVSS
SEQ ID NO: 26
QVQLVQSGAEVKKPGASVKVSCKASGHIFS HYWIQ WVRQAPGQGLEWMG EILPGSGSTEYTENFKD
RVTMTRDTSTSTVYMELSSLRSEDTAVYYCAR YFFGSSPNWYFDV WGQGTLVTVSS
SEQ ID NO: 27
QVQLVQSGAEVKKPGASVKVSCKASGHIFS NYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 28
QVQLVQSGAEVKKPGASVKVSCKASGHIFS HYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 29
QVQLVQSGAEVKKPGASVKVSCKASGYTFS NYWIQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 30
QVQLVQSGAEVKKPGASVKVSCKASGYTFS HYWIQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 31
QVQLVQSGAEVKKPGASVKVSCKASGYTFS NYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 32
QVQLVQSGAEVKKPGASVKVSCKASGYTFS HYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 33
QVQLVQSGAEVKKPGASVKVSCKASGHTFS NYWIQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 34
QVQLVQSGAEVKKPGASVKVSCKASGHTFS HYWIQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 35
QVQLVQSGAEVKKPGASVKVSCKASGHTFS NYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 36
QVQLVQSGAEVKKPGASVKVSCKASGHTFS HYWHQ WVRQAPGQGLEWMG
EILPGSGSTEYTENFKD RVTMTRDTSTSTVYMELSSLRSEDTAVYYCAR YFFGSSPNWYFDV
WGQGTLVTVSS
SEQ ID NO: 37
QVQLVQSGAEVKKPGASVKVSCKASGYIFS NYWIQ WVRQAPGQGLEWMG EIHPGSGSTEYTENFKD
RVTMTRDTSTSTVYMELSSLRSEDTAVYYCAR YFFGSSPNWYFDV WGQGTLVTVSS
SEQ ID NO: 38
QVQLVQSGAEVKKPGASVKVSCKASGYIFS HYWIQ WVRQAPGQGLEWMG EIHPGSGSTEYTENFKD
RVTMTRDTSTSTVYMELSSLRSEDTAVYYCAR YFFGSSPNWYFDV WGQGTLVTVSS
SEQ ID NO: 39
QVQLVQSGAEVKKPGASVKVSCKASGYIFS NYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 40
QVQLVQSGAEVKKPGASVKVSCKASGYIFS HYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 41
QVQLVQSGAEVKKPGASVKVSCKASGHIFS NYWIQ WVRQAPGQGLEWMG EIHPGSGSTEYTENFKD
RVTMTRDTSTSTVYMELSSLRSEDTAVYYCAR YFFGSSPNWYFDV WGQGTLVTVSS
SEQ ID NO: 42
QVQLVQSGAEVKKPGASVKVSCKASGHIFS HYWIQ WVRQAPGQGLEWMG EIHPGSGSTEYTENFKD
RVTMTRDTSTSTVYMELSSLRSEDTAVYYCAR YFFGSSPNWYFDV WGQGTLVTVSS
SEQ ID NO: 43
QVQLVQSGAEVKKPGASVKVSCKASGHIFS NYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 44
QVQLVQSGAEVKKPGASVKVSCKASGHIFS HYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 45
QVQLVQSGAEVKKPGASVKVSCKASGYTFS NYWIQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 46
QVQLVQSGAEVKKPGASVKVSCKASGYTFS HYWIQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 47
QVQLVQSGAEVKKPGASVKVSCKASGYTFS NYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 48
QVQLVQSGAEVKKPGASVKVSCKASGYTFS HYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 49
QVQLVQSGAEVKKPGASVKVSCKASGHTFS NYWIQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 50
QVQLVQSGAEVKKPGASVKVSCKASGHTFS HYWIQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 51
QVQLVQSGAEVKKPGASVKVSCKASGHTFS NYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 52
QVQLVQSGAEVKKPGASVKVSCKASGHTFS HYWHQ WVRQAPGQGLEWMG
EIHPGSGSTEYTENFKD RVTMTRDTSTSTVYMELSSLRSEDTAVYYCAR YFFGSSPNWYFDV
WGQGTLVTVSS
SEQ ID NO: 53
QVQLVQSGAEVKKPGASVKVSCKASGYIFS NYWIQ WVRQAPGQGLEWMG EILPGSGHTEYTENFKD
RVTMTRDTSTSTVYMELSSLRSEDTAVYYCAR YFFGSSPNWYFDV WGQGTLVTVSS
SEQ ID NO: 54
QVQLVQSGAEVKKPGASVKVSCKASGYIFS HYWIQ WVRQAPGQGLEWMG EILPGSGHTEYTENFKD
RVTMTRDTSTSTVYMELSSLRSEDTAVYYCAR YFFGSSPNWYFDV WGQGTLVTVSS
SEQ ID NO: 55
QVQLVQSGAEVKKPGASVKVSCKASGYIFS NYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 56
QVQLVQSGAEVKKPGASVKVSCKASGYIFS HYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 57
QVQLVQSGAEVKKPGASVKVSCKASGHIFS NYWIQ WVRQAPGQGLEWMG EILPGSGHTEYTENFKD
RVTMTRDTSTSTVYMELSSLRSEDTAVYYCAR YFFGSSPNWYFDV WGQGTLVTVSS
SEQ ID NO: 58
QVQLVQSGAEVKKPGASVKVSCKASGHIFS HYWIQ WVRQAPGQGLEWMG EILPGSGHTEYTENFKD
RVTMTRDTSTSTVYMELSSLRSEDTAVYYCAR YFFGSSPNWYFDV WGQGTLVTVSS
SEQ ID NO: 59
QVQLVQSGAEVKKPGASVKVSCKASGHIFS NYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 60
QVQLVQSGAEVKKPGASVKVSCKASGHIFS HYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 61
QVQLVQSGAEVKKPGASVKVSCKASGYTFS NYWIQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 62
QVQLVQSGAEVKKPGASVKVSCKASGYTFS HYWIQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 63
QVQLVQSGAEVKKPGASVKVSCKASGYTFS NYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 64
QVQLVQSGAEVKKPGASVKVSCKASGYTFS HYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 65
QVQLVQSGAEVKKPGASVKVSCKASGHTFS NYWIQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 66
QVQLVQSGAEVKKPGASVKVSCKASGHTFS HYWIQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 67
QVQLVQSGAEVKKPGASVKVSCKASGHTFS NYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 68
QVQLVQSGAEVKKPGASVKVSCKASGHTFS HYWHQ WVRQAPGQGLEWMG
EILPGSGHTEYTENFKD RVTMTRDTSTSTVYMELSSLRSEDTAVYYCAR YFFGSSPNWYFDV
WGQGTLVTVSS
SEQ ID NO: 69
QVQLVQSGAEVKKPGASVKVSCKASGYIFS NYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 70
QVQLVQSGAEVKKPGASVKVSCKASGYIFS HYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 71
QVQLVQSGAEVKKPGASVKVSCKASGYIFS NYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 72
QVQLVQSGAEVKKPGASVKVSCKASGYIFS HYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 73
QVQLVQSGAEVKKPGASVKVSCKASGHIFS NYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 74
QVQLVQSGAEVKKPGASVKVSCKASGHIFS HYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 75
QVQLVQSGAEVKKPGASVKVSCKASGHIFS NYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 76
QVQLVQSGAEVKKPGASVKVSCKASGHIFS HYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 77
QVQLVQSGAEVKKPGASVKVSCKASGYTFS NYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 78
QVQLVQSGAEVKKPGASVKVSCKASGYTFS HYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 79
QVQLVQSGAEVKKPGASVKVSCKASGYTFS NYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 80
QVQLVQSGAEVKKPGASVKVSCKASGYTFS HYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 81
QVQLVQSGAEVKKPGASVKVSCKASGHTFS NYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 82
QVQLVQSGAEVKKPGASVKVSCKASGHTFS HYWIQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 83
QVQLVQSGAEVKKPGASVKVSCKASGHTFS NYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 84
QVQLVQSGAEVKKPGASVKVSCKASGHTFS HYWHQ WVRQAPGQGLEWMG
EIHPGSGHTEYTENFKD RVTMTRDTSTSTVYMELSSLRSEDTAVYYCAR YFFGSSPNWYFDV
WGQGTLVTVSS
SEQ ID NO: 85
QVQLVQSGAEVKKPGASVKVSCKASGYIFS NYWIQ WVRQAPGQGLEWMG EILPGSGSTEYTENFKD
RVTMTRDTSISTAYMELSSLRSEDTAVYYCAR YFFGSSPNWYFDV WGQGTLVTVSS
SEQ ID NO: 86
QVQLVQSGAEVKKPGASVKVSCKASGYIFS HYWIQ WVRQAPGQGLEWMG EILPGSGSTEYTENFKD
RVTMTRDTSISTAYMELSSLRSEDTAVYYCAR YFFGSSPNWYFDV WGQGTLVTVSS
SEQ ID NO: 87
QVQLVQSGAEVKKPGASVKVSCKASGYIFS NYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 88
QVQLVQSGAEVKKPGASVKVSCKASGYIFS HYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 89
QVQLVQSGAEVKKPGASVKVSCKASGHIFS NYWIQ WVRQAPGQGLEWMG EILPGSGSTEYTENFKD
RVTMTRDTSISTAYMELSSLRSEDTAVYYCAR YFFGSSPNWYFDV WGQGTLVTVSS
SEQ ID NO: 90
QVQLVQSGAEVKKPGASVKVSCKASGHIFS HYWIQ WVRQAPGQGLEWMG EILPGSGSTEYTENFKD
RVTMTRDTSISTAYMELSSLRSEDTAVYYCAR YFFGSSPNWYFDV WGQGTLVTVSS
SEQ ID NO: 91
QVQLVQSGAEVKKPGASVKVSCKASGHIFS NYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 92
QVQLVQSGAEVKKPGASVKVSCKASGHIFS HYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 93
QVQLVQSGAEVKKPGASVKVSCKASGYTFS NYWIQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 94
QVQLVQSGAEVKKPGASVKVSCKASGYTFS HYWIQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 95
QVQLVQSGAEVKKPGASVKVSCKASGYTFS NYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 96
QVQLVQSGAEVKKPGASVKVSCKASGYTFS HYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 97
QVQLVQSGAEVKKPGASVKVSCKASGHTFS NYWIQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 98
QVQLVQSGAEVKKPGASVKVSCKASGHTFS HYWIQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 99
QVQLVQSGAEVKKPGASVKVSCKASGHTFS NYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 100
QVQLVQSGAEVKKPGASVKVSCKASGHTFS HYWHQ WVRQAPGQGLEWMG
EILPGSGSTEYTENFKD RVTMTRDTSISTAYMELSSLRSEDTAVYYCAR YFFGSSPNWYFDV
WGQGTLVTVSS
SEQ ID NO: 101
QVQLVQSGAEVKKPGASVKVSCKASGYIFS NYWIQ WVRQAPGQGLEWMG EIHPGSGSTEYTENFKD
RVTMTRDTSISTAYMELSSLRSEDTAVYYCAR YFFGSSPNWYFDV WGQGTLVTVSS
SEQ ID NO: 102
QVQLVQSGAEVKKPGASVKVSCKASGYIFS HYWIQ WVRQAPGQGLEWMG EIHPGSGSTEYTENFKD
RVTMTRDTSISTAYMELSSLRSEDTAVYYCAR YFFGSSPNWYFDV WGQGTLVTVSS
SEQ ID NO: 103
QVQLVQSGAEVKKPGASVKVSCKASGYIFS NYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 104
QVQLVQSGAEVKKPGASVKVSCKASGYIFS HYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 105
QVQLVQSGAEVKKPGASVKVSCKASGHIFS NYWIQ WVRQAPGQGLEWMG EIHPGSGSTEYTENFKD
RVTMTRDTSISTAYMELSSLRSEDTAVYYCAR YFFGSSPNWYFDV WGQGTLVTVSS
SEQ ID NO: 106
QVQLVQSGAEVKKPGASVKVSCKASGHIFS HYWIQ WVRQAPGQGLEWMG EIHPGSGSTEYTENFKD
RVTMTRDTSISTAYMELSSLRSEDTAVYYCAR YFFGSSPNWYFDV WGQGTLVTVSS
SEQ ID NO: 107
QVQLVQSGAEVKKPGASVKVSCKASGHIFS NYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 108
QVQLVQSGAEVKKPGASVKVSCKASGHIFS HYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 109
QVQLVQSGAEVKKPGASVKVSCKASGYTFS NYWIQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 110
QVQLVQSGAEVKKPGASVKVSCKASGYTFS HYWIQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 111
QVQLVQSGAEVKKPGASVKVSCKASGYTFS NYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 112
QVQLVQSGAEVKKPGASVKVSCKASGYTFS HYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 113
QVQLVQSGAEVKKPGASVKVSCKASGHTFS NYWIQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 114
QVQLVQSGAEVKKPGASVKVSCKASGHTFS HYWIQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 115
QVQLVQSGAEVKKPGASVKVSCKASGHTFS NYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 116
QVQLVQSGAEVKKPGASVKVSCKASGHTFS HYWHQ WVRQAPGQGLEWMG
EIHPGSGSTEYTENFKD RVTMTRDTSISTAYMELSSLRSEDTAVYYCAR YFFGSSPNWYFDV
WGQGTLVTVSS
SEQ ID NO: 117
QVQLVQSGAEVKKPGASVKVSCKASGYIFS NYWIQ WVRQAPGQGLEWMG EILPGSGHTEYTENFKD
RVTMTRDTSISTAYMELSSLRSEDTAVYYCAR YFFGSSPNWYFDV WGQGTLVTVSS
SEQ ID NO: 118
QVQLVQSGAEVKKPGASVKVSCKASGYIFS HYWIQ WVRQAPGQGLEWMG EILPGSGHTEYTENFKD
RVTMTRDTSISTAYMELSSLRSEDTAVYYCAR YFFGSSPNWYFDV WGQGTLVTVSS
SEQ ID NO: 119
QVQLVQSGAEVKKPGASVKVSCKASGYIFS NYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 120
QVQLVQSGAEVKKPGASVKVSCKASGYIFS HYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 121
QVQLVQSGAEVKKPGASVKVSCKASGHIFS NYWIQ WVRQAPGQGLEWMG EILPGSGHTEYTENFKD
RVTMTRDTSISTAYMELSSLRSEDTAVYYCAR YFFGSSPNWYFDV WGQGTLVTVSS
SEQ ID NO: 122
QVQLVQSGAEVKKPGASVKVSCKASGHIFS HYWIQ WVRQAPGQGLEWMG EILPGSGHTEYTENFKD
RVTMTRDTSISTAYMELSSLRSEDTAVYYCAR YFFGSSPNWYFDV WGQGTLVTVSS
SEQ ID NO: 123
QVQLVQSGAEVKKPGASVKVSCKASGHIFS NYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 124
QVQLVQSGAEVKKPGASVKVSCKASGHIFS HYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 125
QVQLVQSGAEVKKPGASVKVSCKASGYTFS NYWIQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 126
QVQLVQSGAEVKKPGASVKVSCKASGYTFS HYWIQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 127
QVQLVQSGAEVKKPGASVKVSCKASGYTFS NYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 128
QVQLVQSGAEVKKPGASVKVSCKASGYTFS HYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 129
QVQLVQSGAEVKKPGASVKVSCKASGHTFS NYWIQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 130
QVQLVQSGAEVKKPGASVKVSCKASGHTFS HYWIQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 131
QVQLVQSGAEVKKPGASVKVSCKASGHTFS NYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 132
QVQLVQSGAEVKKPGASVKVSCKASGHTFS HYWHQ WVRQAPGQGLEWMG
EILPGSGHTEYTENFKD RVTMTRDTSISTAYMELSSLRSEDTAVYYCAR YFFGSSPNWYFDV
WGQGTLVTVSS
SEQ ID NO: 133
QVQLVQSGAEVKKPGASVKVSCKASGYIFS NYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 134
QVQLVQSGAEVKKPGASVKVSCKASGYIFS HYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 135
QVQLVQSGAEVKKPGASVKVSCKASGYIFS NYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 136
QVQLVQSGAEVKKPGASVKVSCKASGYIFS HYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 137
QVQLVQSGAEVKKPGASVKVSCKASGHIFS NYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 138
QVQLVQSGAEVKKPGASVKVSCKASGHIFS HYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 139
QVQLVQSGAEVKKPGASVKVSCKASGHIFS NYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 140
QVQLVQSGAEVKKPGASVKVSCKASGHIFS HYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 141
QVQLVQSGAEVKKPGASVKVSCKASGYTFS NYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 142
QVQLVQSGAEVKKPGASVKVSCKASGYTFS HYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 143
QVQLVQSGAEVKKPGASVKVSCKASGYTFS NYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 144
QVQLVQSGAEVKKPGASVKVSCKASGYTFS HYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 145
QVQLVQSGAEVKKPGASVKVSCKASGHTFS NYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 146
QVQLVQSGAEVKKPGASVKVSCKASGHTFS HYWIQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 147
QVQLVQSGAEVKKPGASVKVSCKASGHTFS NYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 148
QVQLVQSGAEVKKPGASVKVSCKASGHTFS HYWHQ WVRQAPGQGLEWMG
EIHPGSGHTEYTENFKD RVTMTRDTSISTAYMELSSLRSEDTAVYYCAR YFFGSSPNWYFDV
WGQGTLVTVSS
SEQ ID NO: 149
QVQLVQSGAEVKKPGASVKVSCKASGYIFS NYWIQ WVRQAPGQGLEWMG EILPGSGSTEYTENFKD
RVTMTRDTSTSTVYMELSSLRSEDTAVYYCAR YHFGSSPNWYFDV WGQGTLVTVSS
SEQ ID NO: 150
QVQLVQSGAEVKKPGASVKVSCKASGYIFS HYWIQ WVRQAPGQGLEWMG EILPGSGSTEYTENFKD
RVTMTRDTSTSTVYMELSSLRSEDTAVYYCAR YHFGSSPNWYFDV WGQGTLVTVSS
SEQ ID NO: 151
QVQLVQSGAEVKKPGASVKVSCKASGYIFS NYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 152
QVQLVQSGAEVKKPGASVKVSCKASGYIFS HYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 153
QVQLVQSGAEVKKPGASVKVSCKASGHIFS NYWIQ WVRQAPGQGLEWMG EILPGSGSTEYTENFKD
RVTMTRDTSTSTVYMELSSLRSEDTAVYYCAR YHFGSSPNWYFDV WGQGTLVTVSS
SEQ ID NO: 154
QVQLVQSGAEVKKPGASVKVSCKASGHIFS HYWIQ WVRQAPGQGLEWMG EILPGSGSTEYTENFKD
RVTMTRDTSTSTVYMELSSLRSEDTAVYYCAR YHFGSSPNWYFDV WGQGTLVTVSS
SEQ ID NO: 155
QVQLVQSGAEVKKPGASVKVSCKASGHIFS NYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 156
QVQLVQSGAEVKKPGASVKVSCKASGHIFS HYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 157
QVQLVQSGAEVKKPGASVKVSCKASGYTFS NYWIQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 158
QVQLVQSGAEVKKPGASVKVSCKASGYTFS HYWIQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 159
QVQLVQSGAEVKKPGASVKVSCKASGYTFS NYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 160
QVQLVQSGAEVKKPGASVKVSCKASGYTFS HYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 161
QVQLVQSGAEVKKPGASVKVSCKASGHTFS NYWIQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 162
QVQLVQSGAEVKKPGASVKVSCKASGHTFS HYWIQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 163
QVQLVQSGAEVKKPGASVKVSCKASGHTFS NYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 164
QVQLVQSGAEVKKPGASVKVSCKASGHTFS HYWHQ WVRQAPGQGLEWMG
EILPGSGSTEYTENFKD RVTMTRDTSTSTVYMELSSLRSEDTAVYYCAR YHFGSSPNWYFDV
WGQGTLVTVSS
SEQ ID NO: 165
QVQLVQSGAEVKKPGASVKVSCKASGYIFS NYWIQ WVRQAPGQGLEWMG EIHPGSGSTEYTENFKD
RVTMTRDTSTSTVYMELSSLRSEDTAVYYCAR YHFGSSPNWYFDV WGQGTLVTVSS
SEQ ID NO: 166
QVQLVQSGAEVKKPGASVKVSCKASGYIFS HYWIQ WVRQAPGQGLEWMG EIHPGSGSTEYTENFKD
RVTMTRDTSTSTVYMELSSLRSEDTAVYYCAR YHFGSSPNWYFDV WGQGTLVTVSS
SEQ ID NO: 167
QVQLVQSGAEVKKPGASVKVSCKASGYIFS NYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 168
QVQLVQSGAEVKKPGASVKVSCKASGYIFS HYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 169
QVQLVQSGAEVKKPGASVKVSCKASGHIFS NYWIQ WVRQAPGQGLEWMG EIHPGSGSTEYTENFKD
RVTMTRDTSTSTVYMELS SLRSEDTAVYYCAR YHFGSSPNWYFDV WGQGTLVTVSS
SEQ ID NO: 170
QVQLVQSGAEVKKPGASVKVSCKASGHIFS HYWIQ WVRQAPGQGLEWMG EIHPGSGSTEYTENFKD
RVTMTRDTSTSTVYMELSSLRSEDTAVYYCAR YHFGSSPNWYFDV WGQGTLVTVSS
SEQ ID NO: 171
QVQLVQSGAEVKKPGASVKVSCKASGHIFS NYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 172
QVQLVQSGAEVKKPGASVKVSCKASGHIFS HYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 173
QVQLVQSGAEVKKPGASVKVSCKASGYTFS NYWIQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 174
QVQLVQSGAEVKKPGASVKVSCKASGYTFS HYWIQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 175
QVQLVQSGAEVKKPGASVKVSCKASGYTFS NYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 176
QVQLVQSGAEVKKPGASVKVSCKASGYTFS HYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 177
QVQLVQSGAEVKKPGASVKVSCKASGHTFS NYWIQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 178
QVQLVQSGAEVKKPGASVKVSCKASGHTFS HYWIQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 179
QVQLVQSGAEVKKPGASVKVSCKASGHTFS NYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 180
QVQLVQSGAEVKKPGASVKVSCKASGHTFS HYWHQ WVRQAPGQGLEWMG
EIHPGSGSTEYTENFKD RVTMTRDTSTSTVYMELSSLRSEDTAVYYCAR YHFGSSPNWYFDV
WGQGTLVTVSS
SEQ ID NO: 181
QVQLVQSGAEVKKPGASVKVSCKASGYIFS NYWIQ WVRQAPGQGLEWMG EILPGSGHTEYTENFKD
RVTMTRDTSTSTVYMELSSLRSEDTAVYYCAR YHFGSSPNWYFDV WGQGTLVTVSS
SEQ ID NO: 182
QVQLVQSGAEVKKPGASVKVSCKASGYIFS HYWIQ WVRQAPGQGLEWMG EILPGSGHTEYTENFKD
RVTMTRDTSTSTVYMELSSLRSEDTAVYYCAR YHFGSSPNWYFDV WGQGTLVTVSS
SEQ ID NO: 183
QVQLVQSGAEVKKPGASVKVSCKASGYIFS NYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 184
QVQLVQSGAEVKKPGASVKVSCKASGYIFS HYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 185
QVQLVQSGAEVKKPGASVKVSCKASGHIFS NYWIQ WVRQAPGQGLEWMG EILPGSGHTEYTENFKD
RVTMTRDTSTSTVYMELSSLRSEDTAVYYCAR YHFGSSPNWYFDV WGQGTLVTVSS
SEQ ID NO: 186
QVQLVQSGAEVKKPGASVKVSCKASGHIFS HYWIQ WVRQAPGQGLEWMG EILPGSGHTEYTENFKD
RVTMTRDTSTSTVYMELSSLRSEDTAVYYCAR YHFGSSPNWYFDV WGQGTLVTVSS
SEQ ID NO: 187
QVQLVQSGAEVKKPGASVKVSCKASGHIFS NYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 188
QVQLVQSGAEVKKPGASVKVSCKASGHIFS HYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 189
QVQLVQSGAEVKKPGASVKVSCKASGYTFS NYWIQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 190
QVQLVQSGAEVKKPGASVKVSCKASGYTFS HYWIQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 191
QVQLVQSGAEVKKPGASVKVSCKASGYTFS NYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 192
QVQLVQSGAEVKKPGASVKVSCKASGYTFS HYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 193
QVQLVQSGAEVKKPGASVKVSCKASGHTFS NYWIQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 194
QVQLVQSGAEVKKPGASVKVSCKASGHTFS HYWIQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 195
QVQLVQSGAEVKKPGASVKVSCKASGHTFS NYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 196
QVQLVQSGAEVKKPGASVKVSCKASGHTFS HYWHQ WVRQAPGQGLEWMG
EILPGSGHTEYTENFKD RVTMTRDTSTSTVYMELSSLRSEDTAVYYCAR YHFGSSPNWYFDV
WGQGTLVTVSS
SEQ ID NO: 197
QVQLVQSGAEVKKPGASVKVSCKASGYIFS NYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 198
QVQLVQSGAEVKKPGASVKVSCKASGYIFS HYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 199
QVQLVQSGAEVKKPGASVKVSCKASGYIFS NYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 200
QVQLVQSGAEVKKPGASVKVSCKASGYIFS HYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 201
QVQLVQSGAEVKKPGASVKVSCKASGHIFS NYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 202
QVQLVQSGAEVKKPGASVKVSCKASGHIFS HYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 203
QVQLVQSGAEVKKPGASVKVSCKASGHIFS NYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 204
QVQLVQSGAEVKKPGASVKVSCKASGHIFS HYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 205
QVQLVQSGAEVKKPGASVKVSCKASGYTFS NYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 206
QVQLVQSGAEVKKPGASVKVSCKASGYTFS HYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 207
QVQLVQSGAEVKKPGASVKVSCKASGYTFS NYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 208
QVQLVQSGAEVKKPGASVKVSCKASGYTFS HYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 209
QVQLVQSGAEVKKPGASVKVSCKASGHTFS NYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 210
QVQLVQSGAEVKKPGASVKVSCKASGHTFS HYWIQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 211
QVQLVQSGAEVKKPGASVKVSCKASGHTFS NYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 212
QVQLVQSGAEVKKPGASVKVSCKASGHTFS HYWHQ WVRQAPGQGLEWMG
EIHPGSGHTEYTENFKD RVTMTRDTSTSTVYMELSSLRSEDTAVYYCAR YHFGSSPNWYFDV
WGQGTLVTVSS
SEQ ID NO: 213
QVQLVQSGAEVKKPGASVKVSCKASGYIFS NYWIQ WVRQAPGQGLEWMG EILPGSGSTEYTENFKD
RVTMTRDTSISTAYMELSSLRSEDTAVYYCAR YHFGSSPNWYFDV WGQGTLVTVSS
SEQ ID NO: 214
QVQLVQSGAEVKKPGASVKVSCKASGYIFS HYWIQ WVRQAPGQGLEWMG EILPGSGSTEYTENFKD
RVTMTRDTSISTAYMELSSLRSEDTAVYYCAR YHFGSSPNWYFDV WGQGTLVTVSS
SEQ ID NO: 215
QVQLVQSGAEVKKPGASVKVSCKASGYIFS NYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 216
QVQLVQSGAEVKKPGASVKVSCKASGYIFS HYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 217
QVQLVQSGAEVKKPGASVKVSCKASGHIFS NYWIQ WVRQAPGQGLEWMG EILPGSGSTEYTENFKD
RVTMTRDTSISTAYMELSSLRSEDTAVYYCAR YHFGSSPNWYFDV WGQGTLVTVSS
SEQ ID NO: 218
QVQLVQSGAEVKKPGASVKVSCKASGHIFS HYWIQ WVRQAPGQGLEWMG EILPGSGSTEYTENFKD
RVTMTRDTSISTAYMELSSLRSEDTAVYYCAR YHFGSSPNWYFDV WGQGTLVTVSS
SEQ ID NO: 219
QVQLVQSGAEVKKPGASVKVSCKASGHIFS NYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 220
QVQLVQSGAEVKKPGASVKVSCKASGHIFS HYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 221
QVQLVQSGAEVKKPGASVKVSCKASGYTFS NYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 222
QVQLVQSGAEVKKPGASVKVSCKASGYTFS HYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 223
QVQLVQSGAEVKKPGASVKVSCKASGYTFS NYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 224
QVQLVQSGAEVKKPGASVKVSCKASGYTFS HYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 225
QVQLVQSGAEVKKPGASVKVSCKASGHTFS NYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 226
QVQLVQSGAEVKKPGASVKVSCKASGHTFS HYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 227
QVQLVQSGAEVKKPGASVKVSCKASGHTFS NYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 228
QVQLVQSGAEVKKPGASVKVSCKASGHTFS HYWHQ WVRQAPGQGLEWMG
EILPGSGSTEYTENFKD RVTMTRDTSISTAYMELSSLRSEDTAVYYCAR YHFGSSPNWYFDV
WGQGTLVTVSS
SEQ ID NO: 229
QVQLVQSGAEVKKPGASVKVSCKASGYIFS NYWIQ WVRQAPGQGLEWMG EIHPGSGSTEYTENFKD
RVTMTRDTSISTAYMELSSLRSEDTAVYYCAR YHFGSSPNWYFDV WGQGTLVTVSS
SEQ ID NO: 230
QVQLVQSGAEVKKPGASVKVSCKASGYIFS HYWIQ WVRQAPGQGLEWMG EIHPGSGSTEYTENFKD
RVTMTRDTSISTAYMELSSLRSEDTAVYYCAR YHFGSSPNWYFDV WGQGTLVTVSS
SEQ ID NO: 231
QVQLVQSGAEVKKPGASVKVSCKASGYIFS NYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 232
QVQLVQSGAEVKKPGASVKVSCKASGYIFS HYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 233
QVQLVQSGAEVKKPGASVKVSCKASGHIFS NYWIQ WVRQAPGQGLEWMG EIHPGSGSTEYTENFKD
RVTMTRDTSISTAYMELSSLRSEDTAVYYCAR YHFGSSPNWYFDV WGQGTLVTVSS
SEQ ID NO: 234
QVQLVQSGAEVKKPGASVKVSCKASGHIFS HYWIQ WVRQAPGQGLEWMG EIHPGSGSTEYTENFKD
RVTMTRDTSISTAYMELSSLRSEDTAVYYCAR YHFGSSPNWYFDV WGQGTLVTVSS
SEQ ID NO: 235
QVQLVQSGAEVKKPGASVKVSCKASGHIFS NYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 236
QVQLVQSGAEVKKPGASVKVSCKASGHIFS HYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 237
QVQLVQSGAEVKKPGASVKVSCKASGYTFS NYWIQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 238
QVQLVQSGAEVKKPGASVKVSCKASGYTFS HYWIQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 239
QVQLVQSGAEVKKPGASVKVSCKASGYTFS NYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 240
QVQLVQSGAEVKKPGASVKVSCKASGYTFS HYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 241
QVQLVQSGAEVKKPGASVKVSCKASGHTFS NYWIQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 242
QVQLVQSGAEVKKPGASVKVSCKASGHTFS HYWIQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 243
QVQLVQSGAEVKKPGASVKVSCKASGHTFS NYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 244
QVQLVQSGAEVKKPGASVKVSCKASGHTFS HYWHQ WVRQAPGQGLEWMG
EIHPGSGSTEYTENFKD RVTMTRDTSISTAYMELSSLRSEDTAVYYCAR YHFGSSPNWYFDV
WGQGTLVTVSS
SEQ ID NO: 245
QVQLVQSGAEVKKPGASVKVSCKASGYIFS NYWIQ WVRQAPGQGLEWMG EILPGSGHTEYTENFKD
RVTMTRDTSISTAYMELSSLRSEDTAVYYCAR YHFGSSPNWYFDV WGQGTLVTVSS
SEQ ID NO: 246
QVQLVQSGAEVKKPGASVKVSCKASGYIFS HYWIQ WVRQAPGQGLEWMG EILPGSGHTEYTENFKD
RVTMTRDTSISTAYMELSSLRSEDTAVYYCAR YHFGSSPNWYFDV WGQGTLVTVSS
SEQ ID NO: 247
QVQLVQSGAEVKKPGASVKVSCKASGYIFS NYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 248
QVQLVQSGAEVKKPGASVKVSCKASGYIFS HYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 249
QVQLVQSGAEVKKPGASVKVSCKASGHIFS NYWIQ WVRQAPGQGLEWMG EILPGSGHTEYTENFKD
RVTMTRDTSISTAYMELSSLRSEDTAVYYCAR YHFGSSPNWYFDV WGQGTLVTVSS
SEQ ID NO: 250
QVQLVQSGAEVKKPGASVKVSCKASGHIFS HYWIQ WVRQAPGQGLEWMG EILPGSGHTEYTENFKD
RVTMTRDTSISTAYMELSSLRSEDTAVYYCAR YHFGSSPNWYFDV WGQGTLVTVSS
SEQ ID NO: 251
QVQLVQSGAEVKKPGASVKVSCKASGHIFS NYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 252
QVQLVQSGAEVKKPGASVKVSCKASGHIFS HYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 253
QVQLVQSGAEVKKPGASVKVSCKASGYTFS NYWIQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 254
QVQLVQSGAEVKKPGASVKVSCKASGYTFS HYWIQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 255
QVQLVQSGAEVKKPGASVKVSCKASGYTFS NYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 256
QVQLVQSGAEVKKPGASVKVSCKASGYTFS HYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 257
QVQLVQSGAEVKKPGASVKVSCKASGHTFS NYWIQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 258
QVQLVQSGAEVKKPGASVKVSCKASGHTFS HYWIQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 259
QVQLVQSGAEVKKPGASVKVSCKASGHTFS NYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 260
QVQLVQSGAEVKKPGASVKVSCKASGHTFS HYWHQ WVRQAPGQGLEWMG
EILPGSGHTEYTENFKD RVTMTRDTSISTAYMELSSLRSEDTAVYYCAR YHFGSSPNWYFDV
WGQGTLVTVSS
SEQ ID NO: 261
QVQLVQSGAEVKKPGASVKVSCKASGYIFS NYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 262
QVQLVQSGAEVKKPGASVKVSCKASGYIFS HYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 263
QVQLVQSGAEVKKPGASVKVSCKASGYIFS NYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 264
QVQLVQSGAEVKKPGASVKVSCKASGYIFS HYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 265
QVQLVQSGAEVKKPGASVKVSCKASGHIFS NYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 266
QVQLVQSGAEVKKPGASVKVSCKASGHIFS HYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 267
QVQLVQSGAEVKKPGASVKVSCKASGHIFS NYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 268
QVQLVQSGAEVKKPGASVKVSCKASGHIFS HYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 269
QVQLVQSGAEVKKPGASVKVSCKASGYTFS NYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 270
QVQLVQSGAEVKKPGASVKVSCKASGYTFS HYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 271
QVQLVQSGAEVKKPGASVKVSCKASGYTFS NYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 272
QVQLVQSGAEVKKPGASVKVSCKASGYTFS HYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 273
QVQLVQSGAEVKKPGASVKVSCKASGHTFS NYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 274
QVQLVQSGAEVKKPGASVKVSCKASGHTFS HYWIQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 275
QVQLVQSGAEVKKPGASVKVSCKASGHTFS NYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 276
QVQLVQSGAEVKKPGASVKVSCKASGHTFS HYWHQ WVRQAPGQGLEWMG
EIHPGSGHTEYTENFKD RVTMTRDTSISTAYMELSSLRSEDTAVYYCAR YHFGSSPNWYFDV
WGQGTLVTVSS
SEQ ID NO: 277
DIQMTQSPSSLSASVGDRVTITC
SEQ ID NO: 278
GASENIYGALN
SEQ ID NO: 279
GAHENIYGALN
SEQ ID NO: 280
GASENIYHALN
SEQ ID NO: 281
GASENIYGAHN
SEQ ID NO: 282
GAHENIYHALN
SEQ ID NO: 283
GAHENIYGAHN
SEQ ID NO: 284
GASENIYHAHN
SEQ ID NO: 285
GAHENIYHAHN
SEQ ID NO: 286
WYQQKPGKAPKLLIY
SEQ ID NO: 287
GATNLAD
SEQ ID NO: 288
GATNLAH
SEQ ID NO: 289
GVPSRFSGSGSGTDFTLTISSLQPEDFATYYC
SEQ ID NO: 290
QNVLNTPLT
SEQ ID NO: 291
QNHLNTPLT
SEQ ID NO: 292
QNVLHTPLT
SEQ ID NO: 293
QNVLNHPLT
SEQ ID NO: 294
QNHLHTPLT
SEQ ID NO: 295
QNHLNHPLT
SEQ ID NO: 296
QNVLHHPLT
SEQ ID NO: 297
QNHLHHPLT
SEQ ID NO: 298
FGQGTKVEIKR
SEQ ID NO: 299
DIQMTQSPSSLSASVGDRVTITC GASENIYGALN WYQQKPGKAPKLLIY GATNLAD
GVPSRFSGSGSGTDFTLTISSLQPEDFATYYC QNVLNTPLT FGQGTKVEIKR
SEQ ID NO: 300
DIQMTQSPSSLSASVGDRVTITC GAHENIYGALN WYQQKPGKAPKLLIY GATNLAD
GVPSRFSGSGSGTDFTLTISSLQPEDFATYYC QNVLNTPLT FGQGTKVEIKR
SEQ ID NO: 301
DIQMTQSPSSLSASVGDRVTITC GASENIYHALN WYQQKPGKAPKLLIY GATNLAD
GVPSRFSGSGSGTDFTLTISSLQPEDFATYYC QNVLNTPLT FGQGTKVEIKR
SEQ ID NO: 302
DIQMTQSPSSLSASVGDRVTITC GASENIYGAHN WYQQKPGKAPKLLIY GATNLAD
GVPSRFSGSGSGTDFTLTISSLQPEDFATYYC QNVLNTPLT FGQGTKVEIKR
SEQ ID NO: 303
DIQMTQSPSSLSASVGDRVTITC GAHENIYHALN WYQQKPGKAPKLLIY GATNLAD
GVPSRFSGSGSGTDFTLTISSLQPEDFATYYC QNVLNTPLT FGQGTKVEIKR
SEQ ID NO: 304
DIQMTQSPSSLSASVGDRVTITC GAHENIYGAHN WYQQKPGKAPKLLIY GATNLAD
GVPSRFSGSGSGTDFTLTISSLQPEDFATYYC QNVLNTPLT FGQGTKVEIKR
SEQ ID NO: 305
DIQMTQSPSSLSASVGDRVTITC GASENIYHAHN WYQQKPGKAPKLLIY GATNLAD
GVPSRFSGSGSGTDFTLTISSLQPEDFATYYC QNVLNTPLT FGQGTKVEIKR
SEQ ID NO: 306
DIQMTQSPSSLSASVGDRVTITC GAHENIYHAHN WYQQKPGKAPKLLIY GATNLAD
GVPSRFSGSGSGTDFTLTISSLQPEDFATYYC QNVLNTPLT FGQGTKVEIKR
SEQ ID NO: 307
DIQMTQSPSSLSASVGDRVTITC GASENIYGALN WYQQKPGKAPKLLIY GATNLAH
GVPSRFSGSGSGTDFTLTISSLQPEDFATYYC QNVLNTPLT FGQGTKVEIKR
SEQ ID NO: 308
DIQMTQSPSSLSASVGDRVTITC GAHENIYGALN WYQQKPGKAPKLLIY GATNLAH
GVPSRFSGSGSGTDFTLTISSLQPEDFATYYC QNVLNTPLT FGQGTKVEIKR
SEQ ID NO: 309
DIQMTQSPSSLSASVGDRVTITC GASENIYHALN WYQQKPGKAPKLLIY GATNLAH
GVPSRFSGSGSGTDFTLTISSLQPEDFATYYC QNVLNTPLT FGQGTKVEIKR
SEQ ID NO: 310
DIQMTQSPSSLSASVGDRVTITC GASENIYGAHN WYQQKPGKAPKLLIY GATNLAH
GVPSRFSGSGSGTDFTLTISSLQPEDFATYYC QNVLNTPLT FGQGTKVEIKR
SEQ ID NO: 311
DIQMTQSPSSLSASVGDRVTITC GAHENIYHALN WYQQKPGKAPKLLIY GATNLAH
GVPSRFSGSGSGTDFTLTISSLQPEDFATYYC QNVLNTPLT FGQGTKVEIKR
SEQ ID NO: 312
DIQMTQSPSSLSASVGDRVTITC GAHENIYGAHN WYQQKPGKAPKLLIY GATNLAH
GVPSRFSGSGSGTDFTLTISSLQPEDFATYYC QNVLNTPLT FGQGTKVEIKR
SEQ ID NO: 313
DIQMTQSPSSLSASVGDRVTITC GASENIYHAHN WYQQKPGKAPKLLIY GATNLAH
GVPSRFSGSGSGTDFTLTISSLQPEDFATYYC QNVLNTPLT FGQGTKVEIKR
SEQ ID NO: 314
DIQMTQSPSSLSASVGDRVTITC GAHENIYHAHN WYQQKPGKAPKLLIY GATNLAH
GVPSRFSGSGSGTDFTLTISSLQPEDFATYYC QNVLNTPLT FGQGTKVEIKR
SEQ ID NO: 315
DIQMTQSPSSLSASVGDRVTITC GASENIYGALN WYQQKPGKAPKLLIY GATNLAD
GVPSRFSGSGSGTDFTLTISSLQPEDFATYYC QNHLNTPLT FGQGTKVEIKR
SEQ ID NO: 316
DIQMTQSPSSLSASVGDRVTITC GAHENIYGALN WYQQKPGKAPKLLIY GATNLAD
GVPSRFSGSGSGTDFTLTISSLQPEDFATYYC QNHLNTPLT FGQGTKVEIKR
SEQ ID NO: 317
DIQMTQSPSSLSASVGDRVTITC GASENIYHALN WYQQKPGKAPKLLIY GATNLAD
GVPSRFSGSGSGTDFTLTISSLQPEDFATYYC QNHLNTPLT FGQGTKVEIKR
SEQ ID NO: 318
DIQMTQSPSSLSASVGDRVTITC GASENIYGAHN WYQQKPGKAPKLLIY GATNLAD
GVPSRFSGSGSGTDFTLTISSLQPEDFATYYC QNHLNTPLT FGQGTKVEIKR
SEQ ID NO: 319
DIQMTQSPSSLSASVGDRVTITC GAHENIYHALN WYQQKPGKAPKLLIY GATNLAD
GVPSRFSGSGSGTDFTLTISSLQPEDFATYYC QNHLNTPLT FGQGTKVEIKR
SEQ ID NO: 320
DIQMTQSPSSLSASVGDRVTITC GAHENIYGAHN WYQQKPGKAPKLLIY GATNLAD
GVPSRFSGSGSGTDFTLTISSLQPEDFATYYC QNHLNTPLT FGQGTKVEIKR
SEQ ID NO: 321
DIQMTQSPSSLSASVGDRVTITC GASENIYHAHN WYQQKPGKAPKLLIY GATNLAD
GVPSRFSGSGSGTDFTLTISSLQPEDFATYYC QNHLNTPLT FGQGTKVEIKR
SEQ ID NO: 322
DIQMTQSPSSLSASVGDRVTITC GAHENIYHAHN WYQQKPGKAPKLLIY GATNLAD
GVPSRFSGSGSGTDFTLTISSLQPEDFATYYC QNHLNTPLT FGQGTKVEIKR
SEQ ID NO: 323
DIQMTQSPSSLSASVGDRVTITC GASENIYGALN WYQQKPGKAPKLLIY GATNLAH
GVPSRFSGSGSGTDFTLTISSLQPEDFATYYC QNHLNTPLT FGQGTKVEIKR
SEQ ID NO: 324
DIQMTQSPSSLSASVGDRVTITC GAHENIYGALN WYQQKPGKAPKLLIY GATNLAH
GVPSRFSGSGSGTDFTLTISSLQPEDFATYYC QNHLNTPLT FGQGTKVEIKR
SEQ ID NO: 325
DIQMTQSPSSLSASVGDRVTITC GASENIYHALN WYQQKPGKAPKLLIY GATNLAH
GVPSRFSGSGSGTDFTLTISSLQPEDFATYYC QNHLNTPLT FGQGTKVEIKR
SEQ ID NO: 326
DIQMTQSPSSLSASVGDRVTITC GASENIYGAHN WYQQKPGKAPKLLIY GATNLAH
GVPSRFSGSGSGTDFTLTISSLQPEDFATYYC QNHLNTPLT FGQGTKVEIKR
SEQ ID NO: 327
DIQMTQSPSSLSASVGDRVTITC GAHENIYHALN WYQQKPGKAPKLLIY GATNLAH
GVPSRFSGSGSGTDFTLTISSLQPEDFATYYC QNHLNTPLT FGQGTKVEIKR
SEQ ID NO: 328
DIQMTQSPSSLSASVGDRVTITC GAHENIYGAHN WYQQKPGKAPKLLIY GATNLAH
GVPSRFSGSGSGTDFTLTISSLQPEDFATYYC QNHLNTPLT FGQGTKVEIKR
SEQ ID NO: 329
DIQMTQSPSSLSASVGDRVTITC GASENIYHAHN WYQQKPGKAPKLLIY GATNLAH
GVPSRFSGSGSGTDFTLTISSLQPEDFATYYC QNHLNTPLT FGQGTKVEIKR
SEQ ID NO: 330
DIQMTQSPSSLSASVGDRVTITC GAHENIYHAHN WYQQKPGKAPKLLIY GATNLAH
GVPSRFSGSGSGTDFTLTISSLQPEDFATYYC QNHLNTPLT FGQGTKVEIKR
SEQ ID NO: 331
DIQMTQSPSSLSASVGDRVTITC GASENIYGALN WYQQKPGKAPKLLIY GATNLAD
GVPSRFSGSGSGTDFTLTISSLQPEDFATYYC QNVLHTPLT FGQGTKVEIKR
SEQ ID NO: 332
DIQMTQSPSSLSASVGDRVTITC GAHENIYGALN WYQQKPGKAPKLLIY GATNLAD
GVPSRFSGSGSGTDFTLTISSLQPEDFATYYC QNVLHTPLT FGQGTKVEIKR
SEQ ID NO: 333
DIQMTQSPSSLSASVGDRVTITC GASENIYHALN WYQQKPGKAPKLLIY GATNLAD
GVPSRFSGSGSGTDFTLTISSLQPEDFATYYC QNVLHTPLT FGQGTKVEIKR
SEQ ID NO: 334
DIQMTQSPSSLSASVGDRVTITC GASENIYGAHN WYQQKPGKAPKLLIY GATNLAD
GVPSRFSGSGSGTDFTLTISSLQPEDFATYYC QNVLHTPLT FGQGTKVEIKR
SEQ ID NO: 335
DIQMTQSPSSLSASVGDRVTITC GAHENIYHALN WYQQKPGKAPKLLIY GATNLAD
GVPSRFSGSGSGTDFTLTISSLQPEDFATYYC QNVLHTPLT FGQGTKVEIKR
SEQ ID NO: 336
DIQMTQSPSSLSASVGDRVTITC GAHENIYGAHN WYQQKPGKAPKLLIY GATNLAD
GVPSRFSGSGSGTDFTLTISSLQPEDFATYYC QNVLHTPLT FGQGTKVEIKR
SEQ ID NO: 337
DIQMTQSPSSLSASVGDRVTITC GASENIYHAHN WYQQKPGKAPKLLIY GATNLAD
GVPSRFSGSGSGTDFTLTISSLQPEDFATYYC QNVLHTPLT FGQGTKVEIKR
SEQ ID NO: 338
DIQMTQSPSSLSASVGDRVTITC GAHENIYHAHN WYQQKPGKAPKLLIY GATNLAD
GVPSRFSGSGSGTDFTLTISSLQPEDFATYYC QNVLHTPLT FGQGTKVEIKR
SEQ ID NO: 339
DIQMTQSPSSLSASVGDRVTITC GASENIYGALN WYQQKPGKAPKLLIY GATNLAH
GVPSRFSGSGSGTDFTLTISSLQPEDFATYYC QNVLHTPLT FGQGTKVEIKR
SEQ ID NO: 340
DIQMTQSPSSLSASVGDRVTITC GAHENIYGALN WYQQKPGKAPKLLIY GATNLAH
GVPSRFSGSGSGTDFTLTISSLQPEDFATYYC QNVLHTPLT FGQGTKVEIKR
SEQ ID NO: 341
DIQMTQSPSSLSASVGDRVTITC GASENIYHALN WYQQKPGKAPKLLIY GATNLAH
GVPSRFSGSGSGTDFTLTISSLQPEDFATYYC QNVLHTPLT FGQGTKVEIKR
SEQ ID NO: 342
DIQMTQSPSSLSASVGDRVTITC GASENIYGAHN WYQQKPGKAPKLLIY GATNLAH
GVPSRFSGSGSGTDFTLTISSLQPEDFATYYC QNVLHTPLT FGQGTKVEIKR
SEQ ID NO: 343
DIQMTQSPSSLSASVGDRVTITC GAHENIYHALN WYQQKPGKAPKLLIY GATNLAH
GVPSRFSGSGSGTDFTLTISSLQPEDFATYYC QNVLHTPLT FGQGTKVEIKR
SEQ ID NO: 344
DIQMTQSPSSLSASVGDRVTITC GAHENIYGAHN WYQQKPGKAPKLLIY GATNLAH
GVPSRFSGSGSGTDFTLTISSLQPEDFATYYC QNVLHTPLT FGQGTKVEIKR
SEQ ID NO: 345
DIQMTQSPSSLSASVGDRVTITC GASENIYHAHN WYQQKPGKAPKLLIY GATNLAH
GVPSRFSGSGSGTDFTLTISSLQPEDFATYYC QNVLHTPLT FGQGTKVEIKR
SEQ ID NO: 346
DIQMTQSPSSLSASVGDRVTITC GAHENIYHAHN WYQQKPGKAPKLLIY GATNLAH
GVPSRFSGSGSGTDFTLTISSLQPEDFATYYC QNVLHTPLT FGQGTKVEIKR
SEQ ID NO: 347
DIQMTQSPSSLSASVGDRVTITC GASENIYGALN WYQQKPGKAPKLLIY GATNLAD
GVPSRFSGSGSGTDFTLTISSLQPEDFATYYC QNVLNHPLT FGQGTKVEIKR
SEQ ID NO: 348
DIQMTQSPSSLSASVGDRVTITC GAHENIYGALN WYQQKPGKAPKLLIY GATNLAD
GVPSRFSGSGSGTDFTLTISSLQPEDFATYYC QNVLNHPLT FGQGTKVEIKR
SEQ ID NO: 349
DIQMTQSPSSLSASVGDRVTITC GASENIYHALN WYQQKPGKAPKLLIY GATNLAD
GVPSRFSGSGSGTDFTLTISSLQPEDFATYYC QNVLNHPLT FGQGTKVEIKR
SEQ ID NO: 350
DIQMTQSPSSLSASVGDRVTITC GASENIYGAHN WYQQKPGKAPKLLIY GATNLAD
GVPSRFSGSGSGTDFTLTISSLQPEDFATYYC QNVLNHPLT FGQGTKVEIKR
SEQ ID NO: 351
DIQMTQSPSSLSASVGDRVTITC GAHENIYHALN WYQQKPGKAPKLLIY GATNLAD
GVPSRFSGSGSGTDFTLTISSLQPEDFATYYC QNVLNHPLT FGQGTKVEIKR
SEQ ID NO: 352
DIQMTQSPSSLSASVGDRVTITC GAHENIYGAHN WYQQKPGKAPKLLIY GATNLAD
GVPSRFSGSGSGTDFTLTISSLQPEDFATYYC QNVLNHPLT FGQGTKVEIKR
SEQ ID NO: 353
DIQMTQSPSSLSASVGDRVTITC GASENIYHAHN WYQQKPGKAPKLLIY GATNLAD
GVPSRFSGSGSGTDFTLTISSLQPEDFATYYC QNVLNHPLT FGQGTKVEIKR
SEQ ID NO: 354
DIQMTQSPSSLSASVGDRVTITC GAHENIYHAHN WYQQKPGKAPKLLIY GATNLAD
GVPSRFSGSGSGTDFTLTISSLQPEDFATYYC QNVLNHPLT FGQGTKVEIKR
SEQ ID NO: 355
DIQMTQSPSSLSASVGDRVTITC GASENIYGALN WYQQKPGKAPKLLIY GATNLAH
GVPSRFSGSGSGTDFTLTISSLQPEDFATYYC QNVLNHPLT FGQGTKVEIKR
SEQ ID NO: 356
DIQMTQSPSSLSASVGDRVTITC GAHENIYGALN WYQQKPGKAPKLLIY GATNLAH
GVPSRFSGSGSGTDFTLTISSLQPEDFATYYC QNVLNHPLT FGQGTKVEIKR
SEQ ID NO: 357
DIQMTQSPSSLSASVGDRVTITC GASENIYHALN WYQQKPGKAPKLLIY GATNLAH
GVPSRFSGSGSGTDFTLTISSLQPEDFATYYC QNVLNHPLT FGQGTKVEIKR
SEQ ID NO: 358
DIQMTQSPSSLSASVGDRVTITC GASENIYGAHN WYQQKPGKAPKLLIY GATNLAH
GVPSRFSGSGSGTDFTLTISSLQPEDFATYYC QNVLNHPLT FGQGTKVEIKR
SEQ ID NO: 359
DIQMTQSPSSLSASVGDRVTITC GAHENIYHALN WYQQKPGKAPKLLIY GATNLAH
GVPSRFSGSGSGTDFTLTISSLQPEDFATYYC QNVLNHPLT FGQGTKVEIKR
SEQ ID NO: 360
DIQMTQSPSSLSASVGDRVTITC GAHENIYGAHN WYQQKPGKAPKLLIY GATNLAH
GVPSRFSGSGSGTDFTLTISSLQPEDFATYYC QNVLNHPLT FGQGTKVEIKR
SEQ ID NO: 361
DIQMTQSPSSLSASVGDRVTITC GASENIYHAHN WYQQKPGKAPKLLIY GATNLAH
GVPSRFSGSGSGTDFTLTISSLQPEDFATYYC QNVLNHPLT FGQGTKVEIKR
SEQ ID NO: 362
DIQMTQSPSSLSASVGDRVTITC GAHENIYHAHN WYQQKPGKAPKLLIY GATNLAH
GVPSRFSGSGSGTDFTLTISSLQPEDFATYYC QNVLNHPLT FGQGTKVEIKR
SEQ ID NO: 363
DIQMTQSPSSLSASVGDRVTITC GASENIYGALN WYQQKPGKAPKLLIY GATNLAD
GVPSRFSGSGSGTDFTLTISSLQPEDFATYYC QNHLHTPLT FGQGTKVEIKR
SEQ ID NO: 364
DIQMTQSPSSLSASVGDRVTITC GAHENIYGALN WYQQKPGKAPKLLIY GATNLAD
GVPSRFSGSGSGTDFTLTISSLQPEDFATYYC QNHLHTPLT FGQGTKVEIKR
SEQ ID NO: 365
DIQMTQSPSSLSASVGDRVTITC GASENIYHALN WYQQKPGKAPKLLIY GATNLAD
GVPSRFSGSGSGTDFTLTISSLQPEDFATYYC QNHLHTPLT FGQGTKVEIKR
SEQ ID NO: 366
DIQMTQSPSSLSASVGDRVTITC GASENIYGAHN WYQQKPGKAPKLLIY GATNLAD
GVPSRFSGSGSGTDFTLTISSLQPEDFATYYC QNHLHTPLT FGQGTKVEIKR
SEQ ID NO: 367
DIQMTQSPSSLSASVGDRVTITC GAHENIYHALN WYQQKPGKAPKLLIY GATNLAD
GVPSRFSGSGSGTDFTLTISSLQPEDFATYYC QNHLHTPLT FGQGTKVEIKR
SEQ ID NO: 368
DIQMTQSPSSLSASVGDRVTITC GAHENIYGAHN WYQQKPGKAPKLLIY GATNLAD
GVPSRFSGSGSGTDFTLTISSLQPEDFATYYC QNHLHTPLT FGQGTKVEIKR
SEQ ID NO: 369
DIQMTQSPSSLSASVGDRVTITC GASENIYHAHN WYQQKPGKAPKLLIY GATNLAD
GVPSRFSGSGSGTDFTLTISSLQPEDFATYYC QNHLHTPLT FGQGTKVEIKR
SEQ ID NO: 370
DIQMTQSPSSLSASVGDRVTITC GAHENIYHAHN WYQQKPGKAPKLLIY GATNLAD
GVPSRFSGSGSGTDFTLTISSLQPEDFATYYC QNHLHTPLT FGQGTKVEIKR
SEQ ID NO: 371
DIQMTQSPSSLSASVGDRVTITC GASENIYGALN WYQQKPGKAPKLLIY GATNLAH
GVPSRFSGSGSGTDFTLTISSLQPEDFATYYC QNHLHTPLT FGQGTKVEIKR
SEQ ID NO: 372
DIQMTQSPSSLSASVGDRVTITC GAHENIYGALN WYQQKPGKAPKLLIY GATNLAH
GVPSRFSGSGSGTDFTLTISSLQPEDFATYYC QNHLHTPLT FGQGTKVEIKR
SEQ ID NO: 373
DIQMTQSPSSLSASVGDRVTITC GASENIYHALN WYQQKPGKAPKLLIY GATNLAH
GVPSRFSGSGSGTDFTLTISSLQPEDFATYYC QNHLHTPLT FGQGTKVEIKR
SEQ ID NO: 374
DIQMTQSPSSLSASVGDRVTITC GASENIYGAHN WYQQKPGKAPKLLIY GATNLAH
GVPSRFSGSGSGTDFTLTISSLQPEDFATYYC QNHLHTPLT FGQGTKVEIKR
SEQ ID NO: 375
DIQMTQSPSSLSASVGDRVTITC GAHENIYHALN WYQQKPGKAPKLLIY GATNLAH
GVPSRFSGSGSGTDFTLTISSLQPEDFATYYC QNHLHTPLT FGQGTKVEIKR
SEQ ID NO: 376
DIQMTQSPSSLSASVGDRVTITC GAHENIYGAHN WYQQKPGKAPKLLIY GATNLAH
GVPSRFSGSGSGTDFTLTISSLQPEDFATYYC QNHLHTPLT FGQGTKVEIKR
SEQ ID NO: 377
DIQMTQSPSSLSASVGDRVTITC GASENIYHAHN WYQQKPGKAPKLLIY GATNLAH
GVPSRFSGSGSGTDFTLTISSLQPEDFATYYC QNHLHTPLT FGQGTKVEIKR
SEQ ID NO: 378
DIQMTQSPSSLSASVGDRVTITC GAHENIYHAHN WYQQKPGKAPKLLIY GATNLAH
GVPSRFSGSGSGTDFTLTISSLQPEDFATYYC QNHLHTPLT FGQGTKVEIKR
SEQ ID NO: 379
DIQMTQSPSSLSASVGDRVTITC GASENIYGALN WYQQKPGKAPKLLIY GATNLAD
GVPSRFSGSGSGTDFTLTISSLQPEDFATYYC QNHLNHPLT FGQGTKVEIKR
SEQ ID NO: 380
DIQMTQSPSSLSASVGDRVTITC GAHENIYGALN WYQQKPGKAPKLLIY GATNLAD
GVPSRFSGSGSGTDFTLTISSLQPEDFATYYC QNHLNHPLT FGQGTKVEIKR
SEQ ID NO: 381
DIQMTQSPSSLSASVGDRVTITC GASENIYHALN WYQQKPGKAPKLLIY GATNLAD
GVPSRFSGSGSGTDFTLTISSLQPEDFATYYC QNHLNHPLT FGQGTKVEIKR
SEQ ID NO: 382
DIQMTQSPSSLSASVGDRVTITC GASENIYGAHN WYQQKPGKAPKLLIY GATNLAD
GVPSRFSGSGSGTDFTLTISSLQPEDFATYYC QNHLNHPLT FGQGTKVEIKR
SEQ ID NO: 383
DIQMTQSPSSLSASVGDRVTITC GAHENIYHALN WYQQKPGKAPKLLIY GATNLAD
GVPSRFSGSGSGTDFTLTISSLQPEDFATYYC QNHLNHPLT FGQGTKVEIKR
SEQ ID NO: 384
DIQMTQSPSSLSASVGDRVTITC GAHENIYGAHN WYQQKPGKAPKLLIY GATNLAD
GVPSRFSGSGSGTDFTLTISSLQPEDFATYYC QNHLNHPLT FGQGTKVEIKR
SEQ ID NO: 385
DIQMTQSPSSLSASVGDRVTITC GASENIYHAHN WYQQKPGKAPKLLIY GATNLAD
GVPSRFSGSGSGTDFTLTISSLQPEDFATYYC QNHLNHPLT FGQGTKVEIKR
SEQ ID NO: 386
DIQMTQSPSSLSASVGDRVTITC GAHENIYHAHN WYQQKPGKAPKLLIY GATNLAD
GVPSRFSGSGSGTDFTLTISSLQPEDFATYYC QNHLNHPLT FGQGTKVEIKR
SEQ ID NO: 387
DIQMTQSPSSLSASVGDRVTITC GASENIYGALN WYQQKPGKAPKLLIY GATNLAH
GVPSRFSGSGSGTDFTLTISSLQPEDFATYYC QNHLNHPLT FGQGTKVEIKR
SEQ ID NO: 388
DIQMTQSPSSLSASVGDRVTITC GAHENIYGALN WYQQKPGKAPKLLIY GATNLAH
GVPSRFSGSGSGTDFTLTISSLQPEDFATYYC QNHLNHPLT FGQGTKVEIKR
SEQ ID NO: 389
DIQMTQSPSSLSASVGDRVTITC GASENIYHALN WYQQKPGKAPKLLIY GATNLAH
GVPSRFSGSGSGTDFTLTISSLQPEDFATYYC QNHLNHPLT FGQGTKVEIKR
SEQ ID NO: 390
DIQMTQSPSSLSASVGDRVTITC GASENIYGAHN WYQQKPGKAPKLLIY GATNLAH
GVPSRFSGSGSGTDFTLTISSLQPEDFATYYC QNHLNHPLT FGQGTKVEIKR
SEQ ID NO: 391
DIQMTQSPSSLSASVGDRVTITC GAHENIYHALN WYQQKPGKAPKLLIY GATNLAH
GVPSRFSGSGSGTDFTLTISSLQPEDFATYYC QNHLNHPLT FGQGTKVEIKR
SEQ ID NO: 392
DIQMTQSPSSLSASVGDRVTITC GAHENIYGAHN WYQQKPGKAPKLLIY GATNLAH
GVPSRFSGSGSGTDFTLTISSLQPEDFATYYC QNHLNHPLT FGQGTKVEIKR
SEQ ID NO: 393
DIQMTQSPSSLSASVGDRVTITC GASENIYHAHN WYQQKPGKAPKLLIY GATNLAH
GVPSRFSGSGSGTDFTLTISSLQPEDFATYYC QNHLNHPLT FGQGTKVEIKR
SEQ ID NO: 394
DIQMTQSPSSLSASVGDRVTITC GAHENIYHAHN WYQQKPGKAPKLLIY GATNLAH
GVPSRFSGSGSGTDFTLTISSLQPEDFATYYC QNHLNHPLT FGQGTKVEIKR
SEQ ID NO: 395
DIQMTQSPSSLSASVGDRVTITC GASENIYGALN WYQQKPGKAPKLLIY GATNLAD
GVPSRFSGSGSGTDFTLTISSLQPEDFATYYC QNVLHHPLT FGQGTKVEIKR
SEQ ID NO: 396
DIQMTQSPSSLSASVGDRVTITC GAHENIYGALN WYQQKPGKAPKLLIY GATNLAD
GVPSRFSGSGSGTDFTLTISSLQPEDFATYYC QNVLHHPLT FGQGTKVEIKR
SEQ ID NO: 397
DIQMTQSPSSLSASVGDRVTITC GASENIYHALN WYQQKPGKAPKLLIY GATNLAD
GVPSRFSGSGSGTDFTLTISSLQPEDFATYYC QNVLHHPLT FGQGTKVEIKR
SEQ ID NO: 398
DIQMTQSPSSLSASVGDRVTITC GASENIYGAHN WYQQKPGKAPKLLIY GATNLAD
GVPSRFSGSGSGTDFTLTISSLQPEDFATYYC QNVLHHPLT FGQGTKVEIKR
SEQ ID NO: 399
DIQMTQSPSSLSASVGDRVTITC GAHENIYHALN WYQQKPGKAPKLLIY GATNLAD
GVPSRFSGSGSGTDFTLTISSLQPEDFATYYC QNVLHHPLT FGQGTKVEIKR
SEQ ID NO: 400
DIQMTQSPSSLSASVGDRVTITC GAHENIYGAHN WYQQKPGKAPKLLIY GATNLAD
GVPSRFSGSGSGTDFTLTISSLQPEDFATYYC QNVLHHPLT FGQGTKVEIKR
SEQ ID NO: 401
DIQMTQSPSSLSASVGDRVTITC GASENIYHAHN WYQQKPGKAPKLLIY GATNLAD
GVPSRFSGSGSGTDFTLTISSLQPEDFATYYC QNVLHHPLT FGQGTKVEIKR
SEQ ID NO: 402
DIQMTQSPSSLSASVGDRVTITC GAHENIYHAHN WYQQKPGKAPKLLIY GATNLAD
GVPSRFSGSGSGTDFTLTISSLQPEDFATYYC QNVLHHPLT FGQGTKVEIKR
SEQ ID NO: 403
DIQMTQSPSSLSASVGDRVTITC GASENIYGALN WYQQKPGKAPKLLIY GATNLAH
GVPSRFSGSGSGTDFTLTISSLQPEDFATYYC QNVLHHPLT FGQGTKVEIKR
SEQ ID NO: 404
DIQMTQSPSSLSASVGDRVTITC GAHENIYGALN WYQQKPGKAPKLLIY GATNLAH
GVPSRFSGSGSGTDFTLTISSLQPEDFATYYC QNVLHHPLT FGQGTKVEIKR
SEQ ID NO: 405
DIQMTQSPSSLSASVGDRVTITC GASENIYHALN WYQQKPGKAPKLLIY GATNLAH
GVPSRFSGSGSGTDFTLTISSLQPEDFATYYC QNVLHHPLT FGQGTKVEIKR
SEQ ID NO: 406
DIQMTQSPSSLSASVGDRVTITC GASENIYGAHN WYQQKPGKAPKLLIY GATNLAH
GVPSRFSGSGSGTDFTLTISSLQPEDFATYYC QNVLHHPLT FGQGTKVEIKR
SEQ ID NO: 407
DIQMTQSPSSLSASVGDRVTITC GAHENIYHALN WYQQKPGKAPKLLIY GATNLAH
GVPSRFSGSGSGTDFTLTISSLQPEDFATYYC QNVLHHPLT FGQGTKVEIKR
SEQ ID NO: 408
DIQMTQSPSSLSASVGDRVTITC GAHENIYGAHN WYQQKPGKAPKLLIY GATNLAH
GVPSRFSGSGSGTDFTLTISSLQPEDFATYYC QNVLHHPLT FGQGTKVEIKR
SEQ ID NO: 409
DIQMTQSPSSLSASVGDRVTITC GASENIYHAHN WYQQKPGKAPKLLIY GATNLAH
GVPSRFSGSGSGTDFTLTISSLQPEDFATYYC QNVLHHPLT FGQGTKVEIKR
SEQ ID NO: 410
DIQMTQSPSSLSASVGDRVTITC GAHENIYHAHN WYQQKPGKAPKLLIY GATNLAH
GVPSRFSGSGSGTDFTLTISSLQPEDFATYYC QNVLHHPLT FGQGTKVEIKR
SEQ ID NO: 411
DIQMTQSPSSLSASVGDRVTITC GASENIYGALN WYQQKPGKAPKLLIY GATNLAD
GVPSRFSGSGSGTDFTLTISSLQPEDFATYYC QNHLHHPLT FGQGTKVEIKR
SEQ ID NO: 412
DIQMTQSPSSLSASVGDRVTITC GAHENIYGALN WYQQKPGKAPKLLIY GATNLAD
GVPSRFSGSGSGTDFTLTISSLQPEDFATYYC QNHLHHPLT FGQGTKVEIKR
SEQ ID NO: 413
DIQMTQSPSSLSASVGDRVTITC GASENIYHALN WYQQKPGKAPKLLIY GATNLAD
GVPSRFSGSGSGTDFTLTISSLQPEDFATYYC QNHLHHPLT FGQGTKVEIKR
SEQ ID NO: 414
DIQMTQSPSSLSASVGDRVTITC GASENIYGAHN WYQQKPGKAPKLLIY GATNLAD
GVPSRFSGSGSGTDFTLTISSLQPEDFATYYC QNHLHHPLT FGQGTKVEIKR
SEQ ID NO: 415
DIQMTQSPSSLSASVGDRVTITC GAHENIYHALN WYQQKPGKAPKLLIY GATNLAD
GVPSRFSGSGSGTDFTLTISSLQPEDFATYYC QNHLHHPLT FGQGTKVEIKR
SEQ ID NO: 416
DIQMTQSPSSLSASVGDRVTITC GAHENIYGAHN WYQQKPGKAPKLLIY GATNLAD
GVPSRFSGSGSGTDFTLTISSLQPEDFATYYC QNHLHHPLT FGQGTKVEIKR
SEQ ID NO: 417
DIQMTQSPSSLSASVGDRVTITC GASENIYHAHN WYQQKPGKAPKLLIY GATNLAD
GVPSRFSGSGSGTDFTLTISSLQPEDFATYYC QNHLHHPLT FGQGTKVEIKR
SEQ ID NO: 418
DIQMTQSPSSLSASVGDRVTITC GAHENIYHAHN WYQQKPGKAPKLLIY GATNLAD
GVPSRFSGSGSGTDFTLTISSLQPEDFATYYC QNHLHHPLT FGQGTKVEIKR
SEQ ID NO: 419
DIQMTQSPSSLSASVGDRVTITC GASENIYGALN WYQQKPGKAPKLLIY GATNLAH
GVPSRFSGSGSGTDFTLTISSLQPEDFATYYC QNHLHHPLT FGQGTKVEIKR
SEQ ID NO: 420
DIQMTQSPSSLSASVGDRVTITC GAHENIYGALN WYQQKPGKAPKLLIY GATNLAH
GVPSRFSGSGSGTDFTLTISSLQPEDFATYYC QNHLHHPLT FGQGTKVEIKR
SEQ ID NO: 421
DIQMTQSPSSLSASVGDRVTITC GASENIYHALN WYQQKPGKAPKLLIY GATNLAH
GVPSRFSGSGSGTDFTLTISSLQPEDFATYYC QNHLHHPLT FGQGTKVEIKR
SEQ ID NO: 422
DIQMTQSPSSLSASVGDRVTITC GASENIYGAHN WYQQKPGKAPKLLIY GATNLAH
GVPSRFSGSGSGTDFTLTISSLQPEDFATYYC QNHLHHPLT FGQGTKVEIKR
SEQ ID NO: 423
DIQMTQSPSSLSASVGDRVTITC GAHENIYHALN WYQQKPGKAPKLLIY GATNLAH
GVPSRFSGSGSGTDFTLTISSLQPEDFATYYC QNHLHHPLT FGQGTKVEIKR
SEQ ID NO: 424
DIQMTQSPSSLSASVGDRVTITC GAHENIYGAHN WYQQKPGKAPKLLIY GATNLAH
GVPSRFSGSGSGTDFTLTISSLQPEDFATYYC QNHLHHPLT FGQGTKVEIKR
SEQ ID NO: 425
DIQMTQSPSSLSASVGDRVTITC GASENIYHAHN WYQQKPGKAPKLLIY GATNLAH
GVPSRFSGSGSGTDFTLTISSLQPEDFATYYC QNHLHHPLT FGQGTKVEIKR
SEQ ID NO: 426
DIQMTQSPSSLSASVGDRVTITC GAHENIYHAHN WYQQKPGKAPKLLIY GATNLAH
GVPSRFSGSGSGTDFTLTISSLQPEDFATYYC QNHLHHPLT FGQGTKVEIKR
SEQ ID NO: 427
ASTKGPSV FPLAPCSRST SESTAALGCL VKDYFPEPVT VSWNSGALTS GVHTFPAVLQ
SSGLYSLSSV VTVPSSNFGT QTYTCNVDHK PSNTKVDKTV ERKCCVECPP CPAPPVAGPS
VFLFPPKPKD TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGK
SEQ ID NO: 428
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 429
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 430
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALKFHYTQKSLSLSPGK
SEQ ID NO: 431
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALKFHYTQKSLSLSPGK
SEQ ID NO: 432
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEVTC
VVVDVSHEDPEVQFNWYVDGVEVHNAKTKPREEQFNSTFRVVSVLTVVHQDWLNGKEYKCKVSNK
GLPAPIEKTISKTKGQPREPQVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPGK
SEQ ID NO: 433
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEVTC
VVVDVSHEDPEVQFNWYVDGVEVHNAKTKPREEQFNSTFRVVSVLTVVHQDWLNGKEYKCKVSNK
GLPAPIEKTISKTKGQPREPQVYTLPPSREEMTKNQVSLTCLVKGFYPSDIAVEWESNGQPENNYKTTPP
MLDSDGSFFLYSKLTVDKSRWQQGNVFSCSVMHEALKFHYTQKSLSLSPGK
SEQ ID NO: 434
TV AAPSVFIFPP SDEQLKSGTA SVVCLLNNFY PREAKVQWKV DNALQSGNSQ ESVTEQDSKD
STYSLSSTLT LSKADYEKHK VYACEVTHQG LSSPVTKSFN RGEC
SEQ ID NO: 435
QVQLVQSGAEVKKPGASVKVSCKASGYIFSNYWIQWVRQAPGQGLEWMGEILPGSGSTEYTENFKDR
VTMTRDTSTSTVYMELSSLRSEDTAVYYCARYHFGSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 436
DIQMTQSPSSLSASVGDRVTITCGAHENIYGALNWYQQKPGKAPKWYGATNLADGVPSRFSGSGSGT
DFTLTISSLQPEDFATYYCQNVLNTPLTFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKS
FNRGEC
SEQ ID NO: 437
QVQLVQSGAEVKKPGASVKVSCKASGHIFSNYWIQWVRQAPGQGLEWMGEILPGSGSTEYTENFKDR
VTMTRDTSTSTVYMELSSLRSEDTAVYYCARYHFGSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 438
QVQLVQSGAEVKKPGASVKVSCKASGYIFSNYWHQWVRQAPGQGLEWMGEILPGSGSTEYTENFKD
RVTMTRDTSTSTVYMELSSLRSEDTAVYYCARYHFGSSPNWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 439
QVQLVQSGAEVKKPGASVKVSCKASGYIFSNYWIQWVRQAPGQGLEWMGEHLPGSGSTEYTENFKD
RVTMTRDTSTSTVYMELSSLRSEDTAVYYCARYHFGSSPNWYFDVWGQGTLVTVSSASTKGPSVFPLA
PSSKSTSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 440
QVQLVQSGAEVKKPGASVKVSCKASGYIFSNYWIQWVRQAPGQGLEWMGEILHGSGSTEYTENFKDR
VTMTRDTSTSTVYMELSSLRSEDTAVYYCARYHFGSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 441
QVQLVQSGAEVKKPGASVKVSCKASGYIFSNYWIQWVRQAPGQGLEWMGEILPGSGHTEYTENFKDR
VTMTRDTSTSTVYMELSSLRSEDTAVYYCARYHFGSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 442
QVQLVQSGAEVKKPGASVKVSCKASGYIFSNYWIQWVRQAPGQGLEWMGEILPGSGSTEYTENFKDR
VTMTRDTSTSTVYMELSSLRSEDTAVYYCARYHFHSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 443
DIQMTQSPSSLSASVGDRVTITCGAHENIYHALNWYQQKPGKAPKLLIYGATNLADGVPSRFSGSGSGT
DFTLTISSLQPEDFATYYCQNVLNTPLTFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKS
FNRGEC
SEQ ID NO: 444
DIQMTQSPSSLSASVGDRVTITC GAHENIYGALN WYQQKPGKAPKLLIY GATNLAD
GVPSRFSGSGSGTDFTLTISSLQPEDFATYYC QNVHNTPLT FGQGTKVEIKR
TVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 445
DIQMTQSPSSLSASVGDRVTITCGASENIYGALNWYQQKPGKAPKLLIYGATNLADGVPSRFSGSGSGT
DFTLTISSLQPEDFATYYCQNVLNTHLTFGQGTKVEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVT
KSFNRGEC
SEQ ID NO: 446
QVQLVQSGAEVKKPGASVKVSCKASGHIFS NYWIQ WVRQAPGQGLEWMG EILPGSGSTEYTENFKD
RVTMTRDTSTSTVYMELSSLRSEDTAVYYCAR YFFGSSPNWYFDV WGQGTLVTVSS
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 447
QVQLVQSGAE VKKPGASVKV SCKASGYIFS NYWIQ WVRQAPGQGLEWMG
EHLPGSGSTEYTENFKD RVTMTRDTSTSTVYMELSSLRSEDTAVYYCAR YFFGSSPNWYFDV
WGQGTLVTVSS
SEQ ID NO: 448
QVQLVQSGAEVKKPGASVKVSCKASGYIFSNYWIQWVRQAPGQGLEWMGEILHGSGSTEYTENFKDR
VTMTRDTSTSTVYMELSSLRSEDTAVYYCARYHFGSSPNWYFDVWGQGTLVTVSS
SEQ ID NO: 449
QVQLVQSGAEVKKPGASVKVSCKASGYIFSNYWIQWVRQAPGQGLEWMGEILHGSGSTEYTENFKDR
VTMTRDTSTSTVYMELSSLRSEDTAVYYCARYFFGSSPNWYFDVWGQGTLVTVSS
SEQ ID NO: 450
QVQLVQSGAEVKKPGASVKVSCKASGYIFSNYWIQWVRQAPGQGLEWMGEILPGSGSTEYTENFKDR
VTMTRDTSTSTVYMELSSLRSEDTAVYYCARYFFHSSPNWYFDVWGQGTLVTVSS
SEQ ID NO: 451
QVQLVQSGAEVKKPGASVKVSCKASGYIFSNYWIQWVRQAPGQGLEWMGEILPGSGSTEYTENFKDR
VTMTRDTSTSTVYMELSSLRSEDTAVYYCARYHFHSSPNWYFDVWGQGTLVTVSS
SEQ ID NO: 452
QVQLVQSGAEVKKPGASVKVSCKASGYTFS HYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALKFHYTQKSLSLSPGK
SEQ ID NO: 453
DIQMTQSPSSLSASVGDRVTITC GAHENIYHALN WYQQKPGKAPKLLIY GATNLAH
GVPSRFSGSGSGTDFTLTISSLQPEDFATYYC QNVLNTPLT FGQGTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
SEQ ID NO: 454
DIQMTQSPSSLSASVGDRVTITC GASENIYGALN WYQQKPGKAPKLLIY GATNLAD
GVPSRFSGSGSGTDFTLTISSLQPEDFATYYC QNVHNTPLT FGQGTKVEIKR
TVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 455
DIQMTQSPSSLSASVGDRVTITC GASENIYGALN WYQQKPGKAPKLLIY GATNLAD
GVPSRFSGSGSGTDFTLTISSLQPEDFATYYC QNVHNTPLT FGQGTKVEIKR
SEQ ID NO: 456
DIQMTQSPSSLSASVGDRVTITC GAHENIYGALN WYQQKPGKAPKLLIY GATNLAD
GVPSRFSGSGSGTDFTLTISSLQPEDFATYYC QNHLNTPLT FGQGTKVEIKR
TVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 457
DIQMTQSPSSLSASVGDRVTITC GAHENIYGALN WYQQKPGKAPKLLIY GATNLAH
GVPSRFSGSGSGTDFTLTISSLQPEDFATYYC QNVLNTPLT FGQGTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
SEQ ID NO: 458
DIQMTQSPSSLSASVGDRVTITC GAHENIYGAHN WYQQKPGKAPKLLIY GATNLAD
GVPSRFSGSGSGTDFTLTISSLQPEDFATYYC QNVHNTPLT FGQGTKVEIKR
TVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 459
DIQMTQSPSSLSASVGDRVTITC GAHENIYGAHN WYQQKPGKAPKLLIY GATNLAD
GVPSRFSGSGSGTDFTLTISSLQPEDFATYYC QNVHNTPLT FGQGTKVEIKR
SEQ ID NO: 460
QVQLVQSGAEVKKPGASVKVSCKASGYTFS HYWIQ WVRQAPGQGLEWMG
EILPGSGSTEYTENFKD RVTMTRDTSISTAYMELSSLRSEDTAVYYCAR YHFGSSPNWYFDV
WGQGTLVTVSS
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
SEQ ID NO: 461
QVQLVQSGAEVKKPGASVKVSCKASGYIFSNYWIQWVRQAPGQGLEWMGEIHPGSGSTEYTENFKDR
VTMTRDTSTSTVYMELSSLRSEDTAVYYCARYHFGSSPNWYFDVWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYIC
NVNHKPSNTKVDKKVEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 462
QVQLVQSGAEVKKPGASVKVSCKASGYIFSNYWIQWVRQAPGQGLEWMGEILPGSGSTEYTENFKDR
VTMTRDTSTSTVYMELSSLRSEDTAVYYCARYHFGSSPNWYFDVWGQGTLVTVSSASTKGPSVFPLAP
CSRSTSESTAALGCLVKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPAPIEKTISKT
KGQPREPQVYTLPPSREEMTKNQVSLTCLVKGFYPSDIAVEWESNGQPENNYKTTPPMLDSDGSFFLYS
KLTVDKSRWQQGNVFSCSVMHEALHNHYTQKSLSLSPGK
SEQ ID NO: 463
DIQMTQSPSSLSASVGDRVTITC GASENIYGALN WYQQKPGKAPKLLIY GATNLAD
GVPSRFSGSGSGTDFTLTISSLQPEDFATYYC QNVLHTPLT FGQGTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC

### Other Embodiments

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1. An antibody, or antigen-binding fragment thereof, comprising (i) a light chain variable region comprising an amino acid sequence at least 90% identical to amino acid residues 1-108 of SEQ ID NO:2 and comprising a histidine at position 26; and (ii) a heavy chain variable region comprising an amino acid sequence at least 90% identical to amino acid residues 1-122 of SEQ ID NO:1 and comprising a histidine at position 100.

2. The antibody, or antigen-binding fragment thereof, of claim 1, wherein the heavy chain variable region further comprises a histidine at one or more of positions 27, 31, 34, 51, 52, 53, 57 and 102 of SEQ ID NO:1.

3. The antibody, or antigen-binding fragment thereof, of claim 2, wherein the heavy chain variable region comprises a histidine at position 34 of SEQ ID NO:1.

4. The antibody, or antigen-binding fragment thereof, of any one of the preceding claims, wherein the light chain variable region further comprises a histidine at one or more of positions 31, 33, 56, 91, 92, and 95 of SEQ ID NO:2.

5. The antibody, or antigen-binding fragment thereof, of claim 4, wherein the light chain variable region comprises a histidine at positions 33 and 93of SEQ ID NO:2.

6. The antibody, or antigen-binding fragment thereof, of claim 4, wherein the light chain variable region comprises a histidine at positions 33 and 92 of SEQ ID NO:2.

7. The antibody, or antigen-binding fragment thereof, of any one of the preceding claims, wherein the heavy chain variable region comprises a threonine at position 28 of SEQ ID NO:1.

8. The antibody, or antigen-binding fragment thereof, of any one of the preceding claims, wherein the heavy chain variable region comprises an isoleucine at position 76 of SEQ ID NO:1.

9. The antibody, or antigen-binding fragment thereof, of any one of the preceding claims, wherein the heavy chain variable region comprises an alanine at position 79 of SEQ ID NO:1.

10. The antibody, or antigen-binding fragment thereof, of any one of the preceding claims, wherein the heavy chain variable region comprises an amino acid sequence selected from SEQ ID NO:222, SEQ ID NO:223, or SEQ ID NO:224.

11. The antibody, or antigen-binding fragment thereof, of any one of the preceding claims, wherein the light chain variable region comprises an amino acid sequence selected from SEQ ID NO:304, SEQ ID NO:311, SEQ ID NO:312, or SEQ ID NO:328.

12. An antibody, or antigen-binding fragment thereof, comprising (i) a heavy chain variable domain comprising amino acid residues 1-122 of SEQ ID NO:1 with a histidine at position 100 and one or more additional amino acid substitutions, and (ii) a light chain variable domain comprising amino acid residues 1-108 of SEQ ID NO:2 with a histidine at position 26 and one or more additional amino acid substitutions.

13. The antibody, or antigen-binding fragment thereof, of claim 12, wherein the one or more additional amino acid substitutions in either the heavy chain variable domain or light chain variable domain is no more than one substitution per chain.

14. The antibody, or antigen-binding fragment thereof, of claim 12, wherein the one or more amino acid substitutions in either the heavy chain variable domain or light chain variable domain is no more than two substitutions per chain.

15. The antibody, or antigen-binding fragment thereof, of claim 12, wherein the one or more amino acid substitutions in either the heavy chain variable domain or light chain variable domain is no more than three substitutions per chain.

16. The antibody, or antigen-binding fragment thereof, of claim 12, wherein the one or more amino acid substitutions in either the heavy chain variable domain or light chain variable domain is no more than four substitutions per chain.

17. The antibody, or antigen-binding fragment thereof, of any one of the preceding claims, further comprising an IgG constant region.

18. The antibody, or antigen-binding fragment thereof claim 17, wherein the IgG constant region is IgG1.

19. The antibody, or antigen-binding fragment thereof, of claim 17, wherein the IgG constant region comprises an alanine at one or both of positions 234 and 235 of a native human IgG constant region, according to EU numbering.

20. The antibody, or antigen-binding fragment thereof, of any one of the preceding claims, further comprising a constant region comprising the amino acid sequence of SEQ ID NO:428.

21. The antibody, or antigen-binding fragment thereof, of claim 20, wherein the constant region comprises an alanine at one or both of positions 117 and 118 of SEQ ID NO:428.

22. An antibody, or antigen-binding fragment thereof, comprising (i) a light chain variable region comprising an amino acid sequence at least 90% identical to SEQ ID NO:304, SEQ ID NO:311, SEQ ID NO:312, or SEQ ID NO:328; and/or (ii) a heavy chain variable region comprising an amino acid sequence at least 90% identical to SEQ ID NO:222, SEQ ID NO:223, or SEQ ID NO:224.

23. The antibody, or antigen-binding fragment thereof, of claim 22, wherein the light chain variable region comprises an amino acid sequence at least 95% identical to SEQ ID NO:304, SEQ ID NO:311, SEQ ID NO:312, or SEQ ID NO:328; and/or (ii) the heavy chain variable region comprises an amino acid sequence at least 95% identical to SEQ ID NO:222, SEQ ID NO:223, or SEQ ID NO:224.

24. The antibody, or antigen-binding fragment thereof, of claim 22, wherein the light chain variable region comprises an amino acid sequence identical to SEQ ID NO:304, SEQ ID NO:311, SEQ ID NO:312, or SEQ ID NO:328; and/or (ii) the heavy chain variable region comprises an amino acid sequence identical to SEQ ID NO:222, SEQ ID NO:223, or SEQ ID NO:224.

25. The antibody, or antigen-binding fragment thereof, of claim 22, wherein the heavy chain variable region comprises a threonine at position 28 of SEQ ID NO:1.

26. The antibody, or antigen-binding fragment thereof, of claim 22-25, wherein the heavy chain variable region comprises an isoleucine at position 76 of SEQ ID NO:1.

27. The antibody, or antigen-binding fragment thereof, of any one of claims 22-26, wherein the heavy chain variable region comprises an alanine at position 79 of SEQ ID NO:1.

28. The antibody, or antigen-binding fragment thereof, of any one of claims 22-27, further comprising an IgG constant region.

29. The antibody, or antigen-binding fragment thereof claim 28, wherein the IgG constant region is IgG1.

30. The antibody, or antigen-binding fragment thereof, of claim 28, wherein the IgG constant region comprises an alanine at one or both of positions 234 and 235 of a native human IgG constant region, according to EU numbering.

31. The antibody, or antigen-binding fragment thereof, of any one of claims 22-27, further comprising a constant region comprising the amino acid sequence of SEQ ID NO:428.

32. The antibody, or antigen-binding fragment thereof, of claim 31, wherein the constant region comprises an alanine at one or both of positions 117 and 118 of SEQ ID NO:428.

33. An antibody, or antigen-binding fragment thereof, comprising (i) a light chain CDR1 comprising the amino acid sequence of SEQ ID NO:279 or SEQ ID NO:279 having one or more amino acid substitutions, (ii) a light chain CDR2 comprising the amino acid sequence of SEQ ID NO:287 or SEQ ID NO:287 having one or more amino acid substitutions, (iii) a light chain CDR3 comprising the amino acid sequence of SEQ ID NO:290 or SEQ ID NO:290 having one or more amino acid substitutions, (iv) a heavy chain CDR1 comprising the amino acid sequence of SEQ ID NO:7 or SEQ ID NO:7 having one or more amino acid substitutions, (v) a heavy chain CDR2 comprising the amino acid sequence of SEQ ID NO:12 or SEQ ID NO:12 having one or more amino acid substitutions, and (vi) a heavy chain CDR3 comprising the amino acid sequence of SEQ ID NO:19 or SEQ ID NO:19 having one or more amino acid substitutions.

34. An antibody, or antigen-binding fragment thereof, comprising (i) a light chain CDR1 comprising the amino acid sequence of SEQ ID NO:279 or SEQ ID NO:279 having one or more amino acid substitutions , (ii) a light chain CDR2 comprising the amino acid sequence of SEQ ID NO:288 or SEQ ID NO:288 having one or more amino acid substitutions, (iii) a light chain CDR3 comprising the amino acid sequence of SEQ ID NO:290 or SEQ ID NO:290 having one or more amino acid substitutions, (iv) a heavy chain CDR1 comprising the amino acid sequence of SEQ ID NO:8 or SEQ ID NO:8 having one or more amino acid substitutions, (v) a heavy chain CDR2 comprising the amino acid sequence of SEQ ID NO:12 or SEQ ID NO:12 having one or more amino acid substitutions, and (vi) a heavy chain CDR3 comprising the amino acid sequence of SEQ ID NO:19 or SEQ ID NO:19 having one or more amino acid substitutions.

35. The antibody, or antigen-binding fragment thereof, of claim 33 or 34, wherein the one or more amino acid substitutions in each individual CDR is no more than one substitution.

36. The antibody, or antigen-binding fragment thereof, of claim 33 or 34, wherein the one or more amino acid substitutions in each individual CDR is no more than two substitutions.

37. The antibody, or antigen-binding fragment thereof, of claim 33 or 34, wherein the one or more amino acid substitutions in each individual CDR is no more than three substitutions.

38. The antibody, or antigen-binding fragment thereof, of any one of claims 33-37, wherein (i) the light chain CDR1 comprises the amino acid sequence of SEQ ID NO:279 having a histidine at one or more of positions 8, 10, 11, 31, 33, or 34, (ii) the light chain CDR2 comprises the amino acid sequence of SEQ ID NO:287 having a histidine at one ore more of positions 4, 5, or 7, or (iii) the light chain CDR3 comprises the amino acid sequence of SEQ ID NO:290 having a histidine at one or more of positions 3, 5, or 6.

39. The antibody, or antigen-binding fragment thereof, of any one of claims 33-39, wherein (i) the heavy chain CDR1 comprises the amino acid sequence of SEQ ID NO:7 having a histidine at position 1, 3, or 4, (ii) the heavy chain CDR2 comprises the amino acid sequence of SEQ ID NO:12 having a histidine at position 2, 3 or 8, or (iii) the heavy chain CDR3 comprises the amino acid sequence of SEQ ID NO:18 having a histidine at positions 2, 4, or 7.

40. An antibody, or antigen-binding fragment thereof, comprising (i) a light chain CDR1 defined by amino acid sequence of SEQ ID NO:279, SEQ ID NO: 282, SEQ ID NO:283, or 285 (ii) a light chain CDR2 defined by amino acid sequence of SEQ ID NO:287 or SEQ ID NO:288, (iii) a light chain CDR3 defined by amino acid sequence of SEQ ID NO:290, SEQ ID NO:291, or SEQ ID NO:294, (iv) a heavy chain CDR1 defined by amino acid sequence of SEQ ID NO:7, SEQ ID NO:8. or SEQ ID NO:10, (v) a heavy chain CDR2 defined by amino acid sequence of SEQ ID NO:12, SEQ ID NO:13, or SEQ ID NO:15, and (vi) a heavy chain CDR3 defined by amino acid sequence of SEQ ID NO:18, or SEQ ID NO:19.

41. The antibody, or antigen-binding fragment thereof, of any one of claims 33-40, further comprising an IgG constant region.

42. The antibody, or antigen-binding fragment thereof claim 41, wherein the IgG constant region is IgG1.

43. The antibody, or antigen-binding fragment thereof, of claim 441, wherein the IgG constant region comprises an alanine at one or both of positions 234 and 235 of a native human IgG constant region, according to EU numbering.

44. The antibody, or antigen-binding fragment thereof, of any one of claims 33-40, further comprising a constant region comprising the amino acid sequence of SEQ ID NO:428.

45. The antibody, or antigen-binding fragment thereof, of claim 44, wherein the constant region comprises an alanine at one or both of positions 117 and 118 of SEQ ID NO:428.

46. The antibody, or antigen-binding fragment thereof, of any one of the preceding claims, wherein the antibody or antigen-binding fragment thereof binds to complement component human C5.

47. The antibody, or antigen-binding fragment thereof, of any one of the preceding claims, wherein the antibody or antigen-binding fragment thereof inhibits cleavage of C5 into fragments C5a and C5b.

48. The antibody, or antigen-binding fragment thereof, of any one of the preceding claims, wherein the antibody or antigen-binding fragment thereof binds to human C5 at pH 7.4 with an affinity dissociation constant (K_{D}) of 0.1 nM to 1 nM.

49. The antibody, or antigen-binding fragment thereof, of any one of the preceding claims, wherein the antibody, or antigen-binding fragment thereof, binds to human C5 at pH 7.4 with an affinity dissociation constant (K_{D}) of less than 0.45 nM, less than 0.40 nM, less than 0.35 nM, less than 0.30 nM, or less than 0.25 nM.

50. The antibody, or antigen-binding fragment thereof, of any one of the preceding claims, wherein the antibody or antigen-binding fragment thereof binds to human C5 at pH 5.5 with a K_{D} of 25 to 200 nM.

51. The antibody, or antigen-binding fragment thereof, of any one of the preceding claims, wherein the antibody, or antigen-binding fragment thereof, binds to human C5 at pH 5.5 with a K_{D} of greater than 25 nM, greater than 50 nM, greater than 75 nM, greater than 100 nM, greater than 125 nM, greater than 150 nM, or greater than 175 nM.

52. The antibody, or antigen-binding fragment thereof, of any one of the preceding claims, wherein the [(K_{D} of the antibody or antigen-binding fragment thereof for human C5 at pH 5.5)/(K_{D} of the antibody or antigen-binding fragment thereof for human C5 at pH 7.4)] is 50 to 750.

53. The antibody, or antigen-binding fragment thereof, of any one of the preceding claims, wherein the [(K_{D} of the antibody or antigen-binding fragment thereof for human C5 at pH 5.5)/(K_{D} of the antibody or antigen-binding fragment thereof for human C5 at pH 7.4)] is greater than 50, greater than 100, greater than 150, greater than 200, greater than 250, greater than 300, greater than 350, greater than 400, greater than 450, greater than 500, greater than 550, grater than 600, or greater than 700.

54. The antibody, or antigen-binding fragment thereof, of any one of the preceding claims, wherein the off rate of human C5 from the antibody or antigen-binding fragment thereof at pH 5.5 is greater than 0.05 s⁻¹.

55. The antibody, or antigen-binding fragment thereof, of any one of the preceding claims, wherein the off rate of human C5 from the antibody or antigen-binding fragment thereof at pH 5.5 is greater than 0.1 s⁻¹, greater than 0.15 s⁻¹, greater than 0.2 s⁻¹, greater than 0.25 s⁻¹, greater than 0.3 s⁻¹, greater than 0.35 s⁻¹, or greater than 0.4 s⁻¹.

56. The antibody, or antigen-binding fragment thereof, of any one of the preceding claims, wherein the antibody, or antigen-binding fragment thereof has a serum half-life of about 5 days, about 10 days, about 15 days, about 20 days, about 25 days, about 30 days, about 35 days, about 40 days, about 45 days, about 50 days or longer.

57. A nucleic acid sequence encoding an antibody or antigen-binding fragment thereof of any one of claims 1-56.

58. A vector comprising the nucleic acid sequence of claim 57.

59. A host cell comprising the nucleic acid sequence of claim 57 or the vector of claim 58.

60. A method of producing an antibody, or antigen-binding fragment thereof, comprising culturing the host cell of claim 59 under conditions suitable for expression of the antibody or antigen-binding fragment thereof.

61. An antibody, or antigen binding fragment thereof according to claims 1-56, for use as a medicament.

62. A method of treating a complement-mediated disease or disorder, the method comprising administering to a subject in need thereof an effective amount of the antibody, or antigen-binding fragment thereof, of any one of claims 1-56.
